(19) United States
(12) Patent Application Publication
(10) Pub. No.: US 2020/0094068 A1
(43) Pub. Date: Mar. 26, 2020

(54) METHOD FOR TREATMENT OF NON-ALCOHOLIC STEATOHEPATITIS USING PULSED ELECTROMAGNETIC FIELD THERAPY

(71) Applicant: Endonovo Therapeutics, Inc., Woodland Hills, CA (US)

(72) Inventors: Andre' A. DIMINO, Woodcliff Lake, NJ (US); Arthur A. PILLA, Oakland, NJ (US); Iyer VISWANATHAN, Santa Clara, CA (US)

(21) Appl. No.: 16/657,827

(22) Filed: Oct. 18, 2019

Related U.S. Application Data
(63) Continuation-in-part of application No. 15/607,211, filed on May 26, 2017.
Application No. 15/607,211 is a continuation of application No. 13/801,789, filed on Mar. 13, 2013, now abandoned.
Application No. 13/801,789 is a continuation of application No. 12/819,956, filed on Jun. 21, 2010, now abandoned.
Application No. 12/819,956 is a continuation-in-part of application No. 12/772,002, filed on Apr. 30, 2010, now abandoned.
Application No. 12/772,002 is a continuation of application No. 11/003,108, filed on Dec. 3, 2004, now Pat. No. 7,744,524.
Application No. 12/819,956 is a continuation-in-part of application No. 11/114,666, filed on Apr. 26, 2005, now Pat. No. 7,740,574.
Application No. 12/819,956 is a continuation-in-part of application No. 11/110,000, filed on Apr. 19, 2005, now abandoned.
Application No. 12/819,956 is a continuation-in-part of application No. 11/369,308, filed on Mar. 6, 2006, now abandoned.
Application No. 12/819,956 is a continuation-in-part of application No. 11/369,309, filed on Mar. 6, 2006, now abandoned.
Application No. 12/819,956 is a continuation-in-part of application No. 11/223,073, filed on Sep. 10, 2005, now Pat. No. 7,758,490.
Application No. 11/223,073 is a continuation-in-part of application No. 11/339,204, filed on Jan. 25, 2006, now abandoned.
Application No. 11/339,204 is a continuation-in-part of application No. 11/818,065, filed on Jun. 12, 2007, now abandoned.
Application No. 12/819,956 is a continuation-in-part of application No. 11/903,294, filed on Sep. 20, 2007, now abandoned.
Application No. 12/819,956 is a continuation-in-part of application No. 11/977,043, filed on Oct. 22, 2007, now abandoned.
Application No. 11/977,043 is a continuation-in-part of application No. 16/431,561, filed on Jun. 4, 2019.
Application No. 16/431,561 is a continuation of application No. 14/932,928, filed on Nov. 4, 2015, now Pat. No. 10,350,428.
(60) Provisional application No. 60/527,327, filed on Dec. 5, 2003.
(60) Provisional application No. 60/564,887, filed on Apr. 26, 2004.
(60) Provisional application No. 60/563,104, filed on Apr. 19, 2004.
(60) Provisional application No. 60/658,967, filed on Mar. 7, 2005.
(60) Provisional application No. 60/658,968, filed on Mar. 7, 2005.
(60) Provisional application No. 60/812,841, filed on Jun. 12, 2006.
(60) Provisional application No. 60/846,126, filed on Sep. 20, 2006.
(60) Provisional application No. 60/852,927, filed on Oct. 20, 2006.
(60) Provisional application No. 62/747,529, filed on Oct. 18, 2018.
(60) Provisional application No. 62/075,122, filed on Nov. 4, 2014.

(51) Int. Cl. A61N 2/02; A61N 1/40

(52) U.S. Cl. CPC A61N 2/02; A61N 2/008; A61N 1/40

(57) ABSTRACT

A method for controlling levels of glucose and lipids in the blood of a patient having non-alcoholic steatohepatitis (NASH) includes generating an electromagnetic signal and coupling the electromagnetic signal to a target structure in the patient's liver to a induce a pulsed magnetic field in the target structure. The signal may include bursts of sinusoidal, rectangular, chaotic, and/or random waveforms, having a frequency content in a range of about 0.01 Hz to about 100 MHz at about 1 to about 100,000 waveforms per second, a burst duration from about 1 μsec to about 100 msec, a burst repetition rate from about 0.01 to about 1000 bursts/second. The induced magnetic field may have a peak amplitude of between 2 and 20 μT. The treatment may be repeated between 1 and 3 times per day every day for at least one week.

### CROSS-REFERENCE TO RELATED APPLICATIONS

This application claims the benefit under 35 U.S.C. § 119 of U.S. Provisional Patent Application No. 62/747529, filed Oct. 18, 2018, entitled “METHOD FOR TREATMENT OF NON-ALCOHOLIC STEATOHEPATITIS USING PULSED ELECTROMAGNETIC FIELD THERAPY.”

In addition, this application is a continuation-in-part of U.S. patent application Ser. No. 15/607,211, entitled “APPARATUS AND METHOD FOR ELECTROMAGNETIC TREATMENT”, Publication No. US 2018-0104505 A1, which is a continuation of U.S. patent application Ser. No. 13/801,789, filed Mar. 13, 2016, entitled “APPARATUS AND METHOD FOR ELECTROMAGNETIC TREATMENT”, Publication No. US 2013-0274540 A1, which is a continuation of U.S. patent application Ser. No. 12/819,956, filed Jun. 21, 2010, entitled “APPARATUS AND METHOD FOR ELECTROMAGNETIC TREATMENT,” Publication No. US-2011-0112352-A1, now abandoned, which is a continuation-in-part of U.S. patent application Ser. No. 12/772,002, filed Apr. 30, 2010, entitled “APPARATUS AND METHOD FOR ELECTROMAGNETIC TREATMENT OF PLANT, ANIMAL AND HUMAN TISSUE, ORGANS, CELLS AND MOLECULES,” Publication No. US-2010-0222631-A1, which is a continuation of U.S. patent application Ser. No. 11/003,108, filed Dec. 3, 2004, entitled “APPARATUS AND METHOD FOR ELECTROMAGNETIC TREATMENT OF PLANT, ANIMAL, AND HUMAN TISSUE, ORGANS, CELLS, AND MOLECULES,” now U.S. Pat. No. 7,744,524, which claims the benefit under 35 U.S.C. § 119 of U.S. Provisional Patent Application No. 60/527,327, filed Dec. 5, 2003, entitled “APPARATUS AND METHOD FOR ELECTROMAGNETIC TREATMENT OF PLANT, ANIMAL, AND HUMAN TISSUE, ORGANS, CELLS AND MOLECULES.”

U.S. patent application Ser. No. 12/819,956 is also a continuation-in-part of U.S. patent application Ser. No. 11/114,666, filed Apr. 26, 2005, entitled “ELECTROMAGNETIC TREATMENT INDUCTION APPARATUS AND METHOD FOR USING SAME,” now U.S. Pat. No. 7,740,574, which claims the benefit under 35 U.S.C. § 119 of U.S. Provisional Patent Application No. 60/564,887, filed Apr. 26, 2004, entitled “INDUCTION MEANS FOR THERAPEUTICALLY TREATING HUMAN AND ANIMAL CELLS, TISSUES AND ORGANS WITH ELECTROMAGNETIC FIELDS.”

U.S. patent application Ser. No. 12/819,956 is also a continuation-in-part of U.S. patent application Ser. No. 11/110,000, filed Apr. 19, 2005, entitled “ELECTROMAGNETIC TREATMENT APPARATUS AND METHOD FOR ANGIOGENESIS MODULATION OF LIVING TISSUES AND CELLS,” now abandoned, which claims the benefit under 35 U.S.C. § 119 of U.S. Provisional Patent Application No. 60/563,104, filed Apr. 19, 2004, entitled “APPARATUS AND METHOD FOR THERAPEUTICALLY TREATING HUMAN AND ANIMAL CELLS, TISSUES AND ORGANS WITH ELECTROMAGNETIC FIELDS.”

U.S. patent application Ser. No. 12/819,956 is also a continuation-in-part of U.S. patent application Ser. No. 11/369,308, filed Mar. 6, 2006, entitled “ELECTROMAGNETIC TREATMENT APPARATUS FOR AUGMENTING WOUND REPAIR AND METHOD FOR USING SAME,” Publication No. US-2006-0212077-A1, now abandoned, which claims the benefit under 35 U.S.C. § 119 of U.S. Provisional Patent Application No. 60/658,967, filed Mar. 7, 2005, entitled “APPARATUS AND METHOD FOR THERAPEUTICALLY TREATING HUMAN, ANIMAL, AND PLANT CELLS, TISSUES, ORGANS, AND MOLECULES WITH ELECTROMAGNETIC FIELDS FOR WOUND REPAIR.”

U.S. patent application Ser. No. 12/819,956 is also a continuation-in-part of U.S. patent application Ser. No. 11/369,309, filed Mar. 6, 2006, entitled “ELECTROMAGNETIC TREATMENT APPARATUS FOR ENHANCING PHARMACOLOGICAL, CHEMICAL AND TOPICAL AGENT EFFECTIVENESS AND METHOD FOR USING SAME,” Publication No. US-2007-0026514-A1, now abandoned, which claims the benefit under 35 U.S.C. § 119 of U.S. Provisional Patent Application No. 60/658,968, filed Mar. 7, 2005, entitled “APPARATUS AND METHOD FOR TREATING HUMAN, ANIMAL AND PLANT CELLS, TISSUES, ORGANS AND MOLECULES WITH ELECTROMAGNETIC FIELDS BY ENHANCING THE EFFECTS OF PHARMACOLOGICAL, CHEMICAL, COSMETIC AND TOPICAL AGENTS.”

U.S. patent application Ser. No. 12/819,956 is also a continuation-in-part of U.S. patent application Ser. No. 11/223,073, filed Sep. 10, 2005, entitled “INTEGRATED COIL APPARATUS FOR THERAPEUTICALLY TREATING HUMAN AND ANIMAL CELLS, TISSUES AND ORGANS WITH ELECTROMAGNETIC FIELDS AND METHOD FOR USING SAME,” now U.S. Pat. No. 7,758,490.

U.S. patent application Ser. No. 12/819,956 is also a continuation-in-part of U.S. patent application Ser. No. 11/339,204, filed Jan. 25, 2006, entitled “SELF-CONTAINED ELECTROMAGNETIC APPARATUS FOR TREATMENT OF MOLECULES, CELLS, TISSUES, AND ORGANS WITHIN A CEREBROFACIAL AREA AND METHOD FOR USING SAME,” Publication No. US-2007-0173904-A1, now abandoned.

U.S. patent application Ser. No. 12/819,956 is also a continuation-in-part of U.S. patent application Ser. No. 11/818,065, filed Jun. 12, 2007, entitled “ELECTROMAGNETIC APPARATUS FOR PROPHYLAXIS AND REPAIR OF OPHTHALMIC TISSUE AND METHOD FOR USING SAME,” Publication No. US-2008-0058793-A1, now abandoned, which claims the benefit under 35 U.S.C. § 119 of U.S. Provisional Patent Application No. 60/812,841, filed Jun. 12, 2006, entitled “APPARATUS AND METHOD FOR THERAPEUTICALLY TREATING HUMAN AND ANIMAL CELLS, TISSUES, ORGANS AND MOLECULES WITH ELECTROMAGNETIC FIELDS FOR TREATMENT OF DISEASES OF THE EYE AND PROPHYLACTIC TREATMENT OF THE EYE.”

U.S. patent application Ser. No. 12/819,956 is also a continuation-in-part of U.S. patent application Ser. No. 11/903,294, filed Sep. 20, 2007, entitled “ELECTROMAGNETIC APPARATUS FOR RESPIRATORY DISEASE AND METHOD FOR USING SAME,” Publication No. US-2008-0132971-A1, now abandoned, which claims the benefit under 35 U.S.C. § 119 of “APPARATUS AND METHOD FOR THE TREATMENT OF DISEASES OF THE LUNGS WITH ELECTROMAGNETIC FIELDS.”

U.S. patent application Ser. No. 12/819,956 is also a continuation-in-part of U.S. patent application Ser. No. 11/977,043, filed Oct. 22, 2007, entitled “APPARATUS AND METHOD FOR THE TREATMENT OF EXCESSIVE FIBROUS CAPSULE FORMATION AND CAPSULAR CONTRACTURE WITH ELECTROMAGNETIC FIELDS,” Publication No. US-2008-0140155-A1, which claims the benefit under 35 U.S.C. § 119 of U.S. Provisional Patent Application No. 60/852,927, filed Oct. 20, 2006, entitled “APPARATUS AND METHOD FOR THE TREATMENT OF EXCESSIVE FIBROUS CAPSULE FORMATION AND CAPSULAR CONTRACTURE WITH ELECTROMAGNETIC FIELDS.”

This application is also a continuation-in-part of U.S. patent application Ser. No. 16/431,561, filed Jun. 4, 2019, entitled “METHOD AND APPARATUS FOR ELECTROMAGNETIC TREATMENT OF LIVING SYSTEMS”, which is a continuation of U.S. patent application Ser. No. 14/932,928, filed Nov. 4, 2015, entitled “METHOD AND APPARATUS FOR ELECTROMAGNETIC TREATMENT OF LIVING SYSTEMS”, now U.S. Pat. No. 10,350,428, which claims the benefit under 35 U.S.C. § 119 of U.S. Provisional Patent Application No. 62/075,122, filed Nov. 4, 2014, entitled “METHOD AND APPARATUS FOR ELECTROMAGNETIC TREATMENT OF LIVING SYSTEMS.”

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

### Field of the Invention

The disclosure relates generally to the field of therapeutic devices and methods for treating patients, and more specifically to a method for treatment of non-alcoholic steatohepatitis using pulsed electromagnetic field therapy.

### Background Art

1. Pulsed Electromagnetic Field Therapy

It is now well established that application of weak non-thermal electromagnetic fields (“EMF”) can result in physiologically meaningful in vivo and in vitro bioeffects. Time-varying electromagnetic fields, comprising rectangular waveforms such as pulsing electromagnetic fields (“PEMF”), and sinusoidal waveforms such as pulsed radio frequency fields (“PRF”) ranging from several Hertz to an about 15 to an about 40 MHz range, are clinically beneficial when used as an adjunctive therapy for a variety of musculoskeletal injuries and conditions.

Beginning in the 1960's, development of modern therapeutic and prophylactic devices was stimulated by clinical problems associated with non-union and delayed union bone fractures. Early work showed that an electrical pathway can be a means through which bone adaptively responds to mechanical input. Early therapeutic devices used implanted and semi-invasive electrodes delivering direct current (“DC”) to a fracture site. Non-invasive technologies were subsequently developed using electrical and electromagnetic fields. These modalities were originally created to provide a non-invasive “no-touch” means of inducing an electrical/mechanical waveform at a cell/tissue level. Clinical applications of these technologies in orthopaedics have led to approved applications by regulatory bodies worldwide for treatment of fractures such as non-unions and fresh fractures, as well as spine fusion. Presently several EMF devices constitute the standard armamentarium of orthopaedic clinical practice for treatment of difficult to heal fractures. The success rate for these devices has been very high. The database for this indication is large enough to enable its recommended use as a safe, non-surgical, non-invasive alternative to a first bone graft. Additional clinical indications for these technologies have been reported in double blind studies for treatment of avascular necrosis, tendinitis, osteoarthritis, wound repair, blood circulation and pain from arthritis as well as other musculoskeletal injuries.

Cellular studies have addressed effects of weak low frequency electromagnetic fields on both signal transduction pathways and growth factor synthesis. It can be shown that EMF stimulates secretion of growth factors after a short, trigger-like duration. Ion/ligand binding processes at a cell membrane are generally considered an initial EMF target pathway structure. The clinical relevance to treatments for example of bone repair, is upregulation such as modulation, of growth factor production as part of normal molecular regulation of bone repair. Cellular level studies have shown effects on calcium ion transport, cell proliferation, Insulin Growth Factor (“IGF-II”) release, and IGF-II receptor expression in osteoblasts. Effects on Insulin Growth Factor-I (“IGF-I”) and IGF-II have also been demonstrated in rat fracture callus. Stimulation of transforming growth factor beta (“TGFβ”) messenger RNA (“mRNA”) with PEMF in a bone induction model in a rat has been shown. Studies have also demonstrated upregulation of TGF-β mRNA by PEMF in human osteoblast-like cell line designated MG-63, wherein there were increases in TGF-β1, collagen, and osteocalcin synthesis. PEMF stimulated an increase in TGF-β1 in both hypertrophic and atrophic cells from human non-union tissue. Further studies demonstrated an increase in both TGF-β1 mRNA and protein in osteoblast cultures resulting from a direct effect of EMF on a calcium/calmodulin-dependent pathway. Cartilage cell studies have shown similar increases in TGF-β1 mRNA and protein synthesis from EMF, demonstrating a therapeutic application to joint repair. U.S. Pat. No. 4,315,503 (1982) to Ryaby and U.S. Pat. No. 5,723,001 (1998) to Pilla typify the research conducted in this field.

However, prior art in this field applies unnecessarily high amplitude and power to a target pathway structure, requires unnecessarily long treatment time, and is not portable.

Therefore, a need exists for an apparatus and a method that more effectively modulates biochemical processes that regulate tissue growth and repair, shortens treatment times, and incorporates miniaturized circuitry and light weight applicators thus allowing the apparatus to be portable and if desired disposable. A further need exists for an apparatus and method that more effectively modulates biochemical processes that regulate tissue growth and repair, shortens treatment times, and incorporates miniaturized circuitry and light weight applicators that can be constructed to be implantable.

EMF has been used in applications of bone repair and bone healing. Waveforms comprising low frequency components and low power are currently used in orthopedic clinics. Origins of using bone repair signals began by considering that an electrical pathway may constitute a means through which bone can adaptively respond to EMF signals. A linear physicochemical approach employing an electrochemical model of a cell membrane predicted a range of EMF waveform patterns for which bioeffects might be expected. Since a cell membrane was a likely EMF target, it became necessary to find a range of waveform parameters for which an induced electric field could couple electrochemically at the cellular surface, such as voltage-dependent kinetics. Extension of this linear model also involved Lorentz force analysis.

A pulsed radio frequency (“PRF”) signal derived from a 27.12 MHz continuous sine wave used for deep tissue healing is known in the prior art of diathermy. A pulsed successor of the diathermy signal was originally reported as an electromagnetic field capable of eliciting a non-thermal biological effect in the treatment of infections. PRF therapeutic applications have been reported for reduction of post-traumatic and post-operative pain and edema in soft tissues, wound healing, burn treatment and nerve regeneration. Application of EMF for the resolution of traumatic edema has become increasingly used in recent years. Results to date using PRF in animal and clinical studies suggest that edema may be measurably reduced from such electromagnetic stimulus.

Prior art considerations of EMF dosimetry have not taken into account dielectric properties of tissue structure as opposed to the properties of isolated cells.

In recent years, clinical use of non-invasive PRF at radio frequencies comprised using pulsed bursts of a 27.12 MHz sinusoidal wave, wherein each pulse burst comprises a width of sixty-five microseconds, having approximately 1,700 sinusoidal cycles per burst, and various burst repetition rates. This limited frequency components that could couple to relevant dielectric pathways in cells and tissue.

Time-varying electromagnetic fields, comprising rectangular waveforms such as pulsing electromagnetic fields, and sinusoidal waveforms such as pulsed radio frequency fields ranging from several Hertz to an about 15 to an about 40 MHz range, are clinically beneficial when used as an adjunctive therapy for a variety of musculoskeletal injuries and conditions.

Beginning in the 1960's, development of modern therapeutic and prophylactic devices was stimulated by clinical problems associated with non-union and delayed union bone fractures. Early work showed that an electrical pathway can be a means through which bone adaptively responds to mechanical input. Early therapeutic devices used implanted and semi-invasive electrodes delivering direct current (“DC”) to a fracture site. Non-invasive technologies were subsequently developed using electrical and electromagnetic fields. These modalities were originally created to provide a non-invasive “no-touch” means of inducing an electrical/mechanical waveform at a cell/tissue level. Clinical applications of these technologies in orthopaedics have led to approved applications by regulatory bodies worldwide for treatment of fractures such as non-unions and fresh fractures, as well as spine fusion. Presently several EMF devices constitute the standard armamentarium of orthopaedic clinical practice for treatment of difficult to heal fractures. The success rate for these devices has been very high. The database for this indication is large enough to enable its recommended use as a safe, non-surgical, non-invasive alternative to a first bone graft. Additional clinical indications for these technologies have been reported in double blind studies for treatment of avascular necrosis, tendinitis, osteoarthritis, wound repair, blood circulation and pain from arthritis as well as other musculoskeletal injuries.

Cellular studies have addressed effects of weak low frequency electromagnetic fields on both signal transduction pathways and growth factor synthesis. It can be shown that EMF stimulates secretion of growth factors after a short, trigger-like duration. Ion/ligand binding processes at a cell membrane are generally considered an initial EMF target pathway structure. The clinical relevance to treatments for example of bone repair, is upregulation such as modulation, of growth factor production as part of normal molecular regulation of bone repair. Cellular level studies have shown effects on calcium ion transport, cell proliferation, Insulin Growth Factor (“IGF-II”) release, and IGF-II receptor expression in osteoblasts. Effects on Insulin Growth Factor-I (“IGF-I”) and IGF-II have also been demonstrated in rat fracture callus. Stimulation of transforming growth factor beta (“TGF-β”) messenger RNA (“mRNA”) with PEMF in a bone induction model in a rat has been shown. Studies have also demonstrated upregulation of TGF-β mRNA by PEMF in human osteoblast-like cell line designated MG-63, wherein there were increases in TGF-62 1, collagen, and osteocalcin synthesis. PEMF stimulated an increase in TGF-62 1 in both hypertrophic and atrophic cells from human non-union tissue. Further studies demonstrated an increase in both TGF-62 1 mRNA and protein in osteoblast cultures resulting from a direct effect of EMF on a calcium/calmodulin-dependent pathway. Cartilage cell studies have shown similar increases in TGF-62 1 mRNA and protein synthesis from EMF, demonstrating a therapeutic application to joint repair. Various studies conclude that upregulation of growth factor production may be a common denominator in the tissue level mechanisms underlying electromagnetic stimulation. When using specific inhibitors, EMF can act through a calmodulin-dependent pathway. It has been previously reported that specific PEMF and PRF signals, as well as weak static magnetic fields, modulate Ca.sup.2+ binding to CaM in a cell-free enzyme preparation. Additionally, upregulation of mRNA for BMP2 and BMP4 with PEMF in osteoblast cultures and upregulation of TGF-62 1 in bone and cartilage with PEMF have been demonstrated.

However, prior art in this field does not use an induction apparatus that is lightweight, portable, disposable, implantable, and configured with, integrated into, or attached to at least one of garments, fashion accessories, footwear, bandages, anatomical supports, an anatomical wraps, apparel, cushions, mattresses, pads, wheelchairs, therapeutic beds, therapeutic chairs, therapeutic and health maintenance devices such as vacuum assisted wound closure devices, mechanical and functional electrical stimulation devices and exercise devices, ultrasound, heat, cold, massage, and exercise.

Therefore, a need exists for an electromagnetic treatment induction apparatus and a method for using same that is lightweight, portable, implantable, and can be disposable. A further need exists for an electromagnetic treatment induction apparatus and method that can be used more effectively with miniaturized circuitry that optimally configures electromagnetic waveforms to be inductively coupled with plant, animal, and human tissue, organs, cells, and molecules for therapeutic treatment.

As mentioned above, by use of a substantially single voltage amplitude envelope with each PRF burst, one was limiting frequency components that could couple to relevant dielectric pathways in cells and tissue.

However, prior art in this field does not configure waveforms based upon a ion/ligand binding transduction pathway. Prior art waveforms are inefficient since prior art waveforms apply unnecessarily high amplitude and power to living tissues and cells, require unnecessarily long treatment time, and cannot be generated by a portable device.

Therefore, a need exists for an apparatus and a method that more effectively modulates angiogenesis and other biochemical processes that regulate tissue growth and repair, shortens treatment times, and incorporates miniaturized circuitry and light weight applicators thus allowing the apparatus to be portable and if desired disposable. A further need exists for an apparatus and method that more effectively modulates angiogenesis and other biochemical processes that regulate tissue growth and repair, shortens treatment times, and incorporates miniaturized circuitry and light weight applicators that can be constructed to be implantable.

Time-varying electromagnetic fields, comprising either rectangular, pseudo-rectangular, or both rectangular and pseudo-rectangular waveforms, such as pulse modulated electromagnetic fields, and sinusoidal waveforms such as pulsed radio frequency fields ranging from several Hertz to an about 15 to an about 40 MHz range, are clinically beneficial when used as an adjunctive therapy for a variety of musculoskeletal injuries and conditions.

However, prior art in this field does not use an induction apparatus that delivers a signal according to a mathematical model, is programmable, lightweight, portable, disposable, implantable, and configured with, integrated into, or attached to at least one of garments, fashion accessories, footwear, bandages, anatomical supports, an anatomical wraps, apparel, cushions, mattresses, pads, wheelchairs, therapeutic beds, therapeutic chairs, therapeutic and health maintenance devices such as vacuum assisted wound closure devices, mechanical and functional electrical stimulation devices and exercise devices, ultrasound, heat, cold, massage, and exercise. A further need exists for an electromagnetic treatment induction apparatus and a method for using same that is lightweight, portable, implantable, and can be disposable. A further need exists for an electromagnetic treatment induction apparatus and method having decreased power requirements and non-invasive characteristics that allow an enhanced signal to be integrated into surgical dressings, wound dressings, pads, seat cushions, mattress pads, shoes, and any other garment and structure juxtaposed to living tissue and cells, even to be integral to creation of a garment to deliver an enhanced EMF signal to any body parts and that delivers a signal according to a mathematical model and is programmable.

Prior art equipment in this field is bulky, not designed for outdoor use, and not self-contained.

In some embodiments, the proposed EMF transduction pathway relevant to tissue maintenance, repair and regeneration, begins with voltage-dependent Ca2+ binding to CaM, which is favored when cytosolic Ca2+ homeostasis is disrupted by chemical and/or physical insults at the cellular level. Ca/CaM binding produces activated CaM that binds to, and activates, cNOS, which catalyzes the synthesis of the signaling molecule NO from L-arginine. This pathway is shown in its simplest schematic form in FIG. 1A. FIG. 1A is a schematic summary of the body's primary anti-inflammatory cascade and the proposed manner by which PEMF may accelerate postoperative pain relief. Surgical injury increases cytosolic Ca2+, which activates CaM. PEMF accelerates CaM activation thereby enhancing NO/cGMP anti-inflammatory signaling. PEMF also enhances CaM-dependent PDE activation, which accelerates cGMP inhibition. PEMF dosing must take into account the competing dynamics of NO/cGMP signaling and PDE inhibition of cGMP.

As shown in FIG. 36A, cNOS* represents activated constitutive nitric oxide synthase (cNOS), which catalyzes the production of NO from L-arginine, which, in turn, activates soluble gyanylyl cyclase, sGC. The term “sGC*” refers to activated guanylyl cyclase which catalyzes cyclic guanosine monophosphate (cGMP) formation when NO signaling modulates the tissue repair pathway. “AC*” refers to activated adenylyl cyclase, which catalyzes cyclic adenosine monophosphate (cAMP) when NO signaling modulates differentiation and survival.

According to some embodiments, an EMF signal can be configured to accelerate cytosolic ion binding to a cytosolic buffer, such as voltage dependent Ca2+ binding to CaM, because the rate constant for binding, kₒₙ is much greater than the rate constant for unbinding, koff, imparting rectifier-like properties to ion-buffer binding, such as Ca2+ binding to CaM.

Yet another embodiment pertains to application of sinusoidal, rectangular, chaotic or arbitrary waveform electromagnetic signals, having frequency components below about 100 GHz, configured to accelerate the binding of intracellular Ca2+ to a buffer, such as CaM, to enhance biochemical signaling pathways in animal and human cells and tissues. Signals configured according to additional embodiments produce a net increase in a bound ion, such as Ca2+, at CaM binding sites because the asymmetrical kinetics of Ca/CaM binding allows such signals to accumulate voltage induced at the ion binding site, thereby accelerating voltage-dependent ion binding. Examples of therapeutic and prophylactic applications are modulation of biochemical signaling in anti-inflammatory pathways, modulation of biochemical signaling in cytokine release pathways, modulation of biochemical signaling in growth factor release pathways; edema and lymph reduction, anti-inflammatory, post-surgical and post-operative pain and edema relief, nerve, bone and organ pain relief, increased local blood flow, microvascular blood perfusion, treatment of tissue and organ ischemia, brain tissue ischemia from stroke or traumatic brain injury, treatment of neurological injury and neurodegenerative diseases such as Alzheimer's and Parkinson's; angiogenesis, neovascularization; enhanced immune response; enhanced effectiveness of pharmacological agents; nerve regeneration; prevention of apoptosis; modulation of heat shock proteins for prophylaxis and response to injury or pathology.

In some variations the systems, devices and/or methods generally relate to application of electromagnetic fields (EMF), and in particular, pulsed electromagnetic fields (PEMF), including a subset of PEMF in a radio frequency domain (e.g., pulsed radio frequency or PRF), for the treatment of any of the applications disclosed herein in animals and humans, including pain, edema, tissue repair and head, cerebral and neural injury, and neurodegenerative conditions.

Transient elevations in cytosolic Ca2+, from external stimuli as simple as changes in temperature and receptor activation, or as complex as mechanical disruption of tissue, will activate CaM. Once Ca2+ ions are bound, a conformational change will allow CaM bind to and activate a number of key enzymes involved in cell viability and function, such as the endothelial and neuronal constitutive nitric oxide synthases (cNOS); eNOS and nNOS, respectively. As a consequence, NO is rapidly produced, albeit in lower concentrations than the explosive increases in NO produced by inducible NOS (iNOS), during the inflammatory response. In contrast, these smaller, transient increases in NO produced by Ca/CaM-binding will activate soluble guanylyl cyclase (sGC), which will catalyze the formation of cyclic guanosine monophosphate (cGMP). The CaM/NO/cGMP signaling pathway can rapidly modulate blood flow in response to normal physiologic demands, as well as to inflammation. Importantly, this same pathway will also rapidly attenuate expression of cytokines such as interleukin-1 beta (IL-1β), and iNOS and stimulate anti-apoptotic pathways in neurons. All of these effects are mediated by calcium and cyclic nucleotides, which in turn regulate growth factors such as basic fibroblast growth factor (FGF-2) and vascular endothelial growth factor (VEGF), resulting in pleiotrophic effects on cells involved in tissue repair and maintenance. PEMF can also accelerate the inhibition of cGMP by phosphodiesterase (PDE) Improved PEMF signal configurations and treatment regimens are disclosed herein that can minimize the inhibition of cGMP by PDE.

Therefore, a need exists for an apparatus and a method that modulates the biochemical pathways that regulate animal and human tissue response to maximize the rate of cGMP production while minimizing the rate of inhibition of cGMP. In some embodiments, an apparatus incorporates miniaturized circuitry and light weight coil applicators or electrodes to deliver any of the waveforms described herein thus allowing the apparatus to be low cost, portable and, if desired, disposable.
2. Non-Alcoholic Steatohepatitis

Steatohepatitis is a type of fatty liver disease, characterized by inflammation of the liver, concurrent with fat accumulation in the liver. There are two primary types of steatophepatitis: alcoholic steathohepatitis, caused by excessive alcohol intake, and nonalcoholic steathohepatitis (NASH), the exact cause of which is not known, but is linked to a variety of factors including obesity, insulin resistance, type 2 diabetes, high cholesterol, high triglycerides, and metabolic syndrome. There is currently no standard treatment for NASH, but doctors frequently recommend managing conditions by making lifestyle changes such as losing weight, increasing exercise, controlling diabetes, and reducing cholesterol intake.

### SUMMARY OF THE INVENTION

Various apparatus, methods, devices, and systems are described herein. The summary is set forth in twelve parts (parts 1-12). Each part may be considered internally consistent, however, embodiments, ranges, features, elements, and illustrations from one part may be used in combination (in whole or in part) with embodiments, ranges, features, elements, and illustrations from another part or parts. Although there is some repetition in the language in each of these parts, this disclosure is intended to illustrate different variations and embodiments of the devices, systems, and methods for electrically stimulating tissue to treat various disorders, as described in greater detail herein.

### Part 1

Described herein are apparatus and methods for delivering electromagnetic signals to human, animal and plant target pathway structures such as molecules, cells, tissue and organs for therapeutic and prophylactic purposes. A preferred embodiment according to the present disclosure utilizes a Power Signal to Noise Ratio (“Power SNR”) approach to configure bioeffective waveforms and incorporates miniaturized circuitry and lightweight flexible coils. This advantageously allows a device that utilizes a Power SNR approach, miniaturized circuitry, and lightweight flexible coils, to be completely portable and if desired to be constructed as disposable and if further desired to be constructed as implantable.

Specifically, broad spectral density bursts of electromagnetic waveforms, configured to achieve maximum signal power within a bandpass of a biological target, are selectively applied to target pathway structures such as living organs, tissues, cells and molecules. Waveforms are selected using a unique amplitude/power comparison with that of thermal noise in a target pathway structure. Signals comprise bursts of at least one of sinusoidal, rectangular, chaotic and random wave shapes, have frequency content in a range of about 0.01 Hz to about 100 MHz at about 1 to about 100,000 bursts per second, and have a burst repetition rate from about 0.01 to about 1000 bursts/second. Peak signal amplitude at a target pathway structure such as tissue, lies in a range of about 1 μV/cm to about 100 mV/cm. Each signal burst envelope may be a random function providing a means to accommodate different electromagnetic characteristics of healing tissue. A preferred embodiment according to the present comprises a 20 millisecond pulse burst comprising about 5 to about 20 microsecond symmetrical or asymmetrical pulses repeating at about 1 to about 100 kilohertz within the burst. The burst envelope is a modified 1/f function and is applied at random repetition rates. A resulting waveform can be delivered via inductive or capacitive coupling.

It is an object of the present disclosure to configure a power spectrum of a waveform by mathematical simulation by using signal to noise ratio (“SNR”) analysis to configure an optimized, bioeffective waveform then coupling the configured waveform using a generating device such as ultra-lightweight wire coils that are powered by a waveform configuration device such as miniaturized electronic circuitry.

It is another object of the present disclosure to evaluate Power SNR for any target pathway structure such as molecules, cells, tissues and organs of plants, animals and humans using any input waveform, even if the electrical equivalents are non-linear as in a Hodgkin-Huxley membrane model.

It is another object of the present disclosure to provide a method and apparatus for treating plants, animals and humans using electromagnetic fields selected by optimizing a power spectrum of a waveform to be applied to a chosen biochemical target pathway structure such as a molecule, cell, tissue and organ of a plant, animal, and human.

It is another object of the present disclosure to employ significantly lower peak amplitudes and shorter pulse duration. This can be accomplished by matching via Power SNR, a frequency range in a signal to frequency response and sensitivity of a target pathway structure such as a molecule, cell, tissue, and organ, of plants, animals and humans.

### Part 2

An electromagnetic treatment induction apparatus and a method for using same for therapeutic treatment of living tissues and cells by inductively coupling optimally configured waveforms to alter the living tissues and cells' interaction with their electromagnetic environment.

According to an embodiment of the present disclosure, by treating a selectable body region with a flux path comprising a succession of EMF pulses having a minimum width characteristic of at least about 0.01 microseconds in a pulse burst envelope having between about 1 and about 100,000 pulses per burst, in which a voltage amplitude envelope of said pulse burst is defined by a randomly varying parameter in which instantaneous minimum amplitude thereof is not smaller than the maximum amplitude thereof by a factor of one ten-thousandth. The pulse burst repetition rate can vary from about 0.01 to about 10,000 Hz. A mathematically definable parameter can also be employed to define an amplitude envelope of said pulse bursts.

By increasing a range of frequency components transmitted to relevant cellular pathways, access to a large range of biophysical phenomena applicable to known healing mechanisms, including enhanced enzyme activity and growth factor and cytokine release, is advantageously achieved.

According to an embodiment of the present disclosure, by applying a random, or other high spectral density envelope, to a pulse burst envelope of mono- or bi-polar rectangular or sinusoidal pulses which induce peak electric fields between 10⁻⁶ and 10 volts per centimeter (V/cm), a more efficient and greater effect can be achieved on biological healing processes applicable to both soft and hard tissues in humans, animals and plants. A pulse burst envelope of higher spectral density can advantageously and efficiently couple to physiologically relevant dielectric pathways, such as, cellular membrane receptors, ion binding to cellular enzymes, and general transmembrane potential changes thereby modulating angiogenesis and neovascularization.

By advantageously applying a high spectral density voltage envelope as a modulating or pulse-burst defining parameter, power requirements for such modulated pulse bursts can be significantly lower than that of an unmodulated pulse. This is due to more efficient matching of the frequency components to the relevant cellular/molecular process. Accordingly, the dual advantages of enhanced transmitting dosimetry to relevant dielectric pathways and of decreasing power requirements are achieved.

A preferred embodiment according to the present disclosure comprises about 0.1 to about 100 millisecond pulse burst comprising about 1 to about 200 microsecond symmetrical or asymmetrical pulses repeating at about 0.1 to about 100 kilohertz within the burst. The burst envelope is a modified 1/f function and is applied at random repetition rates between about 0.1 and about 1000 Hz. Fixed repetition rates can also be used between about 0.1 Hz and about 1000 Hz. An induced electric field from about 0.001 mV/cm to about 100 mV/cm is generated. Another embodiment according to the present disclosure comprises an about 0.01 millisecond to an about 10 millisecond burst of high frequency sinusoidal waves, such as 27.12 MHz, repeating at about 1 to about 100 bursts per second. An induced electric field from about 0.001 mV/cm to about 100 mV/cm is generated. Resulting waveforms can be delivered via inductive or capacitive coupling.

It is another object of the present disclosure to provide an electromagnetic method of treatment of living cells and tissues comprising a broad-band, high spectral density electromagnetic field.

It is a further object of the present disclosure to provide an electromagnetic method of treatment of living cells and tissues comprising amplitude modulation of a pulse burst envelope of an electromagnetic signal that will induce coupling with a maximum number of relevant EMF-sensitive pathways in cells or tissues.

It is an object of the present disclosure to configure a power spectrum of a waveform by mathematical simulation by using signal to noise ratio (“SNR”) analysis to configure a waveform optimized to modulate angiogenesis and neovascularization then coupling the configured waveform using a generating device such as ultra-lightweight wire coils that are powered by a waveform configuration device such as miniaturized electronic circuitry.

It is an object of the present disclosure to provide lightweight flexible coils, that can be placed in at least one of garments, fashion accessories, footwear, bandages, anatomical supports, an anatomical wraps, apparel, cushions, mattresses, pads, wheelchairs, therapeutic beds, therapeutic chairs, therapeutic and health maintenance devices such as vacuum assisted wound closure devices, mechanical and functional electrical stimulation devices and exercise devices and dressings to deliver the optimum dose of non-invasive pulsed electromagnetic treatment configured as shown above, for enhanced repair and growth of living tissue in animals, humans and plants.

It is another object of the present disclosure to provide multiple coils, delivering a waveform configured by SNR/Power analysis of a target pathway, to increase area of treatment coverage.

It is another object of the present disclosure to provide multiple coils that are simultaneously driven or that are sequentially driven such as multiplexed, with the same or different optimally configured waveforms as shown above.

It is a further object of the present disclosure to provide flexible, lightweight coils that focus the EMF signal to the affected tissue by incorporating the coils, delivering a waveform configured by SNR/Power analysis of a target pathway, into ergonomic support garments.

It is yet a further object of the present disclosure to utilize conductive thread to create daily wear, and exercise and sports garments having integrated coils, delivering a waveform configured by SNR/Power analysis of a target pathway, positioned in proximity to an anatomical target.

It is yet a further object of the present disclosure to utilize lightweight flexible coils or conductive thread to deliver the EMF signal to affected tissue by incorporating such coils or conductive threads as an integral part of various types of bandages, such as, compression, elastic, cold compress and hot compress and delivering a waveform configured by SNR/Power analysis of a target pathway.

It is another object of the present disclosure to employ several coils, delivering a waveform configured by SNR/Power analysis of a target pathway, to increase EMF coverage area.

It is another object of the present disclosure to construct a coil, delivering a waveform configured by SNR/Power analysis of a target pathway, using conductive thread.

It is another object of the present disclosure to construct a coil, delivering a waveform configured by SNR/Power analysis of a target pathway, using fine flexible conductive wire.

It is another object of the present disclosure to supply the same or different waveforms configured by SNR/Power analysis of a target pathway, simultaneously or sequentially to single or multiple coils.

It is yet a further object of the present disclosure to incorporate at least one coil in a surgical wound dressing to apply an enhanced EMF signal non-invasively and non-surgically, the surgical wound dressing to be used in combination with standard wound treatment.

It is another object of the present disclosure to construct the coils delivering a waveform configured by SNR/Power analysis of a target pathway, for easy attachment and detachment to dressings, garments and supports by using an attachment means such as Velcro, an adhesive and any other such temporary attachment means.

It is another object of the present disclosure to provide coils delivering a waveform configured by SNR/Power analysis of a target pathway, that are integrated with therapeutic beds, therapeutic chairs, and wheelchairs.

It is another object of the present disclosure to provide coils delivering a waveform configured by SNR/Power analysis of a target pathway, that are integrated with various therapy surfaces, such as pressure relieving, inflatable, fluid, visco-elastic and air fluidized bed and other support surfaces.

It is another object of the present disclosure to provide coils delivering a waveform configured by SNR/Power analysis of a target pathway that are integrated with therapeutic seat cushions such as inflatable, fluidized, foam cushions.

It is another object of the present disclosure to provide coils delivering a waveform configured by SNR/Power analysis of a target pathway, that are integrated with at least one of therapeutic mattress overlays, sheets, blankets, pillows, pillow cases, and therapeutic devices that can apply steady or intermittent pressure such as air clearance vests.

It is another object of the present disclosure to provide for the inclusion of a flux path to any therapeutic surface, structure, or device to enhance the effectiveness of such therapeutic surfaces, structures or devices by delivering a waveform configured by SNR/Power analysis of a target pathway.

It is another object of the present disclosure to incorporate coils delivering a waveform configured by SNR/Power analysis of a target pathway, in footwear such as shoes.

It is another object of the present disclosure to integrate at least one coil delivering a waveform configured by SNR/Power analysis of a target pathway, with a therapeutic surface, structure or device to enhance the effectiveness of such therapeutic surface, structure or device.

### Part 3

Also described herein are apparatus and methods for electromagnetic treatment of living tissues and cells by altering their interaction with their electromagnetic environment.

It is an object of the present disclosure to provide modulation of electromagnetically sensitive regulatory processes at the cell membrane and at junctional interfaces between cells.

It is another object of the present disclosure to provide increased blood flow to affected tissues by modulating angiogenesis and neovascularization.

It is another object of the present disclosure to provide increased blood flow to enhance viability, growth, and differentiation of implanted cells, such as stem cells, tissues and organs.

It is another object of the present disclosure to provide increased blood flow in cardiovascular diseases by modulating angiogenesis and neovascularization.

It is another object of the present disclosure to improve micro-vascular blood perfusion and reduced transudation.

It is another object of the present disclosure to provide a treatment of maladies of the bone and other hard tissue by modulating angiogenesis and neovascularization.

It is a still further object of the present disclosure to provide a treatment of edema and swelling of soft tissue by increased blood flow through modulation of angiogenesis and neovascularization.

It is another object of the present disclosure to provide an electromagnetic method of treatment of living cells and tissues that can be used for repair of damaged soft tissue.

It is yet another object of the present disclosure to increase blood flow to damaged tissue by modulation of vasodilation and stimulating neovascularization.

It is a yet further object of the present disclosure to provide an apparatus for modulation of angiogenesis and neovascularization that can be operated at reduced power levels and still possess benefits of safety, economics, portability, and reduced electromagnetic interference.

It is another object of the present disclosure to modulate angiogenesis and neovascularization by evaluating Power SNR for any target pathway structure such as molecules, cells, tissues and organs of plants, animals and humans using any input waveform, even if electrical equivalents are non-linear as in a Hodgkin-Huxley membrane model.

It is another object of the present disclosure to provide a method and apparatus for treating plants, animals and humans using electromagnetic fields selected by optimizing a power spectrum of a waveform to be applied to a biochemical target pathway structure to enable modulation of angiogenesis and neovascularization within molecules, cells, tissues and organs of a plant, animal, and human.

It is another object of the present disclosure to significantly lower peak amplitudes and shorter pulse duration. This can be accomplished by matching via Power SNR, a frequency range in a signal to frequency response and sensitivity of a target pathway structure such as a molecule, cell, tissue, and organ, of plants, animals and humans to enable modulation of angiogenesis and neovascularization.

### Part 4

The present disclosure relates to accelerating wound repair of living tissues, cells and molecules by providing a therapeutic, prophylactic and wellness apparatus and method for non-invasive pulsed electromagnetic treatment to enhance condition, repair and growth of living tissue in animals, humans and plants. This beneficial method operates to selectively change a bio-electromagnetic environment associated with cellular and tissue environments by using electromagnetic means such as EMF generators and applicator heads. An embodiment according to the present disclosure comprises introducing a flux path to a selectable body region, comprising a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter in which an instantaneous minimum amplitude thereof is not smaller than a maximum amplitude thereof by a factor of one ten thousandth. Further, the repetition rate of such pulse bursts may vary from 0.01 to 10,000 Hertz. A mathematically definable parameter satisfying SNR and/or Power SNR detectability requirements in a target structure is employed to define the configuration of the pulse bursts.

It is another object of the present disclosure to provide a method of treating living cells and tissue by electromagnetically modulating sensitive regulatory processes at a cell membrane and at junctional interfaces between cells, using waveforms configured to satisfy SNR and Power SNR detectability requirements in a target pathway structure.

Specifically, broad spectral density bursts of electromagnetic waveforms, configured to achieve maximum signal power within a bandpass of a biological target, are selectively applied to target pathway structures such as tissues, to enhance effectiveness of pharmacological, chemical, cosmetic and topical agents. Waveforms are selected using a unique amplitude/power comparison with that of thermal noise in a target pathway structure. Signals comprise bursts of at least one of sinusoidal, rectangular, chaotic and random wave shapes, have frequency content in a range of about 0.01 Hz to about 100 MHz at about 1 to about 100,000 bursts per second, and have a burst repetition rate from about 0.01 to about 1000 bursts/second. Peak signal amplitude at a target pathway structure such as organs, cells, tissues, and molecules, lies in a range of about 1 μV/cm to about 100 mV/cm. Each signal burst envelope may be a random function providing a means to accommodate different electromagnetic characteristics of enhancing bioeffective processes. A preferred embodiment according to the present disclosure comprises about 0.1 to about 100 millisecond pulse burst comprising about 1 to about 200 microsecond symmetrical or asymmetrical pulses repeating at about 0.1 to about 100 kilohertz within the burst. The burst envelope is a modified 1/f function and is applied at random repetition rates between about 0.1 and about 1000 Hz. Fixed repetition rates can also be used between about 0.1 Hz and about 1000 Hz. An induced electric field from about 0.001 mV/cm to about 100 mV/cm is generated. Another embodiment according to the present disclosure comprises an about 0.01 millisecond to an about 10 millisecond burst of high frequency sinusoidal waves, such as 27.12 MHz, repeating at about 1 to about 100 bursts per second. An induced electric field from about 0.001 mV/cm to about 100 mV/cm is generated. Resulting waveforms can be delivered via inductive or capacitive coupling.

It is another object of the present disclosure to provide electromagnetic treatment for wound repair having a broad-band, high spectral density electromagnetic field configured according to at least one of SNR and Power SNR.

It is another object of the present disclosure to accelerate wound repair by configuring a power spectrum of a waveform by mathematical simulation by using signal to noise ratio (“SNR”) analysis to configure a waveform optimized to modulate angiogenesis and neovascularization, then coupling the configured waveform using a generating device such as ultra-lightweight wire coils that are powered by a waveform configuration device such as miniaturized electronic circuitry.

It is another object of the present disclosure to modulate angiogenesis and neovascularization by evaluating Power SNR at any target pathway structure such as molecules, cells, tissues and organs to accelerate wound repair by using any input waveform, even if electrical equivalents are non-linear as in a Hodgkin-Huxley membrane model.

It is another object of the present disclosure to provide an apparatus that incorporates use of Power SNR in which amplitude modulation of the pulse burst envelope of the electromagnetic signal will induce coupling with a maximum number of relevant EMF-sensitive pathways in cells and tissues to enhance wound repair in humans, animals and plants.

It is another object of the present disclosure to provide a method and apparatus for enhancing wound repair using electromagnetic fields selected by optimizing a power spectrum of a waveform to be applied to a biochemical target pathway structure to enable modulation of angiogenesis and neovascularization within molecules, cells, tissues and organs.

It is another object of the present disclosure to significantly lower peak amplitudes and shorter pulse duration by matching via Power SNR, a frequency range in a signal to frequency response and sensitivity of a target pathway structure such as a molecule, cell, tissue, and organ thereby enabling modulation of angiogenesis and neovascularization for accelerating wound repair.

It is another object of the disclosure to provide a method of enhancing soft tissue and hard tissue repair.

It is another object of the disclosure to provide a method of increasing blood flow to affected tissues by modulating angiogenesis.

It is another object of the disclosure to provide an improved method of increasing blood flow to enhance the viability and growth or differentiation of implanted cells, tissues and organs.

It is another object of the disclosure to provide an improved method of increasing blood flow in cardiovascular diseases by modulating angiogenesis.

It is another object of the disclosure to provide beneficial physiological effects through improvement of micro-vascular blood perfusion and reduced transudation.

It is another object of the disclosure to provide an improved method of treatment of maladies of the bone and other hard tissue.

It is a still further object of the disclosure to provide an improved means of the treatment of edema and swelling of soft tissue.

It is another object to provide a means of repair of damaged soft tissue.

It is yet another object to provide a means of increasing blood flow to damaged tissue by modulation of vasodilation and stimulating neovascularization.

It is yet another object to enhance healing of post-surgical wounds by reducing the inflammatory phase and modulating growth factor release.

It is yet another object of the instant disclosure to reduce the inflammatory phase post-cosmetic surgery.

It is yet another object of the instant disclosure to reduce or eliminate the post-surgical complications of breast augmentation, such as capsular contractions.

It is yet another object of the instant disclosure to reduce post-surgical pain, edema and discoloration.

It is yet a further object of the present disclosure to treat chronic wounds such as diabetic ulcers, venous stasis ulcers, pressure sores and any non-healing wound with EMF signals configured according to an embodiment of the present disclosure.

It is a yet a further object to provide apparatus for use of an electromagnetic method of the character indicated, wherein operation of the apparatus can proceed at reduced power levels as compared to those of related methods known in electromedicine and respective biofield technologies, with attendant benefits of safety, economics, portability, and reduced electromagnetic interference.

It is a further object of the present disclosure to provide a method for treatment to enhance wellness.

It is a further object of the present disclosure to provide a method in which electromagnetic waveforms are configured according to SNR and Power SNR detectability requirements in a target pathway structure.

It is another object of the present disclosure to provide a method for electromagnetic treatment comprising a broadband, high spectral density electromagnetic field.

It is another object of the present disclosure to provide a method of enhancing soft tissue and hard tissue repair by using EMF.

It is another object of the present disclosure to provide a method to increase blood flow to affected tissues by using electromagnetic treatment to modulate angiogenesis.

It is yet a further object of the present disclosure to provide a method of treatment of chronic wounds such as diabetic ulcers, venous stasis ulcers, pressure sores and any non-healing wound.

It is another object of the present disclosure to provide a method to increase blood flow to regulate viability, growth, and differentiation of implanted cells, tissues and organs.

It is another object of the present disclosure to provide a method to treat cardiovascular diseases by modulating angiogenesis and increasing blood flow.

It is another object of the present disclosure to provide a method to improve micro-vascular blood perfusion and reduce transudation.

It is another object of the present disclosure to provide a method to increase blood flow to treat maladies of bone and hard tissue.

It is another object of the present disclosure to provide a method to increase blood flow to treat edema and swelling of soft tissue.

It is another object of the present disclosure to provide a method to increase blood flow to repair damaged soft tissue.

It is another object of the present disclosure to provide a method to increase blood flow to damaged tissue by modulation of vasodilation and stimulating neovascularization.

It is a further object of the present disclosure to provide an electromagnetic treatment apparatus wherein the apparatus operates using reduced power levels.

It is a yet further object of the present disclosure to provide an electromagnetic treatment apparatus wherein the apparatus is inexpensive, portable, and produces reduced electromagnetic interference.

### Part 5

The present disclosure relates to enhancing effectiveness of pharmacological, chemical, cosmetic and topical agents used to treat living tissues, cells and molecules by providing a therapeutic, prophylactic and wellness apparatus and method for non-invasive pulsed electromagnetic treatment to enhance condition, repair and growth of living tissue in animals, humans and plants. This beneficial method operates to selectively change a bio-electromagnetic environment associated with cellular and tissue environments by using electromagnetic means such as EMF generators and applicator heads. An embodiment according to the present disclosure comprises introducing a flux path to a selectable body region, comprising a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter in which an instantaneous minimum amplitude thereof is not smaller than a maximum amplitude thereof by a factor of one ten thousandth. Further, the repetition rate of such pulse bursts may vary from 0.01 to 10,000 Hertz. A mathematically definable parameter satisfying SNR and/or Power SNR detectability requirements in a target structure is employed to define the configuration of the pulse bursts. Mathematically defined parameters are selected by considering the dielectric properties of the target pathway structure, and the ratio of the induced electric field amplitude with respect to voltage due to thermal noise or other baseline cellular activity.

It is another object of the present disclosure to provide a method of treating living cells and tissue by electromagnetically modulating sensitive regulatory processes at a cell membrane and at junctional interfaces between cells, using waveforms configured to satisfy SNR and Power SNR detectability requirements in a target pathway structure.

It is another object of the present disclosure to enhance effectiveness of pharmacological, chemical, cosmetic and topical agents by configuring a power spectrum of a waveform by mathematical simulation by using signal to noise ratio (“SNR”) analysis to configure a waveform optimized to modulate angiogenesis and neovascularization, then coupling the configured waveform using a generating device such as ultra-lightweight wire coils that are powered by a waveform configuration device such as miniaturized electronic circuitry.

It is another object of the present disclosure to modulate angiogenesis and neovascularization by evaluating Power SNR at any target pathway structure such as molecules, cells, tissues and organs to enhance effectiveness of pharmacological, chemical, cosmetic and topical agents, by using any input waveform, even if electrical equivalents are non-linear as in a Hodgkin-Huxley membrane model.

It is another object of the present disclosure to provide an apparatus that incorporates use of Power SNR to regulate and adjust electromagnetic therapy treatment to enhance effectiveness of pharmacological, chemical, cosmetic and topical agents.

It is another object of the present disclosure to provide a method and apparatus for enhancing effectiveness of pharmacological, chemical, cosmetic and topical agents using electromagnetic fields selected by optimizing a power spectrum of a waveform to be applied to a biochemical target pathway structure to enable modulation of angiogenesis and neovascularization within molecules, cells, tissues and organs.

It is another object of the present disclosure to significantly lower peak amplitudes and shorter pulse duration by matching via Power SNR, a frequency range in a signal to frequency response and sensitivity of a target pathway structure such as a molecule, cell, tissue, and organ thereby enabling modulation of angiogenesis and neovascularization for enhancing effectiveness of pharmacological, chemical, cosmetic and topical agents.

It is a further object of the present disclosure to provide an apparatus for application of electromagnetic waveforms, to be used in conjunction with pharmacological, chemical, cosmetic and topical agents applied to, upon or in human, animal and plant cells, organs, tissues and molecules so that bioeffective processes of such compounds can be enhanced.

It is a further object of the present disclosure to provide a method to enhance effectiveness of pharmacological, chemical, cosmetic and topical agents for therapeutic, prophylactic and wellness ends.

It is a further object of the present disclosure to provide a method for treatment of organs, muscles, joints, skin and hair using EMF in conjunction with pharmacological, chemical, cosmetic and topical agents to improve the agents' effectiveness.

It is a further object of the present disclosure to provide a method for treatment of organs, muscles, joints, skin and hair using EMF in conjunction with pharmacological, chemical, cosmetic and topical agents to enhance wellness.

It is a further object of the present disclosure to provide a method in which electromagnetic waveforms are configured according to SNR and Power SNR detectability requirements in a target pathway structure.

It is another object of the present disclosure to provide a method for electromagnetic treatment comprising a broadband, high spectral density electromagnetic field.

It is another object of the present disclosure to provide a method of enhancing soft tissue and hard tissue repair by using EMF in conjunction with pharmacological, chemical, cosmetic and topical agents.

It is another object of the present disclosure to provide a method to enhance effectiveness of pharmacological, chemical, cosmetic and topical agents by increasing blood flow to affected tissues by using electromagnetic treatment to modulate angiogenesis.

It is another object of the present disclosure to provide a method to increase blood flow for enhancing effectiveness of pharmacological, chemical, cosmetic and topical agents that regulate viability, growth, and differentiation of implanted cells, tissues and organs.

It is another object of the present disclosure to provide a method to treat cardiovascular diseases by modulating angiogenesis and increasing blood flow to enhance effectiveness of pharmacological, chemical, cosmetic and topical agents.

It is another object of the present disclosure to provide a method that increases physiological effectiveness of pharmacological, chemical, cosmetic and topical agents by improving micro-vascular blood perfusion and reduced transudation.

It is another object of the present disclosure to provide a method to increase blood flow to enhance effectiveness of pharmacological, chemical, cosmetic and topical agents used for treating maladies of bone and hard tissue.

It is another object of the present disclosure to provide a method to increase blood flow to enhance effectiveness of pharmacological, chemical, cosmetic and topical agents used for treating edema and swelling of soft tissue.

It is another object of the present disclosure to provide a method to increase blood flow to enhance effectiveness of pharmacological, chemical, cosmetic and topical agents used for repairing damaged soft tissue.

It is another object of the present disclosure to provide a method to increase blood flow to damaged tissue by modulation of vasodilation and stimulating neovascularization whereby enhanced effectiveness of pharmacological, chemical, cosmetic and topical agents is achieved.

### Part 6

An electromagnetic treatment induction apparatus integrated into therapeutic and non-therapeutic devices and a method for using same for therapeutic treatment of living tissues and cells by inductively coupling optimally configured waveforms to alter the living tissues and cells' interaction with their electromagnetic environment.

The lightweight flexible coils can be an integral portion of a positioning device such as surgical dressings, wound dressings, pads, seat cushions, mattress pads, shoes, wheelchairs, chairs, and any other garment and structure juxtaposed to living tissue and cells. By advantageously integrating a coil into a positioning device therapeutic treatment can be provided to living tissue and cells in an inconspicuous and convenient manner

Specifically, broad spectral density bursts of electromagnetic waveforms, configured to achieve maximum signal power within a bandpass of a biological target, are selectively applied to target pathway structures such as living organs, tissues, cells and molecules. Waveforms are selected using a unique amplitude/power comparison with that of thermal noise in a target pathway structure. Signals comprise bursts of at least one of sinusoidal, rectangular, chaotic and random wave shapes, have frequency content in a range of about 0.01 Hz to about 100 MHz at about 1 to about 100,000 bursts per second, and have a burst repetition rate from about 0.01 to about 1000 bursts/second. Peak signal amplitude at a target pathway structure such as tissue, lies in a range of about 1 μV/cm to about 100 mV/cm. Each signal burst envelope may be a random function providing a means to accommodate different electromagnetic characteristics of healing tissue. A preferred embodiment according to the present disclosure comprises about 0.1 to about 100 millisecond pulse burst comprising about 1 to about 200 microsecond symmetrical or asymmetrical pulses repeating at about 0.1 to about 100 kilohertz within the burst. The burst envelope is a modified 1/f function and is applied at random repetition rates between about 0.1 and about 1000 Hz. Fixed repetition rates can also be used between about 0.1 Hz and about 1000 Hz. An induced electric field from about 0.001 mV/cm to about 100 mV/cm is generated. Another embodiment according to the present disclosure comprises an about 0.01 millisecond to an about 10 millisecond burst of high frequency sinusoidal waves, such as 27.12 MHz, repeating at about 1 to about 100 bursts per second. An induced electric field from about 0.001 mV/cm to about 100 mV/cm is generated. Resulting waveforms can be delivered via inductive or capacitive coupling.

It is another object of the present disclosure to deliver a waveform configured by SNR/Power analysis of a target pathway structure, in a programmable manner for example according to a time-dose program, a series of pulses, or some other sequence random or patterned.

It is another object of the present disclosure to generate a signal from a waveform configured by SNR/Power analysis of a target pathway structure, in a programmable manner for example according to a time-dose program, a series of pulses, or some other sequence random or patterned.

It is yet another object of the present disclosure to integrate at least one coil delivering a waveform configured by Power SNR analysis of a target pathway structure, with at least one of a therapeutic surface, a therapeutic structure, and a therapeutic device, to enhance the effectiveness of the at least one of the therapeutic surface, the therapeutic structure, and the therapeutic device, to prevent the loss and deterioration of cells and tissues.

It is yet another object of the present disclosure to integrate at least one coil delivering a waveform configured by Power SNR analysis of a target pathway structure, with at least one of a therapeutic surface, a therapeutic structure, and a therapeutic device, to enhance the effectiveness of the at least one of the therapeutic surface, the therapeutic structure, and the therapeutic device, to augment cell and tissue activity.

It is yet another object of the present disclosure to integrate at least one coil delivering a waveform configured by Power SNR analysis of a target pathway structure, with at least one of a therapeutic surface, a therapeutic structure, and a therapeutic device, to enhance the effectiveness of the at least one of the therapeutic surface, the therapeutic structure, and the therapeutic device, to increase cell population.

It is yet another object of the present disclosure to integrate at least one coil delivering a waveform configured by Power SNR analysis of a target pathway structure, with at least one of a therapeutic surface, a therapeutic structure, and a therapeutic device, to enhance the effectiveness of the at least one of the therapeutic surface, the therapeutic structure, and the therapeutic device, to prevent neuron deterioration.

It is yet another object of the present disclosure to integrate at least one coil delivering a waveform configured by Power SNR analysis of a target pathway structure, with at least one of a therapeutic surface, a therapeutic structure, and a therapeutic device, to enhance the effectiveness of the at least one of the therapeutic surface, the therapeutic structure, and the therapeutic device, to increase neuron population.

It is yet another object of the present disclosure to integrate at least one coil delivering a waveform configured by Power SNR analysis of a target pathway structure, with at least one of a therapeutic surface, a therapeutic structure, and a therapeutic device, to enhance the effectiveness of the at least one of the therapeutic surface, the therapeutic structure, and the therapeutic device, to prevent deterioration of adrenergic neurons in a cerebrofacial area.

It is yet another object of the present disclosure to integrate at least one coil delivering a waveform configured by Power SNR analysis of a target pathway structure, with at least one of a therapeutic surface, a therapeutic structure, and a therapeutic device, to enhance the effectiveness of the at least one of the therapeutic surface, the therapeutic structure, and the therapeutic device, to increase adrenergic neuron population in a cerebrofacial area.

### Part 7

An apparatus and a method for electromagnetic treatment of hair and other cerebrofacial molecules, cells, organs, tissue, ions, and ligands by altering their interaction with their electromagnetic environment.

By increasing a range of frequency components transmitted to relevant cellular pathways, hair and other cerebrofacial tissue restoration is advantageously achieved.

According to an embodiment of the present disclosure, by applying a random, or other high spectral density envelope, to a pulse burst envelope of mono- or bi-polar rectangular or sinusoidal pulses which induce peak electric fields between 10-8 and 10 volts per centimeter (V/cm), a more efficient and greater effect can be achieved on biological healing processes applicable to both soft and hard tissues in humans, animals and plants. A pulse burst envelope of higher spectral density can advantageously and efficiently couple to physiologically relevant dielectric pathways, such as, cellular membrane receptors, ion binding to cellular enzymes, and general transmembrane potential changes thereby growing, restoring and maintaining hair and other cerebrofacial tissue.

Specifically, broad spectral density bursts of electromagnetic waveforms, configured to achieve maximum signal power within a bandpass of a biological target, are selectively applied to target pathway structures such as hair and other cerebrofacial tissues. Waveforms are selected using a unique amplitude/power comparison with that of thermal noise in a target pathway structure. Signals comprise bursts of at least one of sinusoidal, rectangular, chaotic and random wave shapes, have frequency content in a range of about 0.01 Hz to about 100 MHz at about 1 to about 100,000 bursts per second, and have a burst repetition rate from about 0.01 to about 1000 bursts/second. Peak signal amplitude at a target pathway structure such as hair and or cerebrofacial tissue, lies in a range of about 1 μV/cm to about 100 mV/cm. Each signal burst envelope may be a random function providing a means to accommodate different electromagnetic characteristics of healing tissue. A preferred embodiment according to the present disclosure comprises about 0.1 to about 100 millisecond pulse burst comprising about 1 to about 200 microsecond symmetrical or asymmetrical pulses repeating at about 0.1 to about 100 kilohertz within the burst. The burst envelope is a modified 1/f function and is applied at random repetition rates between about 0.1 and about 1000 Hz. Fixed repetition rates can also be used between about 0.1 Hz and about 1000 Hz. An induced electric field from about 0.001 mV/cm to about 100 mV/cm is generated. Another embodiment according to the present disclosure comprises an about 0.01 millisecond to an about 10 millisecond burst of high frequency sinusoidal waves, such as 27.12 MHz, repeating at about 1 to about 100 bursts per second. An induced electric field from about 0.001 mV/cm to about 100 mV/cm is generated. Resulting waveforms can be delivered via inductive or capacitive coupling.

It is another object of the present disclosure to provide an electromagnetic method of treatment of hair and other cerebrofacial tissues comprising a broad-band, high spectral density electromagnetic field.

It is a further object of the present disclosure to provide an electromagnetic method of treatment of hair and other cerebrofacial tissues comprising amplitude modulation of a pulse burst envelope of an electromagnetic signal that will induce coupling with a maximum number of relevant EMF-sensitive pathways in cells or tissues.

It is another object of the present disclosure to provide enhanced hair and other cerebrofacial tissue growth and repair in individuals that have experienced hair loss due to medical conditions such as psoriasis, and hair loss as a result of medication shock and usage.

It is another object of the present disclosure to provide an apparatus and method that may be used in conjunction with pharmacological and herbal agents, and in conjunction with standard physical therapy and medical treatments.

It is another object of the present disclosure to provide enhanced hair and other cerebrofacial tissue growth and repair in conjunction with topical and medication treatments.

It is another object of the present disclosure to provide a self-contained hair restoration and cerebrofacial condition apparatus that can be portable, fashionable, and worn whenever and wherever an individual so desires.

It is another object of the present disclosure to provide a self-contained hair restoration and cerebrofacial condition apparatus that can be programmed to release electromagnetic therapy treatment at, at least one of specific and random time intervals.

It is a still further object of the present disclosure to provide a self-contained hair restoration and cerebrofacial condition apparatus for use in any type of headwear, for example a hat, sweatband, and flexible knit cap.

It is yet another object of the present disclosure to increase blood flow to damaged cerebrofacial tissue by modulation of vasodilation and stimulating neovascularization.

It is yet another object of the present disclosure to prevent the loss and deterioration of cells and tissues of any type in the cerebrofacial area.

A further object of the present disclosure is to augment the activity of cells and tissues in the cerebrofacial area.

Yet a further object of the present disclosure is to increase cell population in the cerebrofacial area.

It is yet a further object of the present disclosure to prevent the deterioration of neurons in the cerebrofacial area.

It is yet another object of the present disclosure to increase neuron population in the cerebrofacial area.

It is yet a further object of the present disclosure to prevent the deterioration of adrenergic neurons in the cerebrofacial area.

It is yet another object of the present disclosure to increase adrenergic neuron population in the cerebrofacial area.

It is a yet another object of the present disclosure to provide an apparatus for cerebrofacial conditions that modulates angiogenesis and neovascularization that can be operated at reduced power levels and still possess benefits of safety, economics, portability, and reduced electromagnetic interference.

It is an object of the present disclosure to configure a power spectrum of a waveform by mathematical simulation by using signal to noise ratio (“SNR”) analysis to configure a waveform optimized to modulate angiogenesis and neovascularization in a cerebrofacial area then coupling the configured waveform using a generating device such as ultra-lightweight wire coils that are powered by a waveform configuration device such as miniaturized electronic circuitry.

It is another object of the present disclosure to modulate angiogenesis and neovascularization by evaluating Power SNR for any target pathway structure such as molecules, cells, tissues and organs in a cerebrofacial area using any input waveform, even if electrical equivalents are non-linear as in a Hodgkin-Huxley membrane model.

It is another object of the present disclosure to provide a self-contained hair restoration and cerebrofacial apparatus that incorporates use of Power SNR to regulate and adjust electromagnetic therapy treatment.

It is another object of the present disclosure to provide a method and apparatus for treating hair loss and other cerebrofacial conditions occurring in animals and humans using electromagnetic fields selected by optimizing a power spectrum of a waveform to be applied to a biochemical target pathway structure to enable modulation of angiogenesis and neovascularization within molecules, cells, tissues and organs in a cerebrofacial area.

It is another object of the present disclosure to significantly lower peak amplitudes and shorter pulse duration. This can be accomplished by matching via Power SNR, a frequency range in a signal to frequency response and sensitivity of a target pathway structure such as a molecule, cell, tissue, and organ, in a cerebrofacial area to enable modulation of angiogenesis and neovascularization.

### Part 8

An embodiment according to the present disclosure comprises an electromagnetic signal having a pulse burst envelope of spectral density to efficiently couple to physiologically relevant dielectric pathways, such as cellular membrane receptors, ion binding to cellular enzymes, and general transmembrane potential changes. The use of a burst duration which is generally below 100 microseconds for each PRF burst, limits the frequency components that could couple to the relevant dielectric pathways in cells and tissue. An embodiment according to the present disclosure increases the number of frequency components transmitted to relevant cellular pathways whereby access to a larger range of biophysical phenomena applicable to known healing mechanisms, including enhanced second messenger release, enzyme activity and growth factor and cytokine release can be achieved. By increasing burst duration and applying a random, or other envelope, to the pulse burst envelope of mono-polar or bi-polar rectangular or sinusoidal pulses which induce peak electric fields between 10⁻⁶ and 10 V/cm, a more efficient and greater effect can be achieved on biological healing processes applicable to both soft and hard tissues in humans, animals and plants.

Another embodiment according to the present disclosure comprises known cellular responses to weak external stimuli such as heat, light, sound, ultrasound and electromagnetic fields. Cellular responses to such stimuli result in the production of protective proteins, for example, heat shock proteins, which enhance the ability of the cell, tissue, organ to withstand and respond to such external stimuli. Electromagnetic fields configured according to an embodiment of the present disclosure enhance the release of such compounds thus advantageously providing an improved means to enhance prophylactic protection and wellness of living organisms. In certain ophthalmic diseases there are physiological deficiencies and disease states that can have a lasting and deleterious effect on the proper functioning of the ophthalmic system. Those physiological deficiencies and disease states can be positively affected on a non-invasive basis by the therapeutic application of waveforms configured according to an embodiment of the present disclosure. In addition, electromagnetic waveforms configured according to an embodiment of the present disclosure can have a prophylactic effect on the ophthalmic system whereby a disease condition can be prevented, and if a disease condition already exists in its earliest stages, that condition can be prevented from developing into a more advanced state.

An example of an ophthalmic disease that can be positively affected by an embodiment according to the present disclosure, both on a chronic disease as well on a prophylactic basis, is macular degeneration. Age-related macular degeneration (“ARMD”) is the most common cause of irreversible vision loss those over the age of 60. Macular degeneration is a disorder of the retina, the light-sensitive inner lining of the back of the eye. There are a number of abnormalities associated with the term “age-related macular degeneration.” They range from mild changes with no decrease in vision to abnormalities severe enough to result in the loss of all “straight ahead” vision. Macular degeneration does not cause total blindness because the remaining and undamaged parts of the retina around the macula continue to provide “side” vision.

There are two main types of macular degeneration, “dry” and “wet.” With respect to dry macular degeneration, aging causes the cells in the retina to become less efficient. Deposits of tissue, called drusen, appear under the retina which can be identified through visual examination. A few small drusen may cause no decrease in vision. However, if too many large drusen develop, vision will decrease. The application of electromagnetic waveforms configured according to an embodiment of the present disclosure can positively effect tissue present in the retina and modify the propensity to form drusen, thereby having an effect on the progression of dry macular degeneration. Conversely, wet macular degeneration is a function of leaking of the capillaries in the layer of cells below the retina called the retinal pigment epithelium. Electromagnetic waveforms configured according to an embodiment of the present disclosure, have proven to have a positive effect on circulatory vessels and other tissues which can lead to an improvement in the disease state of wet macular degeneration.

Another advantage of electromagnetic waveforms configured according to an embodiment of the present disclosure is that by applying a high spectral density voltage envelope as the modulating or pulse-burst defining parameter, the power requirement for such increased duration pulse bursts can be significantly lower than that of shorter pulse bursts containing pulses within the same frequency range; this is due to more efficient matching of the frequency components to the relevant cellular/molecular process. Accordingly, the dual advantages, of enhanced transmitted dosimetry to the relevant dielectric pathways and of decreased power requirement are achieved.

The present disclosure relates to a therapeutically beneficial method of and apparatus for non-invasive pulsed electromagnetic treatment for enhanced condition, repair and growth of living tissue in animals, humans and plants. This beneficial method operates to selectively change the bioelectromagnetic environment associated with the cellular and tissue environment through the use of electromagnetic means such as PRF generators and applicator heads. An embodiment of the present disclosure more particularly includes the provision of a flux path, to a selectable body region, of a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter in which the instantaneous minimum amplitude thereof is not smaller than the maximum amplitude thereof by a factor of one ten-thousandth. Further, the repetition rate of such pulse bursts may vary from 0.01 to 10,000 Hz. Additionally, a mathematically-definable parameter can be employed in lieu of said random amplitude envelope of the pulse bursts.

By increasing a range of frequency components transmitted to relevant cellular pathways, access to a large range of biophysical phenomena applicable to known healing mechanisms, including enhanced second messenger release, enzyme activity and growth factor and cytokine release, is advantageously achieved.

Another advantage of an embodiment according to the present disclosure is that by applying a high spectral density voltage envelope as a modulating or pulse-burst defining parameter, power requirements for such modulated pulse bursts can be significantly lower than that of an unmodulated pulse. This is due to more efficient matching of the frequency components to the relevant cellular/molecular process. Accordingly, the dual advantages of enhanced transmitting dosimetry to relevant dielectric pathways and of decreasing power requirements are achieved.

A further object of the present disclosure is to integrate at least one coil delivering a waveform configured by SNR/Power analysis of a target pathway structure, with a therapeutic surface, structure or device to enhance the effectiveness of such therapeutic surface, structure or device to augment the activity of cells and tissues of any type in any living target area.

It is yet a further object of the present disclosure to provide an improved electromagnetic method of the beneficial treatment of living cells and tissue by the modulation of electromagnetically sensitive regulatory processes at the cell membrane and at junctional interfaces between cells.

A further object of the present disclosure is to provide a means for the use of electromagnetic waveforms to cause a beneficial effect in the treatment of ophthalmic diseases.

It is a further object of the present disclosure to provide improved means for the prophylactic treatment of the ophthalmic system to improve function and to prevent or arrest diseases of the ophthalmic system.

It is another object to provide an electromagnetic treatment method of the above type having a broad-band, high spectral density electromagnetic field.

It is a further object of the present disclosure to provide a method of the above type in which amplitude modulation of the pulse burst envelope of the electromagnetic signal will induce coupling with a maximum number of relevant EMF-sensitive pathways in cells or tissues.

It is a still further object of the present disclosure to provide an improved means to enhance second messenger release.

### Part 9

The methods and apparatus according to present disclosure, comprises delivering electromagnetic signals to respiratory target pathway structures, such as respiratory molecules, respiratory cells, respiratory tissues, and respiratory organs for treatment of inflammatory processes leading to excessive fibrous tissue formation such as scar tissue, associated with the inhalation of foreign particles into lung tissue. An embodiment according to the present disclosure utilizes SNR and Power SNR approaches to configure bioeffective waveforms and incorporates miniaturized circuitry and lightweight flexible coils. This advantageously allows a device that utilizes the SNR and Power SNR approaches, miniaturized circuitry, and lightweight flexible coils to be completely portable and if desired to be constructed as disposable.

An embodiment according to the present disclosure comprises an electromagnetic signal having a pulse burst envelope of spectral density to efficiently couple to physiologically relevant dielectric pathways, such as cellular membrane receptors, ion binding to cellular enzymes, and general transmembrane potential changes. The use of a burst duration which is generally below 100 microseconds for each PRF burst, limits the frequency components that could couple to the relevant dielectric pathways in cells and tissue. An embodiment according to the present disclosure increases the number of frequency components transmitted to relevant cellular pathways whereby access to a larger range of biophysical phenomena applicable to known healing mechanisms, including enhanced second messenger release, enzyme activity and growth factor and cytokine release can be achieved. By increasing burst duration and applying a random, or other envelope, to the pulse burst envelope of mono-polar or bi-polar rectangular or sinusoidal pulses which induce peak electric fields between 10.sup.-6 and 10 V/cm, a more efficient and greater effect can be achieved on biological healing processes applicable to both soft and hard tissues in humans, animals and plants.

Another embodiment according to the present disclosure comprises known cellular responses to weak external stimuli such as heat, light, sound, ultrasound and electromagnetic fields. Cellular responses to such stimuli result in the production of protective proteins, for example, heat shock proteins, which enhance the ability of the cell, tissue, organ to withstand and respond to such external stimuli. Electromagnetic fields configured according to an embodiment of the present disclosure enhance the release of such compounds thus advantageously providing an improved means to enhance prophylactic protection and wellness of living organisms. In certain respiratory diseases there are physiological deficiencies and disease states that can have a lasting and deleterious effect on the proper functioning of the respiratory system. Those physiological deficiencies and disease states can be positively affected on a non-invasive basis by the therapeutic application of waveforms configured according to an embodiment of the present disclosure. In addition, electromagnetic waveforms configured according to an embodiment of the present disclosure can have a prophylactic effect on the respiratory system whereby a disease condition can be prevented, and if a disease condition already exists in its earliest stages, that condition can be prevented from developing into a more advanced state.

An example of a respiratory disease that can be positively affected by an embodiment according to the present disclosure, both on a chronic disease as well on a prophylactic basis, is inflammation in lung tissue resulting from inhalation of foreign particles that remain in lung tissue. Electromagnetic waveforms configured according to an embodiment of the present disclosure, have proven to have a positive effect on circulatory vessels and other tissues which can lead to reducing inflammation that can lead to lung disease.

Another advantage of electromagnetic waveforms configured according to an embodiment of the present disclosure is that by applying a high spectral density voltage envelope as the modulating or pulse-burst defining parameter, the power requirement for such increased duration pulse bursts can be significantly lower than that of shorter pulse bursts containing pulses within the same frequency range; this is due to more efficient matching of the frequency components to the relevant cellular/molecular process. Accordingly, the dual advantages, of enhanced transmitted dosimetry to the relevant dielectric pathways and of decreased power requirement are achieved.

The present disclosure relates to a therapeutically beneficial method of and apparatus for non-invasive pulsed electromagnetic treatment for enhanced condition, repair and growth of living tissue in animals, humans and plants. This beneficial method operates to selectively change the bioelectromagnetic environment associated with the cellular and tissue environment through the use of electromagnetic means such as PRF generators and applicator heads. An embodiment of the present disclosure more particularly includes the provision of a flux path, to a selectable body region, of a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter in which the instantaneous minimum amplitude thereof is not smaller than the maximum amplitude thereof by a factor of one ten-thousandth. Further, the repetition rate of such pulse bursts may vary from 0.01 to 10,000 Hz. Additionally, a mathematically-definable parameter can be employed in lieu of said random amplitude envelope of the pulse bursts.

Another advantage of an embodiment according to the present disclosure is that by applying a high spectral density voltage envelope as a modulating or pulse-burst defining parameter, power requirements for such modulated pulse bursts can be significantly lower than that of an unmodulated pulse. This is due to more efficient matching of the frequency components to the relevant cellular/molecular process. Accordingly, the dual advantages of enhanced transmitting dosimetry to relevant dielectric pathways and of decreasing power requirements are achieved.

Specifically, broad spectral density bursts of electromagnetic waveforms, configured to achieve maximum signal power within a bandpass of a biological target, are selectively applied to target pathway structures such as living organs, tissues, cells and molecules. Waveforms are selected using a novel amplitude/power comparison with that of thermal noise in a target pathway structure. Signals comprise bursts of at least one of sinusoidal, rectangular, chaotic and random wave shapes have frequency content in a range of 0.01 Hz to 100 MHz at 1 to 100,000 bursts per second, with a burst duration from 0.01 to 100 milliseconds, and a burst repetition rate from 0.01 to 1000 bursts/second. Peak signal amplitude at a target pathway structure such as tissue, lies in a range of 1 μV/cm to 100 mV/cm. Each signal burst envelope may be a random function providing a means to accommodate different electromagnetic characteristics of healing tissue. Preferably the present disclosure comprises a 20 millisecond pulse burst, repeating at 1 to 10 burst/second and comprising 5 to 200 microsecond symmetrical or asymmetrical pulses repeating at 0.1 to 100 kilohertz within the burst. The burst envelope is a modified 1/f function and is applied at random repetition rates. Fixed repetition rates can also be used between about 0.1 Hz and about 1000 Hz. An induced electric field from about 0.001 mV/cm to about 100 mV/cm is generated. Another embodiment according to the present disclosure comprises a 4 millisecond of high frequency sinusoidal waves, such as 27.12 MHz, repeating at 1 to 100 bursts per second. An induced electric field from about 0.001 mV/cm to about 100 mV/cm is generated. Resulting waveforms can be delivered via inductive or capacitive coupling for 1 to 30 minute treatment sessions delivered according to predefined regimes by which PEMF treatment may be applied for 1 to 12 daily sessions, repeated daily. The treatment regimens for any waveform configured according to the instant disclosure may be fully automated. The number of daily treatments may be programmed to vary on a daily basis according to any predefined protocol.

In another aspect of the present disclosure, an electromagnetic method of treatment of living cells and tissues comprising modulation of electromagnetically sensitive regulatory processes at a cell membrane and at junctional interfaces between cells is provided.

In another aspect of the present disclosure, multiple coils deliver a waveform configured by SNR/Power analysis of a target pathway structure, to increase area of treatment coverage.

In another aspect of the present disclosure, multiple coils that are simultaneously driven or that are sequentially driven such as multiplexed, deliver the same or different optimally configured waveforms as illustrated above.

In still another aspect of the present disclosure, flexible, lightweight coils that focus the EMF signal to the affected tissue delivering a waveform configured by SNR/Power analysis of a target pathway structure, are incorporated into dressings and ergonomic support garments.

In a further aspect of the present disclosure, at least one coil delivering a waveform configured by SNR/Power analysis of a target pathway structure, is integrated with a therapeutic surface, structure or device to enhance the effectiveness of such therapeutic surface, structure or device to augment the activity of cells and tissues of any type in any living target area.

In yet a further aspect of the present disclosure, an improved electromagnetic method of the beneficial treatment of living cells and tissue by the modulation of electromagnetically sensitive regulatory processes at the cell membrane and at junctional interfaces between cells is provided.

In a further aspect of the present disclosure, a means for the use of electromagnetic waveforms to cause a beneficial effect in the treatment of respiratory diseases is provided.

In a further aspect of the present disclosure, improved means for the prophylactic treatment of the respiratory system to improve function and to prevent or arrest diseases of the respiratory system is provided.

In another aspect of the present disclosure, an electromagnetic treatment method of the above type having a broad-band, high spectral density electromagnetic field is provided.

In a further aspect of the present disclosure, a method of the above type in which amplitude modulation of the pulse burst envelope of the electromagnetic signal will induce coupling with a maximum number of relevant EMF-sensitive pathways in cells or tissues is provided.

In another aspect of the present disclosure, an improved method of enhancing soft tissue and hard tissue repair is provided.

In another aspect of the present disclosure, an improved method of increasing blood flow to affected tissues by modulating angiogenesis is provided.

In another aspect of the present disclosure, an improved method of increasing blood flow to enhance the viability and growth or differentiation of implanted cells, tissues and organs is provided.

In another aspect of the present disclosure, an improved method of increasing blood flow in cardiovascular diseases by modulating angiogenesis is provided.

In another aspect of the present disclosure, beneficial physiological effects through improvement of micro-vascular blood perfusion and reduced transudation are provided.

In another aspect of the present disclosure, an improved method of treatment of maladies of the bone and other hard tissue is provided.

In still further aspect of the present disclosure, an improved means of the treatment of edema and swelling of soft tissue is provided.

In a further aspect of the present disclosure, an improved means to enhance second messenger release is provided.

In another aspect of the present disclosure, a means of repair of damaged soft tissue is provided.

In yet another aspect of the present disclosure, a means of increasing blood flow to damaged tissue by modulation of vasodilation and stimulating neovascularization is provided.

In yet a further aspect of the present disclosure, an apparatus that can operate at reduced power levels as compared to those of related methods known in electromedicine and respective biofield technologies, with attendant benefits of safety, economics, portability, and reduced electromagnetic interference is provided.

“About” for purposes of this aspect of the disclosure means a variation of plus or minus 0.1%.

“Respiratory” for purposes of this aspect of the disclosure means any organs and structures such as nose, nasal passages, nasopharynx, larynx, trachea, bronchi, lungs and airways in which gas exchange takes.

### Part 10

The apparatus and method according to present disclosure, comprise delivering electromagnetic signals to fibrous capsule formation and capsular contracture target pathway structures, such as capsular molecules, capsular cells, capsular tissues, and capsular organs for alleviation of the propensity of a capsule to compress or harden, for reduction of excessive fibrous capsule formation, and for reduction in existing capsule involvement with a physical area of a body. An embodiment according to the present disclosure utilizes SNR and Power SNR approaches to configure bioeffective waveforms and incorporates miniaturized circuitry and lightweight flexible coils. This advantageously allows a device that utilizes the SNR and Power SNR approaches, miniaturized circuitry, and lightweight flexible coils to be completely portable and if desired to be constructed as disposable.

An apparatus comprising an electromagnetic signal generating means for emitting signals comprising bursts of at least one of sinusoidal, rectangular, chaotic, and random waveforms, having a frequency content in a range of about 0.01 Hz to about 100 MHz at about 1 to about 100,000 waveforms per second, having a burst duration from about 1 μsec to about 100 msec, and having a burst repetition rate from about 0.01 to about 1000 bursts/second, wherein the waveforms are adapted to have sufficient signal to noise ratio of at least about 0.2 in respect of a given fibrous capsule formation and capsular contracture target pathway structure to modulate at least one of ion and ligand interactions in that fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure, an electromagnetic signal coupling means wherein the coupling means comprises at least one of an inductive coupling means and a capacitive coupling means, connected to the electromagnetic signal generating means for delivering the electromagnetic signal to the fibrous capsule formation and capsular contracture target pathway structure, and a garment wherein the electromagnetic signal generating means and electromagnetic signal coupling means are incorporated into the garment.

An apparatus comprising a waveform configuration means for configuring at least one waveform to have sufficient signal to noise ratio or power signal to noise ratio of at least about 0.2, to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure, a coupling device connected to the waveform configuration means by at least one connecting means for generating an electromagnetic signal from the configured at least one waveform and for coupling the electromagnetic signal to the fibrous capsule formation and capsular contracture target pathway structure whereby the at least one of ion and ligand interactions are modulated, and a garment incorporating the waveform configuration means, the at least one connecting means, and the coupling device.

A method comprising establishing baseline thermal fluctuations in voltage and electrical impedance at a fibrous capsule formation and capsular contracture target pathway structure depending on a state of the fibrous capsule tissue, evaluating a signal to noise ratio by calculating a frequency response of the impedance of the target pathway structure divided by a calculated frequency response of baseline thermal fluctuations in voltage across the target pathway structure, configuring at least one waveform to have sufficient signal to noise ratio of at least about 0.2 to modulate at least one of ion and ligand interactions whereby the at least one of ion and ligand interactions are detectable in the fibrous capsule formation and capsular contracture target pathway structure above the evaluated baseline thermal fluctuations in voltage, generating an electromagnetic signal from the configured at least one waveform; and coupling the electromagnetic signal to the fibrous capsule formation and capsular contracture target pathway structure using a coupling device.

“About” for purposes of this aspect of the disclosure means a variation of plus or minus 50%.

### Part 11

Another aspect of the disclosure relates to methods and apparatuses for treating patients with pulsed electromagnetic therapies (PEMF). The PEMF waveform and the period between PEMF waveform pulses can be configured to simultaneously increase the rate of cGMP production and to minimize the rate of inhibition of cGMP by compounds such as PDE.

In particular, described herein are methods of optimizing PEMF treatment based on the surprising finding that the effectiveness of a non-invasive, relatively low-energy or very low-energy PEMF treatment depends on the ratio of the duration of the treatment interval and the duration of the inter-treatment interval (also referred to herein as the inter-treatment period). The methods described herein are invented from the new finding that the for externally-applied low-energy or (in some variations) very low-energy PEMF treatments, a ratio of treatment interval to inter-treatment period of greater than about 1:6 (e.g., an inter-treatment period that is greater than six times the treatment interval) results in efficacious treatment, whereas ratios less than 1:6 do not. In addition, in some variations it may be beneficial to have ratios of treatment interval to inter-treatment period of less than about 1:100. For example, the ratio of treatment interval to inter-treatment period may be greater than about 1:7, greater than about 1:8, greater than about 1:9, greater than about 1:10, greater than about 1:11, greater than about 1:12, greater than about 1:15, greater than about 1:18, etc., and/or between about 1:6 and 1:1000, between about 1:6 and 1:500, between about 1:6 and 1:100, between about 1:6 and 1:75, between about 1:6 and 1:50, between about 1:8 and 1:1000, between about 1:8 and 1:500, between about 1:8 and 1:100, etc.

The apparatuses described herein may be generally configured to be worn against the body (e.g., incorporated into a garment, jewelry, hat, bed, chair, etc.), and may be specifically adapted/configured to deliver non-invasive, relatively low-energy or very low-energy PEMF treatment in which the ratio of treatment interval to inter-treatment period is as described herein.

For example described herein are methods for treating a patient (e.g., human, animal, etc.) that may generally include: generating a pulsed electromagnetic field (PEMF) from a pulsed electromagnetic field source; applying the pulsed electromagnetic field in proximity to a target region affected by an injury or condition to reduce a physiological response to the injury or condition for a treatment interval that is greater than 10 minutes; discontinuing the application of the pulsed electromagnetic field for an inter-treatment period that is greater than six times the treatment interval; and repeating, for a plurality of times, the steps of generating, applying and discontinuing.

A pulsed electromagnetic field source may include any apparatus, including those described herein or otherwise known in the art, that can be used to apply relatively low-energy PEMF signals. Examples of such devices and PEMF signals (including low-energy PEMF signals) are described, for example, in U.S. patent applications: U.S. Pat. Nos. 7,744,524, 7,740,574, 8,415,123, 7,758,490, 7,896,797 and 8,343,027, and pending applications no.: US-2010-0210893, US-2010-0222631, US-2013-0274540, US-2014-0046115, US-2014-0046117, US-2011-0207989, US-2012-0116149, US-2014-0213843, US-2014-0213844, and US-2012-0089201-A1. Each of these patents and pending applications is herein incorporated by reference in its entirety, and in particular for its teaching of PEMF application devices, waveforms, and therapies.

A relatively low-energy PEMF waveform may generally apply milliTesla (mT), e.g., between about 1 and 100 mT, magnetic field strength, or average magnetic field strength. Very low-energy PEMF may apply microTesla (μT), e.g., less than 1 mT, less than 100 μT, less than 50 μT, less than 20 μT, less than 10 μT, less than 5 μT, etc. The PEMF signal, though relatively or very low energy may be applied in the specific pulsed waveforms as described herein to any target region.

A target region may be any body region, including surface (e.g., skin) or internal (e.g., brain, organ, etc.) region, particularly those that are more superficially located. Wounds such as surgical wounds are an example of a target region. Nerves, including spinal, peripheral or central (e.g., brain) nervous system regions may also be treated as described herein. The treatments described herein may be used to treat a medical disorder, and may modulate or improve a physiological response to the injury or condition, including but not limited to, swelling/inflammation, necrosis, healing (e.g., tissue growth, cell migration), scarring, etc.

In general, a treatment interval may include the period during which treatment (PEMF energy) is actively being applied, for example, as a burst or plurality of bursts of pulses. The waveforms may be applied at a regular, irregular or random duration, period or wave-shape within the burst (envelope) and the amplitude of the envelope may be regular (e.g., sinusoidal, square, etc.), irregular, or random. In particular, sinusoidal pulses at a carrier frequency (e.g., of 27.12 or a harmonic thereof) may be applied within a rectangular envelope that had a burst duration (e.g., greater than 200 microseconds, greater than 300 microseconds, greater than 400 microseconds, greater than 500 microseconds, greater than 600 microseconds, between 500 microseconds and 1 second, etc.), and may be repeated at a burst repetition rate. The treatment interval may therefore include quiescent periods, but they are typically part of the inter-pulse or inter-burst periods defining the periodicity of the waveforms or burst of waveforms. A treatment interval may be between 5 and 600 minutes, but more likely between 5 and 50 minutes. As described herein, it may be particularly efficacious to apply treatment for a treatment interval of greater than about 10 minutes, e.g., greater than about 11, 12, 13, 14, 15, 16, 17, 18, 19, 20, etc. minutes.

During any of the treatments described herein the treatment periods may be divided by off-times (inter-treatment periods) during which no PEMF signals are applied by the apparatus, which may be particularly configured to prevent the application of any PEMF signal during this inter-treatment period.

According to another aspect of the disclosure, the pulsed electromagnetic field is configured to simultaneously increase the rate of ion-dependent signaling and to minimize the rate of inhibition of such signaling by natural compounds. This period may be referred to as a period during which the PEMF signal application is discontinued, e.g., discontinuing the application of the pulsed electromagnetic field for an inter-treatment period (or “off time”). Following the discontinuation, another treatment period (and another inter-treatment period, off-time) may be repeated. The waveforms applied during the subsequent treatment periods may be the same or different. For example, the waveform characteristics, such as amplitude, frequency (e.g. burst frequency and/or pulse frequency and/or carrier wave frequency), duration (burst duration, pulse duration), and/or waveform and/or envelope (burst) shape, may be different between, or in some variations within, subsequent treatment periods. The duration of the subsequent treatment period may be the same or different, though they may still be greater than some minimum treatment period duration (e.g., greater than 10 min, 11 min, 12 min, 13 min, 14 min, 15 min, 20 min, 25 min, 30 min, 40 min, 50 min, 60 min, etc.) and/or less than a maximum treatment period duration (e.g., less than 50 min, 60 min, 70 min, 80 min, 90 min, 2 hrs, 2.5 hrs, 3 hrs, etc.), where the minimum is always less than the maximum. In some variations, the method or apparatus may adjust one or more characteristics of the treatment period and/or the treatment period duration based on feedback measured or otherwise received from the patient, including feedback based on a detected level of a biomarker.

The pulsed electromagnetic field may be configured to simultaneously increase the rate of cGMP signaling and to minimize the rate of inhibition of such signaling by compounds such as phosphodiesterase (PDE). The pulsed electromagnetic field may be configured to simultaneously increase the rate of cGMP signaling and to minimize the rate of inhibition of such signaling by compounds such as phosphodiesterase (PDE), is applied for a duration consistent with the above.

The application treatment may include any appropriate number of repetitions, and may be opend-ended (e.g., stopped manually by the patient and/or a medical professional). For example repeating may comprise repeating for at least 10 times, 11 times, 12 times, 20 times, 30 times, 40 times, 50 times, etc. or for some minimum time interval (e.g., 20 min, 25 min, 30 min, 35 min, 40 min, 50 min, 60 min, 90 min, 2 hrs, 4 hrs, 8 hrs, 12 hrs, 18 hrs, 24 hrs, 36 hrs, 2 days, 3 days, 4 days, 5 days, 7 days, 14 days, etc.).

As mentioned, the pulsed electromagnetic field may consist of a burst having any appropriate relatively or very low PEMF waveform characteristics. For example, the PEMF signal within a treatment period may have a duration of greater than 2 msec of a 27.12 MHz carrier repeating at between 1 and 20 bursts/sec at an amplitude of between 2 and 10 μT. The pulsed electromagnetic field may include a burst having a duration of between 2 and 10 msec of a carrier wave repeating at between 1 and 10 bursts/sec at an amplitude of between 3 and 8 μT.

The length of the first treatment interval and the length of the inter-treatment period are selected to minimize phosphodiesterase (PDE) production in the patient.

Any of these methods may also include monitoring the physiological response; and modifying the pulsed electromagnetic field in response to the monitoring step. For example, they may include monitoring the physiological response; and discontinuing treatment once an acceptable level of the physiological response is reached.

The methods may also include modulating inflammatory cytokines and growth factors at the target region by applying the pulsed electromagnetic field to simultaneously increase the rate of such modulation and to minimize the rate of inhibition of such modulation by natural compounds. These methods may also include accelerating the healing of the target region by applying the pulsed electromagnetic field to simultaneously increase the rate of healing and to minimize the rate of inhibition of such healing.

Applying may include applying the pulsed electromagnetic field in proximity to a target region affected by a neurological injury or condition to reduce a physiological response comprises reducing a concentration of IL-β.

As mentioned above, these methods may be used to treat any appropriate injury or condition, including a neurodegenerative disease, e.g., Parkinson's disease Alzheimer's disease, etc. The injury or condition may be a traumatic brain injury (TBI). The injury or condition may be a post-operative inflammation and pain.

Also described herein are apparatuses configured to perform any of these methods. For example, an apparatus for applying pulsed electromagnetic field (PEMF) energy to a subject may include: a generator unit including a signal generator configured to generate a PEMF waveform having configured to simultaneously increase the rate of ion-dependent signaling and to minimize the rate of inhibition of such signaling; a programmable control unit configured to repeatedly provide a signal to the generator unit corresponding to the PEMF waveform for a treatment interval that is 10 minutes or greater followed immediately by an off time having an inter-treatment period that is greater than six times the treatment interval; and an applicator unit configured to be worn by the subject, wherein the generator unit is configured to power the applicator unit to drive transmission of a PEMF signal from the applicator unit based on the PEMF waveform.

The programmable control unit may be programmed to provide an inter-treatment period that is between six and 100 times the treatment interval. The programmable control unit may be configured to prevent the generation of a pulsed electromagnetic field during the inter-treatment period.

Any of these apparatuses may include a shut off to stop the generator unit during the inter-treatment period.

Any appropriate applicator may be used, particularly flexible loop applicators, which may be bent or shaped, though remain a coil. In some variations, the applicator comprises a first loop configured to provide the PEMF waveform and a second loop configured to provide a second PEMF waveform.

A programmable control unit may be configured to repeatedly provide the same signal to the generator unit corresponding to the PEMF waveform.

### Part 12

The apparatus and methods according to present disclosure, comprise delivering electromagnetic signals to hepatic target pathway structures for the treatment of for treating non-alcoholic steatohepatitis. The method comprises generating a pulsed electromagnetic field from a pulsed magnetic field source, and applying the pulsed electromagnetic field in proximity to the patient's liver.

In one aspect, the method comprises generating and applying bursts of at least one of sinusoidal, rectangular, chaotic, and random waveforms, having a frequency content in a range of about 0.01 Hz to about 100 MHz at about 1 to about 100,000 waveforms per second, having a burst duration from about 1 μsec to about 100 msec, and having a burst repetition rate from about 0.01 to about 1000 bursts/second, wherein the waveforms are adapted to have sufficient signal to noise ratio of at least about 0.2 in respect of a given hepatic target pathway structure to modulate at least one of ion and ligand interactions in that hepatic target pathway structure, wherein the signal to noise ratio is evaluated by calculating a frequency response of the impedance of the target path structure divided by a calculated frequency response of baseline thermal fluctuations in voltage across the target path structure.

In another aspect. the method comprises generating PEMF signal comprising 2 msec bursts of a 27.12 MHz sinusoidal wave at a frequency of 2 bursts per second, and applying the signal to a target structure in the patient's liver. The signal induces a magnetic field having a strength of 5 μT Gauss in the target structure.

The apparatus comprises an electromagnetic signal generating means and an electromagnetic signal coupling means. In one aspect, the electromagnetic signal generating means is configured to emit signals comprising bursts of at least one of sinusoidal, rectangular, chaotic, and random waveforms, having a frequency content in a range of about 0.01 Hz to about 100 MHz at about 1 to about 100,000 waveforms per second, having a burst duration from about 1 usec to about 100 msec, and having a burst repetition rate from about 0.01 to about 1000 bursts/second. The electromagnetic signal coupling means comprises at least one of an inductive coupling means and a capacitive coupling means, connected to the electromagnetic signal generating means for delivering the electromagnetic signal to hepatic target pathway structure. In one aspect, the electromagnetic signal generating means and the electromagnetic signal coupling means are integrated into or attached to a therapeutic device to be placed near a patient's liver. The therapeutic device may be in the form of an anatomical support, an anatomical wrap, apparel, a mattress, a mattress pad, a wheelchair, a therapeutic chair, a therapeutic bed, or sporting goods.

In another aspect, the apparatus may comprise low-power, and lightweight electromagnetic treatment device for treating the liver by stimulation of a hepatic pathway structure, the device comprising: a lightweight applicator comprising a flexible wire loop, wherein the applicator is configured to deliver an electromagnetic signal to hepatic tissue; and a control circuit including a micro-controller configured to control the burst duration, the burst period, and the duration of a single treatment application, wherein the micro-controller is configured to apply a signal to the applicator to induce an electric field of amplitude of between about 1 μV/cm to about 100 mV/cm at the hepatic tissue and a peak induced magnetic field between about 1 μT and about 20 μT, wherein the control circuit generates a burst of waveforms having a burst duration of greater than 0.5 msec and a burst period of between about 0.1 to about 10 seconds to produce a signal that is above background electrical activity in the hepatic tissue.

The above and yet other aspects and advantages of the present disclosure will become apparent from the hereinafter set forth Brief Description of the Drawings and Detailed Description of the Invention.

### BRIEF DESCRIPTION OF THE DRAWINGS

FIG. 1 is a flow diagram of a method for electromagnetic treatment of plant, animal, and human target pathway structures such as tissue, organs, cells, and molecules according to an embodiment of the present invention;

FIG. 2 is a view of control circuitry and electrical coils applied to a knee joint according to a preferred embodiment of the present invention;

FIG. 3 is a block diagram of miniaturized circuitry according to a preferred embodiment of the present invention;

FIG. 4A is a line drawing of a wire coil such as an inductor according to a preferred embodiment of the present invention;

FIG. 4B is a line drawing of a flexible magnetic wire according to a preferred embodiment of the present invention;

FIG. 5 depicts a waveform delivered to a target pathway structure such as a molecule, cell, tissue or organ according to a preferred embodiment of the present invention;

FIG. 6 is a view of a positioning device such as a wrist support according to a preferred embodiment of the present invention;

FIG. 7 is a graph illustrating maximally increased myosin phosphorylation for a PMRF signal configured according to an embodiment of the present invention

FIG. 8 is a graph illustrating a power consumption comparison between a 60 Hz signal and a PEMF signal configured according to an embodiment of the present invention.

FIG. 9 is a flow diagram of a method for using an electromagnetic treatment inductive apparatus according to an embodiment of the present invention;

FIG. 10 is a view of control circuitry according to a preferred embodiment of the present invention;

FIG. 11 depicts an electromagnetic treatment inductive apparatus integrated into a hip, thigh, and lower back support garment according to a preferred embodiment of the present invention;

FIG. 12 depicts an electromagnetic treatment inductive apparatus integrated into a head and face support garment according to a preferred embodiment of the present invention;

FIG. 13 depicts an electromagnetic treatment inductive apparatus integrated into a surgical dressing on a human forearm according to a preferred embodiment of the present invention;

FIG. 14 depicts an electromagnetic treatment inductive apparatus integrated into a mattress pad according to a preferred embodiment of the present invention;

FIG. 15A depicts an electromagnetic treatment inductive apparatus integrated into a sock according to a preferred embodiment of the present invention;

FIG. 15B depicts an electromagnetic treatment inductive apparatus integrated into a shoe according to a preferred embodiment of the present invention;

FIG. 16 depicts an electromagnetic treatment inductive apparatus integrated into a therapeutic bed according to a preferred embodiment of the present invention;

FIG. 17 depicts an electromagnetic treatment inductive apparatus integrated into a chest garment according to a preferred embodiment of the present invention.

FIG. 18 is a flow diagram of an electromagnetic treatment method for angiogenesis modulation of living tissues and cells according to an embodiment of the present invention;

FIG. 19 is a flow diagram of a method for accelerating wound repair in living tissues, cells and molecules according to an embodiment of the present invention;

FIG. 20 is a flow diagram of a method for enhancing effectiveness of pharmacological, chemical, cosmetic and topical agents used to treat living tissues, cells and molecules according to an embodiment of the present invention;

FIG. 21 is a graph illustrating an increase in skin blood perfusion achieved according to an embodiment of the present invention.

FIG. 22 is a flow diagram of a electromagnetic therapeutic treatment method for using coils integrated into a positioning device according to an embodiment of the present invention;

FIG. 23 is a bar graph illustrating PMF pre-treatment results;

FIG. 24 is a bar graph illustrating specific PMF signal results;

FIG. 25 is a bar graph illustrating chronic PMF results.

FIG. 26 is a flow diagram of a electromagnetic treatment method for hair restoration and cerebrofacial conditions according to an embodiment of the present invention;

FIG. 27 is a view of an electromagnetic treatment apparatus for hair restoration and cerebrofacial conditions according to a preferred embodiment of the present invention;

FIG. 28 is a flow diagram of a electromagnetic treatment method for treatment of the ophthalmic tissue area according to an embodiment of the present invention;

FIG. 29 is a view of an electromagnetic treatment apparatus for ophthalmic tissue treatment according to a preferred embodiment of the present invention;

FIG. 30 is a block diagram of miniaturized circuitry according to one embodiment of the present invention;

FIG. 31 is a flow diagram of a method for altering an electromagnetic environment of respiratory tissue according to an embodiment of the present invention;

FIG. 32 is a view of an electromagnetic apparatus for respiratory tissue treatment according to an embodiment of the present invention;

FIG. 33 is a view of inductors placed in a vest according to an embodiment of the present invention;

FIG. 34 is a flow diagram of a method for altering fibrous capsule formation and capsular contracture according to an embodiment of the present invention;

FIG. 35 is a view of an electromagnetic apparatus for treating fibrous capsule formation and capsular contracture according to an embodiment of the present invention;

FIG. 36A is a schematic representation of the biological EMF transduction pathway which is a representative target pathway of EMF signals configured according to embodiments described herein;

FIG. 36B is a flow diagram of a method for treating a patient according to an embodiment of the devices and methods described herein;

FIG. 37A is a block diagram of miniaturized circuitry for use with a coil applicator according to some embodiments described;

FIG. 37B illustrates a waveform delivered to a target pathway structure of a plant, animal or human, such as a molecule cell, tissue, organ, or partial or entire organism, according to some embodiments described;

FIG. 38 depicts a disposable dual coil PEMF device used in some of the embodiments described herein. The disposable dual coil PEMF includes a battery-powered signal generator is at the bottom between the coils. A nonthermal pulse-modulated radio frequency PEMF signal, configured to modulate NO signaling, may be delivered to tissue with a preprogrammed dosing regimen for each cohort;

FIG. 39 is a graph showing the effect of different PEMF therapy regimens on the rate of pain reduction following breast reduction surgery in accordance with various embodiments of pain treatments.

FIG. 40 is a graph showing the effect of different PEMF therapy regimens in accordance with the embodiments described herein on pain medication usage of patients after surgery.

FIGS. 41A and 41B are graphs showing the effect of signal configuration on NO production from challenged fibroblasts and chondrocytes (respectively) in culture.

FIG. 42 is a graph showing the effect of exposure time of a given signal configuration on cGMP production in primary neuronal cells.

FIG. 43 is a flow diagram of a method for treating a patient having non-alcoholic steatohepatitis according to an embodiment of the present invention.

FIG. 44 is a perspective view of a test apparatus used to study the efficacy of the method of FIG. 43.

FIG. 45 is a line graph showing changes in weight in mice treated with different regimes for the treatment of non-alcoholic steatohepatitis.

FIG. 46 is a bar graph showing glucose readings for mice treated with different regimes for the treatment of non-alcoholic steatohepatitis.

FIG. 47 is a bar graph showing the liver weights of mice treated with different regimes for the treatment of non-alcoholic steatohepatitis.

### DETAILED DESCRIPTION OF THE PREFERRED EMBODIMENTS

Induced time-varying currents from PEMF or PRF devices flow in a target pathway structure such as a molecule, cell, tissue, and organ, and it is these currents that are a stimulus to which cells and tissues can react in a physiologically meaningful manner The electrical properties of a target pathway structure affect levels and distributions of induced current. Molecules, cells, tissue, and organs are all in an induced current pathway such as cells in a gap junction contact. Ion or ligand interactions at binding sites on macromolecules that may reside on a membrane surface are voltage dependent processes, that is electrochemical, that can respond to an induced electromagnetic field (“E”). Induced current arrives at these sites via a surrounding ionic medium. The presence of cells in a current pathway causes an induced current (“J”) to decay more rapidly with time (“J(t)”). This is due to an added electrical impedance of cells from membrane capacitance and time constants of binding and other voltage sensitive membrane processes such as membrane transport.

Equivalent electrical circuit models representing various membrane and charged interface configurations have been derived. For example, in Calcium (“Ca²⁺”) binding, the change in concentration of bound Ca²⁺ at a binding site due to induced E may be described in a frequency domain by an impedance expression such as:

Z_{b} (ω)=Rᵢₒₙ+1(/iωCᵢₒₙ)

which has the form of a series resistance-capacitance electrical equivalent circuit. Where ω is angular frequency defined as 2πf, where f is frequency, i=−1^{1/2}, Z_{b}(ω) is the binding impedance, and Rᵢₒₙ and Cᵢₒₙ are equivalent binding resistance and capacitance of an ion binding pathway. The value of the equivalent binding time constant, τᵢₒₙ=Rᵢₒₙ Cᵢₒₙ, is related to an ion binding rate constant, k_{b}, via τᵢₒₙ=RionCᵢₒₙ=1/k._{b}. Thus, the characteristic time constant of this pathway is determined by ion binding kinetics.

Induced E from a PEMF or PRF signal can cause current to flow into an ion binding pathway and affect the number of Ca.sup.2+ ions bound per unit time. An electrical equivalent of this is a change in voltage across the equivalent binding capacitance C.sub.ion, which is a direct measure of the change in electrical charge stored by C.sub.ion. Electrical charge is directly proportional to a surface concentration of Ca.sup.2+ ions in the binding site, that is storage of charge is equivalent to storage of ions or other charged species on cell surfaces and junctions. Electrical impedance measurements, as well as direct kinetic analyses of binding rate constants, provide values for time constants necessary for configuration of a PMF waveform to match a bandpass of target pathway structures. This allows for a required range of frequencies for any given induced E waveform for optimal coupling to target impedance, such as bandpass.

Ion binding to regulatory molecules is a frequent EMF target, for example Ca.sup.2+ binding to calmodulin (“CaM”). Use of this pathway is based upon acceleration of wound repair, for example bone repair, that involves modulation of growth factors released in various stages of repair. Growth factors such as platelet derived growth factor (“PDGF”), fibroblast growth factor (“FGF”), and epidermal growth factor (“EGF”) are all involved at an appropriate stage of healing. Angiogenesis is also integral to wound repair and modulated by PMF. All of these factors are Ca/CaM-dependent.

Utilizing a Ca/CaM pathway a waveform can be configured for which induced power is sufficiently above background thermal noise pl conditions, this waveform can have a physiologically significant bioeffect.

Application of a Power SNR model to Ca/CaM requires knowledge of electrical equivalents of Ca²+ binding kinetics at CaM. Within first order binding kinetics, changes in concentration of bound Ca²+ at CaM binding sites over time may be characterized in a frequency domain by an equivalent binding time constant, τᵢₒₙ=RᵢₒₙCᵢₒₙ, where Rᵢₒₙ and Cᵢₒₙ are equivalent binding resistance and capacitance of the ion binding pathway. τᵢₒₙ is related to a ion binding rate constant, k_{b}, via τᵢₒₙ=1/k_{b}. Published values for k_{b} can then be employed in a cell array model to evaluate SNR by comparing voltage induced by a PRF signal to thermal fluctuations in voltage at a CaM binding site. Employing numerical values for PMF response, such as Vₘₐₓ=6.5×10⁷ sec⁻¹, =2.5 μM, K_{D}=30 μM, [Ca²⁺CaM]=K_{D}(+[CaM]), yields k_{b}=665 sec⁻¹=1.5 msec). Such a value for τᵢₒₙ can be employed in an electrical equivalent circuit for ion binding while power SNR analysis can be performed for any waveform structure.

According to an embodiment of the present invention a mathematical model can be configured to assimilate that thermal noise is present in all voltage dependent processes and represents a minimum threshold requirement to establish adequate SNR. Power spectral density, Sₙ(ω), of thermal noise can be expressed as:

Sₙ(ω)=4kTRe└Z_{M}(x,ω)┘

where Z.sub.M(x, ω) is electrical impedance of a target pathway structure, x is a dimension of a target pathway structure and Re denotes a real part of impedance of a target pathway structure. Z_{M}(x, ω) can be expressed as:

Z_{M}(x,ω)=[Rₑ+Rᵢ+R_{g})/y]/tan h(yx).

This equation clearly shows that electrical impedance of the target pathway structure, and contributions from extracellular fluid resistance (“Rₑ”), intracellular fluid resistance (“Rᵢ”) and intermembrane resistance (“R_{g}”) which are electrically connected to a target pathway structure, all contribute to noise filtering.

A typical approach to evaluation of SNR uses a single value of a root mean square (RMS) noise voltage. This is calculated by taking a square root of an integration of Sₙ(ω)=4kT Re[Zₘ(x, ω] over all frequencies relevant to either complete membrane response, or to bandwidth of a target pathway structure. SNR can be expressed by a ratio: SNR=|V_{M}(ω)|/RMS where |V_{M}(ω)| is maximum amplitude of voltage at each frequency as delivered by a chosen waveform to the target pathway structure.

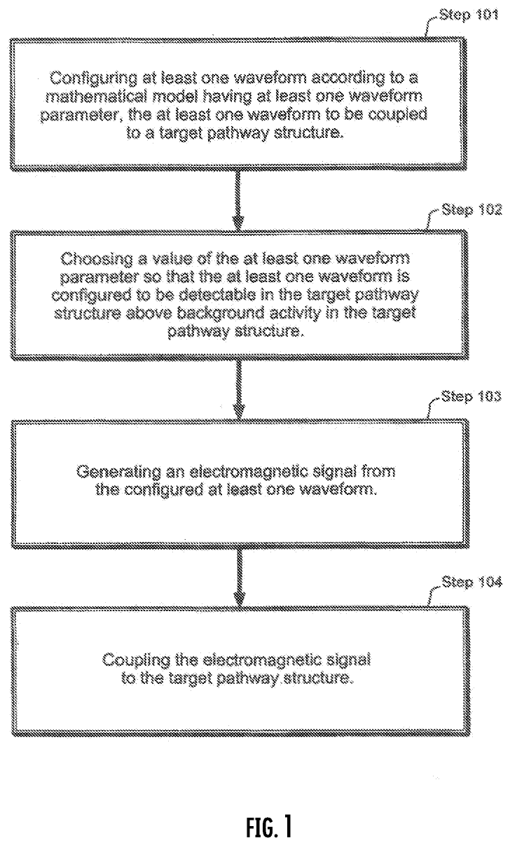

Referring to FIG. 1, wherein FIG. 1 is a flow diagram of a method for delivering electromagnetic signals to target pathway structures such as molecules, cells, tissue and organs of plants, animals, and humans for therapeutic and prophylactic purposes according to an embodiment of the present invention. A mathematical model having at least one waveform parameter is applied to configure at least one waveform to be coupled to a target pathway structure such as a molecule, cell, tissue, and organ (Step 101). The configured waveform satisfies a SNR or Power SNR model so that for a given and known target pathway structure it is possible to choose at least one waveform parameter so that a waveform is detectable in the target pathway structure above its background activity (Step 102) such as baseline thermal fluctuations in voltage and electrical impedance at a target pathway structure that depend upon a state of a cell and tissue, that is whether the state is at least one of resting, growing, replacing, and responding to injury. A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model (Step 102). A repetitive electromagnetic signal can be generated for example inductively or capacitively, from said configured at least one waveform (Step 103). The electromagnetic signal is coupled to a target pathway structure such as a molecule, cell, tissue, and organ by output of a coupling device such as an electrode or an inductor, placed in close proximity to the target pathway structure (Step 104). The coupling enhances a stimulus to which cells and tissues react in a physiologically meaningful manner

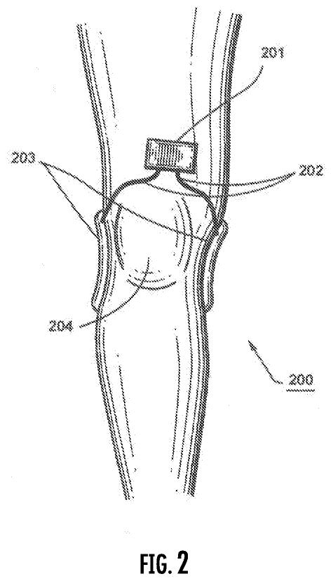

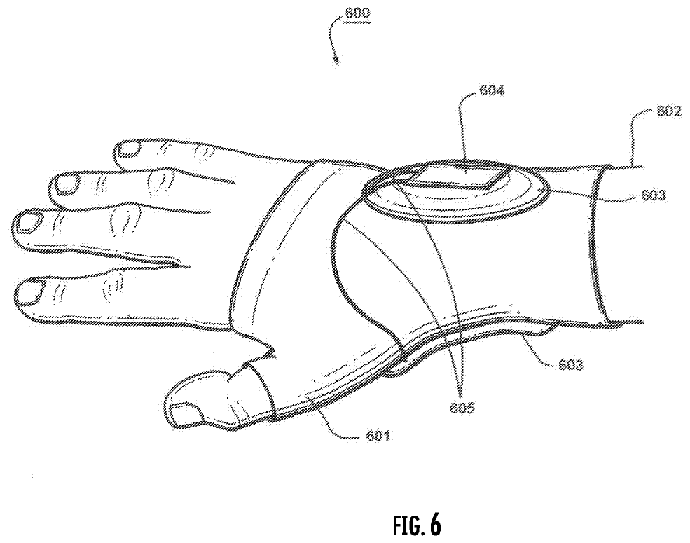

FIG. 2 illustrates a preferred embodiment of an apparatus according to the present invention. A miniature control circuit 201 is coupled to an end of at least one connector 202 such as wire. The opposite end of the at least one connector is coupled to a generating device such as a pair of electrical coils 203. The miniature control circuit 201 is constructed in a manner that applies a mathematical model that is used to configure waveforms. The configured waveforms have to satisfy a SNR or Power SNR model so that for a given and known target pathway structure, it is possible to choose waveform parameters that satisfy SNR or Power SNR so that a waveform is detectable in the target pathway structure above its background activity. A preferred embodiment according to the present invention applies a mathematical model to induce a time-varying magnetic field and a time-varying electric field in a target pathway structure such as a molecule, cell, tissue, and organ, comprising about 10 to about 100 msec bursts of about 1 to about 100 microsecond rectangular pulses repeating at about 0.1 to about 10 pulses per second. Peak amplitude of the induced electric field is between about 1 uV/cm and about 100 mV/cm, varied according to a modified 1/f function where f=frequency. A waveform configured using a preferred embodiment according to the present invention may be applied to a target pathway structure such as a molecule, cell, tissue, and organ for a preferred total exposure time of under 1 minute to 240 minutes daily. However other exposure times can be used. Waveforms configured by the miniature control circuit 201 are directed to a generating device 203 such as electrical coils via connector 202. The generating device 203 delivers a pulsing magnetic field configured according to a mathematical model, that can be used to provide treatment to a target pathway structure such as knee joint 204. The miniature control circuit applies a pulsing magnetic field for a prescribed time and can automatically repeat applying the pulsing magnetic field for as many applications as are needed in a given time period, for example 10 times a day. A preferred embodiment according to the present invention can be positioned to treat the knee joint 204 by a positioning device. The positioning device can be portable such as an anatomical support, and is further described below with reference to FIG. 6. Coupling a pulsing magnetic field to a target pathway structure such as a molecule, cell, tissue, and organ, therapeutically and prophylactically reduces inflammation thereby reducing pain and promotes healing. When electrical coils are used as the generating device 203, the electrical coils can be powered with a time varying magnetic field that induces a time varying electric field in a target pathway structure according to Faraday's law. An electromagnetic signal generated by the generating device 203 can also be applied using electrochemical coupling, wherein electrodes are in direct contact with skin or another outer electrically conductive boundary of a target pathway structure. In yet another embodiment according to the present invention, the electromagnetic signal generated by the generating device 203 can also be applied using electrostatic coupling wherein an air gap exists between a generating device 203 such as an electrode and a target pathway structure such as a molecule, cell, tissue, and organ. An advantage of the preferred embodiment according to the present invention is that its ultra-lightweight coils and miniaturized circuitry allow for use with common physical therapy treatment modalities and at any body location for which pain relief and healing is desired. An advantageous result of application of the preferred embodiment according to the present invention is that a living organism's wellbeing can be maintained and enhanced.

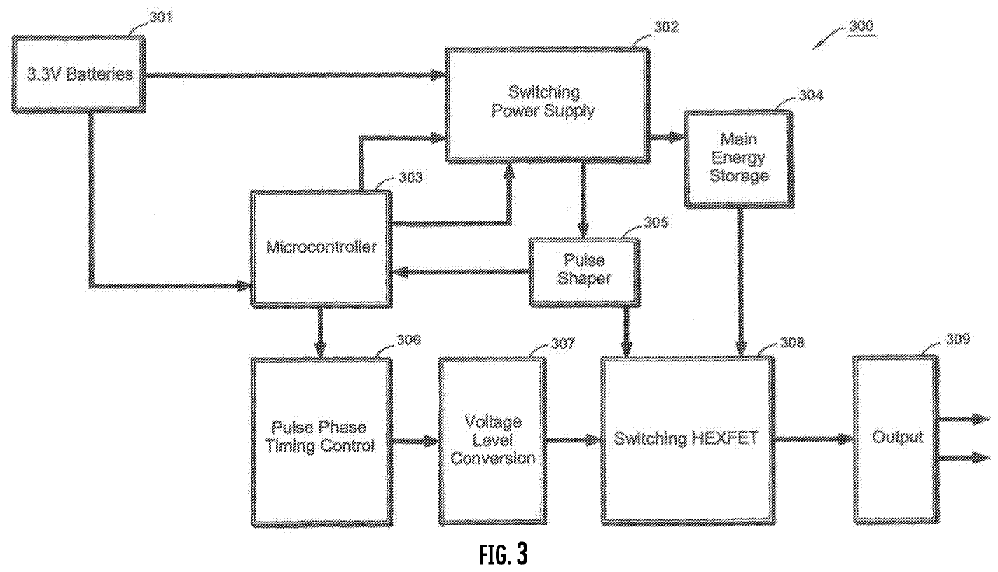

FIG. 3 depicts a block diagram of a preferred embodiment according to the present invention of a miniature control circuit 300. The miniature control circuit 300 produces waveforms that drive a generating device such as wire coils described above in FIG. 2. The miniature control circuit can be activated by any activation means such as an on/off switch. The miniature control circuit 300 has a power source such as a lithium battery 301. A preferred embodiment of the power source has an output voltage of 3.3 V but other voltages can be used. In another embodiment according to the present invention the power source can be an external power source such as an electric current outlet such as an AC/DC outlet, coupled to the present invention for example by a plug and wire. A switching power supply 302 controls voltage to a micro-controller 303. A preferred embodiment of the micro-controller 303 uses an 8 bit 4 MHz micro-controller 303 but other bit MHz combination micro-controllers may be used. The switching power supply 302 also delivers current to storage capacitors 304. A preferred embodiment of the present invention uses storage capacitors having a 220 uF output but other outputs can be used. The storage capacitors 304 allow high frequency pulses to be delivered to a coupling device such as inductors. The micro-controller 303 also controls a pulse shaper 305 and a pulse phase timing control 306. The pulse shaper 305 and pulse phase timing control 306 determine pulse shape, burst width, burst envelope shape, and burst repetition rate. An integral waveform generator, such as a sine wave or arbitrary number generator can also be incorporated to provide specific waveforms. A voltage level conversion sub-circuit 307 controls an induced field delivered to a target pathway structure. A switching Hexfet 308 allows pulses of randomized amplitude to be delivered to output 309 that routes a waveform to at least one coupling device such as an inductor. The micro-controller 303 can also control total exposure time of a single treatment of a target pathway structure such as a molecule, cell, tissue, and organ. The miniature control circuit 300 can be constructed to be programmable and to apply a pulsing magnetic field for a prescribed time and to automatically repeat applying the pulsing magnetic field for as many applications as are needed in a given time period, for example 10 times a day. A preferred embodiment according to the present invention uses treatments times of about 10 minutes to about 30 minutes.

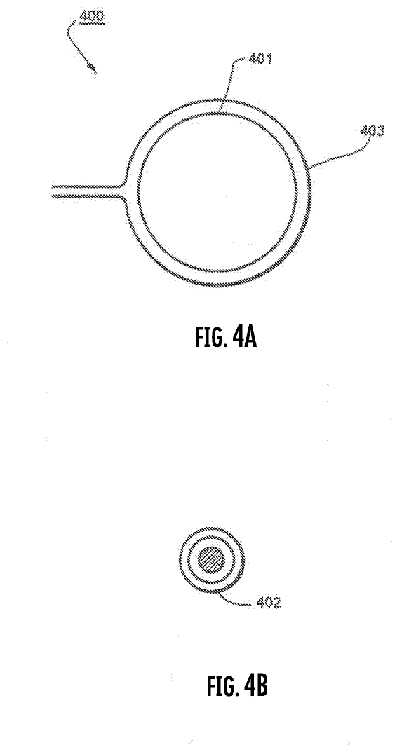

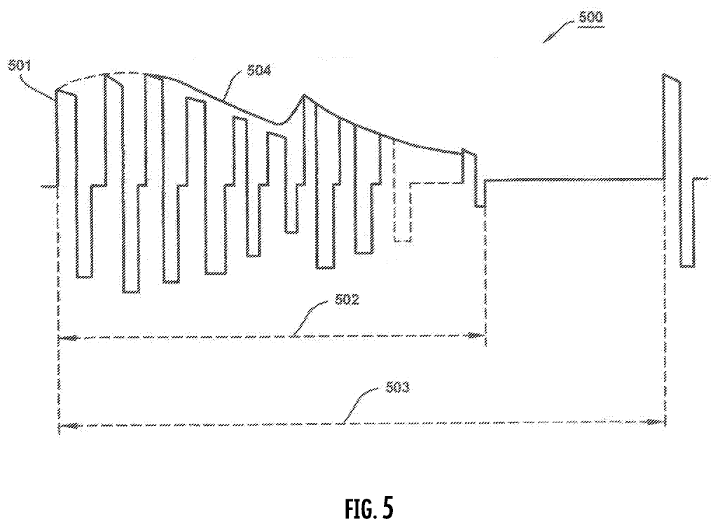

Referring to FIGS. 4A and 4B a preferred embodiment according to the present invention of a coupling device 400 such as an inductor is shown. The coupling device 400 can be an electric coil 401 wound with multistrand flexible magnetic wire 402. The multistrand flexible magnetic wire 402 enables the electric coil 401 to conform to specific anatomical configurations such as a limb or joint of a human or animal. A preferred embodiment of the electric coil 401 comprises about 10 to about 50 turns of about 0.01 mm to about 0.1 mm diameter multistrand magnet wire wound on an initially circular form having an outer diameter between about 2.5 cm and about 50 cm but other numbers of turns and wire diameters can be used. A preferred embodiment of the electric coil 401 can be encased with a non-toxic PVC mould 403 but other non-toxic moulds can also be used.

Referring to FIG. 5 an embodiment according to the present invention of a waveform 500 is illustrated. A pulse 501 is repeated within a burst 502 that has a finite duration 503, alternatively referred to as width 503. The duration 503 is such that a duty cycle which can be defined as a ratio of burst duration to signal period is between about 1 to about 10⁻⁵. A preferred embodiment according to the present invention utilizes pseudo rectangular 10 microsecond pulses for pulse 501 applied in a burst 502 for about 10 to about 50 msec having a modified 1/f amplitude envelope 504 and with a finite duration 503 corresponding to a burst period of between about 0.1 and about 10 seconds, but other waveforms, envelopes, and burst periods that follow a mathematical model such as SNR and Power SNR, may be used.

FIG. 6 illustrates a preferred embodiment according to the present invention of a positioning device such as a wrist support. A positioning device 600 such as a wrist support 601 is worn on a human wrist 602. The positioning device can be constructed to be portable, can be constructed to be disposable, and can be constructed to be implantable. The positioning device can be used in combination with the present invention in a plurality of ways, for example incorporating the present invention into the positioning device for example by stitching, affixing the present invention onto the positioning device for example by Velcro®, and holding the present invention in place by constructing the positioning device to be elastic.

In another embodiment according to the present invention, the present invention can be constructed as a stand-alone device of any size with or without a positioning device, to be used anywhere for example at home, at a clinic, at a treatment center, and outdoors. The wrist support 601 can be made with any anatomical and support material, such as neoprene. Coils 603 are integrated into the wrist support 601 such that a signal configured according to the present invention, for example the waveform depicted in FIG. 5, is applied from a dorsal portion that is the top of the wrist to a plantar portion that is the bottom of the wrist. Micro-circuitry 604 is attached to the exterior of the wrist support 601 using a fastening device such as Velcro® (not shown). The micro-circuitry is coupled to one end of at least one connecting device such as a flexible wire 605. The other end of the at least one connecting device is coupled to the coils 603.

Other embodiments according to the present invention of the positioning device include knee, elbow, lower back, shoulder, other anatomical wraps, and apparel such as garments, fashion accessories, and footwear.

### EXAMPLE 1

The Power SNR approach for PMF signal configuration has been tested experimentally on calcium dependent myosin phosphorylation in a standard enzyme assay. The cell-free reaction mixture was chosen for phosphorylation rate to be linear in time for several minutes, and for sub-saturation Ca²⁺ concentration. This opens the biological window for Ca^{2+/}CaM to be EMF-sensitive. This system is not responsive to PMF at levels utilized in this study if Ca²⁺ is at saturation levels with respect to CaM, and reaction is not slowed to a minute time range. Experiments were performed using myosin light chain (“MLC”) and myosin light chain kinase (“MLCK”) isolated from turkey gizzard. A reaction mixture consisted of a basic solution containing 40 mM Hepes buffer, pH 7.0; 0.5 mM magnesium acetate; 1 mg/ml bovine serum albumin, 0.1% (w/v) Tween 80; and 1 mM EGTA12. Free Ca²⁺ was varied in the 1-7 μM range. Once Ca²⁺ buffering was established, freshly prepared 70 nM CaM, 160 nM MLC and 2 nM MLCK were added to the basic solution to form a final reaction mixture. The low MLC/MLCK ratio allowed linear time behavior in the minute time range. This provided reproducible enzyme activities and minimized pipetting time errors.

The reaction mixture was freshly prepared daily for each series of experiments and was aliquoted in 100 μL portions into 1.5 ml Eppendorf tubes. All Eppendorf tubes containing reaction mixture were kept at 0° C. then transferred to a specially designed water bath maintained at 37±0.1° C. by constant perfusion of water prewarmed by passage through a Fisher Scientific model 900 heat exchanger. Temperature was monitored with a thermistor probe such as a Cole-Parmer model 8110-20, immersed in one Eppendorf tube during all experiments. Reaction was initiated with 2.5 μM 32P ATP, and was stopped with Laemmli Sample Buffer solution containing 30 μM EDTA. A minimum of five blank samples were counted in each experiment. Blanks comprised a total assay mixture minus one of the active components Ca²⁺, CaM, MLC or MLCK. Experiments for which blank counts were higher than 300 cpm were rejected. Phosphorylation was allowed to proceed for 5 min and was evaluated by counting 32P incorporated in MLC using a TM Analytic model 5303 Mark V liquid scintillation counter.

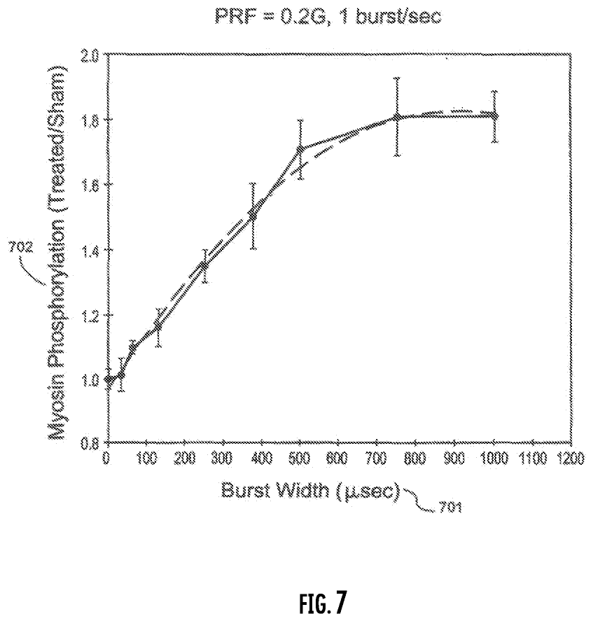

The signal comprised repetitive bursts of a high frequency waveform. Amplitude was maintained constant at 0.2 G and repetition rate was 1 burst/sec for all exposures. Burst duration varied from 65 μsec to 1000 μsec based upon projections of Power SNR analysis which showed that optimal Power SNR would be achieved as burst duration approached 500 μsec. The results are shown in FIG. 7 wherein burst width 701 in μsec is plotted on the x-axis and Myosin Phosphorylation 702 as treated/sham is plotted on the y-axis. It can be seen that the PMF effect on Ca²⁺ binding to CaM approaches its maximum at approximately 500 μsec, just as illustrated by the Power SNR model.

These results confirm that a PMF signal, configured according to an embodiment of the present invention, would maximally increase myosin phosphorylation for burst durations sufficient to achieve optimal Power SNR for a given magnetic field amplitude.

### EXAMPLE 2

According to an embodiment of the present invention use of a Power SNR model was further verified in an in vivo wound repair model. A rat wound model has been well characterized both biomechanically and biochemically, and was used in this study. Healthy, young adult male Sprague Dawley rats weighing more than 300 grams were utilized.

The animals were anesthetized with an intraperitoneal dose of Ketamine 75 mg/kg and Medetomidine 0.5 mg/kg. After adequate anesthesia had been achieved, the dorsum was shaved, prepped with a dilute betadine/alcohol solution, and draped using sterile technique. Using a #10 scalpel, an 8-cm linear incision was performed through the skin down to the fascia on the dorsum of each rat. The wound edges were bluntly dissected to break any remaining dermal fibers, leaving an open wound approximately 4 cm in diameter. Hemostasis was obtained with applied pressure to avoid any damage to the skin edges. The skin edges were then closed with a 4-0 Ethilon running suture. Post-operatively, the animals received Buprenorphine 0.1-0.5 mg/kg, intraperitoneal. They were placed in individual cages and received food and water ad libitum.

PMF exposure comprised two pulsed radio frequency waveforms. The first was a standard clinical PRF signal comprising a 65 μsec burst of 27.12 MHz sinusoidal waves at 1 Gauss amplitude and repeating at 600 bursts/sec. The second was a PRF signal reconfigured according to an embodiment of the present invention. For this signal burst duration was increased to 2000 μsec and the amplitude and repetition rate were reduced to 0.2 G and 5 bursts/sec respectively. PRF was applied for 30 minutes twice daily.

Tensile strength was performed immediately after wound excision. Two 1 cm width strips of skin were transected perpendicular to the scar from each sample and used to measure the tensile strength in kg/mm². The strips were excised from the same area in each rat to assure consistency of measurement. The strips were then mounted on a tensiometer. The strips were loaded at 10 mm/min and the maximum force generated before the wound pulled apart was recorded. The final tensile strength for comparison was determined by taking the average of the maximum load in kilograms per mm² of the two strips from the same wound.

The results showed average tensile strength for the 65 μsec 1 Gauss PRF signal was 19.3±.4.3 kg/mm² for the exposed group versus 13.0.±.3.5 kg/mm² for the control group (p<0.01), which is a 48% increase. In contrast, the average tensile strength for the 2000 μsec 0.2 Gauss PRF signal, configured according to an embodiment of the present invention using a Power SNR model was 21.2±5.6 kg/mm sup.2 for the treated group versus 13.7±4.1 kg/mm sup.2 (p<0.01) for the control group, which is a 54% increase. The results for the two signals were not significantly different from each other.

These results demonstrate that an embodiment of the present invention allowed a new PRF signal to be configured that could be produced with significantly lower power. The PRF signal configured according to an embodiment of the present invention, accelerated wound repair in the rat model in a low power manner versus that for a clinical PRF signal which accelerated wound repair but required more than two orders of magnitude more power to produce.

### EXAMPLE 3

In this example Jurkat cells react to PMF stimulation of a T-cell receptor with cell cycle arrest and thus behave like normal T-lymphocytes stimulated by antigens at the T-cell receptor such as anti-CD3. For example in bone healing, results have shown both 60 Hz and PEMF fields decrease DNA synthesis of Jurkat cells, as is expected since PMF interacts with the T-cell receptor in the absence of a costimulatory signal. This is consistent with an anti-inflammatory response, as has been observed in clinical applications of PMF stimuli. The PEMF signal is more effective. A dosimetry analysis performed according to an embodiment of the present invention demonstrates why both signals are effective and why PEMF signals have a greater effect than 60 Hz signals on Jurkat cells in the most EMF-sensitive growth stage.

Comparison of dosimetry from the two signals employed involves evaluation of the ratio of the Power spectrum of the thermal noise voltage that is Power SNR, to that of the induced voltage at the EMF-sensitive target pathway structure. The target pathway structure used is ion binding at receptor sites on Jurkat cells suspended in 2 mm of culture medium. The average peak electric field at the binding site from a PEMF signal comprising 5 msec burst of 200 μsec pulses repeating at 15 /sec, was 1 mV/cm, while for a 60 Hz signal it was 50 μV/cm.

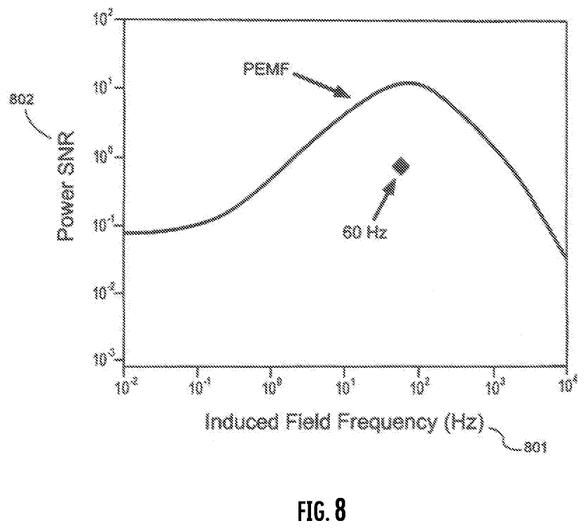

FIG. 8 is a graph of results wherein Induced Field Frequency 801 in Hz is plotted on the x-axis and Power SNR 802 is plotted on the y-axis. FIG. 8 illustrates that both signals have sufficient Power spectrum that is Power SNR 1, to be detected within a frequency range of binding kinetics. However, maximum Power SNR for the PEMF signal is significantly higher than that for the 60 Hz signal. This is because a PEMF signal has many frequency components falling within the bandpass of the binding pathway. The single frequency component of a 60 Hz signal lies at the mid-point of the bandpass of the target pathway. The Power SNR calculation that was used in this example is dependant upon T.sub.ion which is obtained from the rate constant for ion binding. Had this calculation been performed a priori it would have concluded that both signals satisfied basic detectability requirements and could modulate an EMF-sensitive ion binding pathway at the start of a regulatory cascade for DNA synthesis in these cells. The previous examples illustrated that utilizing the rate constant for Ca/CaM binding could lead to successful projections for bioeffective EMF signals in a variety of systems.

Induced time-varying currents from PEMF or PRF devices flow in a target pathway structure such as a molecule, cell, tissue, and organ, and it is these currents that are a stimulus to which cells and tissues can react in a physiologically meaningful manner The electrical properties of a target pathway structure affect levels and distributions of induced current. Molecules, cells, tissue, and organs are all in an induced current pathway such as cells in a gap junction contact. Ion or ligand interactions at binding sites on macromolecules that may reside on a membrane surface are voltage dependent processes, that is electrochemical, that can respond to an induced electromagnetic field (“E”). Induced current arrives at these sites via a surrounding ionic medium. The presence of cells in a current pathway causes an induced current (“J”) to decay more rapidly with time (“J(t)”). This is due to an added electrical impedance of cells from membrane capacitance and time constants of binding and other voltage sensitive membrane processes such as membrane transport.

Equivalent electrical circuit models representing various membrane and charged interface configurations have been derived. For example, in Calcium (“Ca.sup.2+”) binding, the change in concentration of bound Ca.sup.2+ at a binding site due to induced E may be described in a frequency domain by an impedance expression such as:

Z_{b}(ω)=Rᵢₒₙ+1/iωCᵢₒₙ

which has the form of a series resistance-capacitance electrical equivalent circuit. Where ω is angular frequency defined as 2πf, where f is frequency, i=−1^{1/2}, Z_{b}(ω) is the binding impedance, and Rᵢₒₙ and Cᵢₒₙ are equivalent binding resistance and capacitance of an ion binding pathway. The value of the equivalent binding time constant, τᵢₒₙ=RᵢₒₙCᵢₒₙ, is related to an ion binding rate constant, k_{b}, via τᵢₒₙ=RᵢₒₙCᵢₒₙ=1/k_{b}. Thus, the characteristic time constant of this pathway is determined by ion binding kinetics.

Induced E from a PEMF or PRF signal can cause current to flow into an ion binding pathway and affect the number of Ca.sup.2+ ions bound per unit time. An electrical equivalent of this is a change in voltage across the equivalent binding capacitance C.sub.ion, which is a direct measure of the change in electrical charge stored by C.sub.ion. Electrical charge is directly proportional to a surface concentration of Ca.sup.2+ ions in the binding site, that is storage of charge is equivalent to storage of ions or other charged species on cell surfaces and junctions. Electrical impedance measurements, as well as direct kinetic analyses of binding rate constants, provide values for time constants necessary for configuration of a PMF waveform to match a bandpass of target pathway structures. This allows for a required range of frequencies for any given induced E waveform for optimal coupling to target impedance, such as bandpass.

Ion binding to regulatory molecules is a frequent EMF target, for example Ca.sup.2+ binding to calmodulin (“CaM”). Use of this pathway is based upon acceleration of wound repair, for example bone repair, that involves modulation of growth factors released in various stages of repair. Growth factors such as platelet derived growth factor (“PDGF”), fibroblast growth factor (“FGF”), and epidermal growth factor (“EGF”) are all involved at an appropriate stage of healing. Angiogenesis is also integral to wound repair and modulated by PMF. All of these factors are Ca/CaM-dependent.

Utilizing a Ca/CaM pathway a waveform can be configured for which induced power is sufficiently above background thermal noise power. Under correct physiological conditions, this waveform can have a physiologically significant bioeffect.

Application of a Power SNR model to Ca/CaM requires knowledge of electrical equivalents of Ca²+ binding kinetics at CaM. Within first order binding kinetics, changes in concentration of bound Ca²+ at CaM binding sites over time may be characterized in a frequency domain by an equivalent binding time constant, τᵢₒₙ=RᵢₒₙCᵢₒₙ, where Rᵢₒₙ and Cᵢₒₙ are equivalent binding resistance and capacitance of the ion binding pathway. τ.sub.ion is related to a ion binding rate constant, k_{b}, via τ.ₒₙ=1/k_{b}. Published values for k_{b} can then be employed in a cell array model to evaluate SNR by comparing voltage induced by a PRF signal to thermal fluctuations in voltage at a CaM binding site. Employing numerical values for PMF response, such as Vₘₐₓ=6.5×10⁻⁷ sec⁻¹,=2.5 μM, KD=30 μM, [Ca2+CaM]=KD(+[CaM]), yields k_{b}=665 sec⁻¹=1.5 msec). Such a value for τᵢₒₙ can be employed in an electrical equivalent circuit for ion binding while power SNR analysis can be performed for any waveform structure.

According to an embodiment of the present invention a mathematical model can be configured to assimilate that thermal noise is present in all voltage dependent processes and represents a minimum threshold requirement to establish adequate SNR. Power spectral density, Sn(ω), of thermal noise can be expressed as:

Sₙ(ω)=4kTRe[Z_{M}(x,ω)]

where Z_{M}(x, ω) is electrical impedance of a target pathway structure, x is a dimension of a target pathway structure and Re denotes a real part of impedance of a target pathway structure. Z_{M}(x, ω) can be expressed as:

Z_{M} (x,ω)=[Re+Ri+Rg)/γ]tan h(γx)

This equation clearly shows that electrical impedance of the target pathway structure, and contributions from extracellular fluid resistance (“Rₑ”), intracellular fluid resistance (“Rᵢ”) and intermembrane resistance (“R_{g}”) which are electrically connected to a target pathway structure, all contribute to noise filtering.

A typical approach to evaluation of SNR uses a single value of a root mean square (RMS) noise voltage. This is calculated by taking a square root of an integration of

Sₙ(ω)=4kTRe[Z_{M}(xω)]

over all frequencies relevant to either complete membrane response, or to bandwidth of a target pathway structure. SNR can be expressed by a ratio:

SNR =|VM(ω)|/RMS

where |(V.sub.M(ω))| is maximum amplitude of voltage at each frequency as delivered by a chosen waveform to the target pathway structure.

An embodiment according to the present invention comprises a pulse burst envelope having a high spectral density, so that the effect of therapy upon the relevant dielectric pathways, such as, cellular membrane receptors, ion binding to cellular enzymes and general transmembrane potential changes, is enhanced. Accordingly, by increasing a number of frequency components transmitted to relevant cellular pathways, a large range of biophysical phenomena, such as modulating growth factor and cytokine release and ion binding at regulatory molecules, applicable to known healing mechanisms is accessible. According to an embodiment of the present invention applying a random, or other high spectral density envelope, to a pulse burst envelope of mono- or bi-polar rectangular or sinusoidal pulses inducing peak electric fields between about 10⁻⁶ and about 100 V/cm, produces a greater effect on biological healing processes applicable to both soft and hard tissues.

According to yet another embodiment of the present invention by applying a high spectral density voltage envelope as a modulating or pulse-burst defining parameter, power requirements for such amplitude modulated pulse bursts can be significantly lower than that of an unmodulated pulse burst containing pulses within a similar frequency range. This is due to a substantial reduction in duty cycle within repetitive burst trains brought about by imposition of an irregular, and preferably random, amplitude onto what would otherwise be a substantially uniform pulse burst envelope. Accordingly, the dual advantages, of enhanced transmitted dosimetry to the relevant dielectric pathways and of decreased power requirement are achieved.

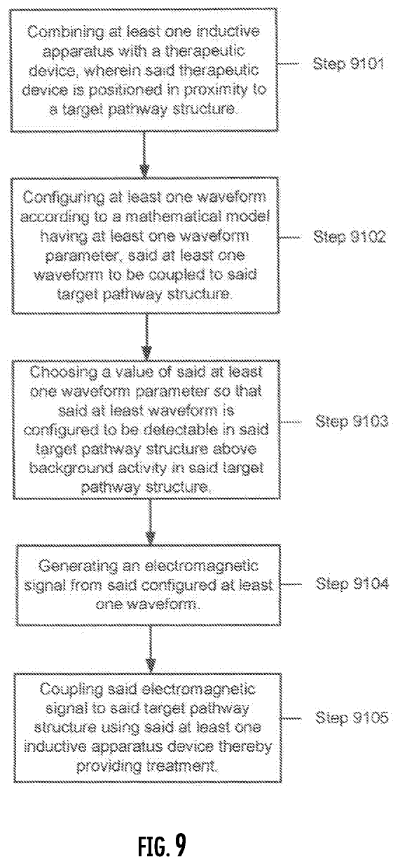

Referring to FIG. 9, wherein FIG. 9 is a flow diagram of a method of using an inductive apparatus to deliver electromagnetic signals to target pathway structures such as such as molecules, cells, tissues, and organs of plants, animals, and humans for therapeutic and prophylactic purposes according to an embodiment of the present invention. A lightweight inductive apparatus is integrated into at least one therapeutic device that will be used for treatment, however the inductive apparatus can also be attached to at least one therapeutic device (Step 9101). Miniaturized circuitry containing logic for a mathematical model having at least one waveform parameter used to configure at least one waveform to be coupled to a target pathway structure such as molecules, cells, tissues, and organs, is attached to the coil by at least one wire (Step 9102). However, the attachment can also be wireless. The configured waveform satisfies a SNR or Power SNR model so that for a given and known target pathway structure it is possible to choose at least one waveform parameter so that a waveform is detectable in the target pathway structure above its background activity (Step 9103) such as baseline thermal fluctuations in voltage and electrical impedance at a target pathway structure that depend upon a state of a cell and tissue, that is whether the state is at least one of resting, growing, replacing, and responding to injury. A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model (Step 9104). A repetitive electromagnetic signal can be generated for example inductively, from said configured at least one waveform (Step 9105). The repetitive electromagnetic signal can also be generated conductively. The electromagnetic signal is coupled to a target pathway structure such as molecules, cells, tissues, and organs by output of the inductive apparatus integrated into the support (Step 9106).

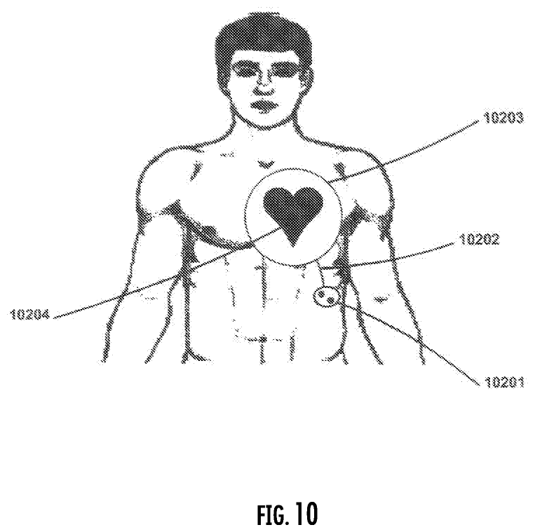

FIG. 10 illustrates a preferred embodiment of an apparatus according to the present invention. A miniature control circuit 10201, which may be similar or identical to the control circuit 300 shown in FIG. 3, is coupled to an end of at least one connector 10202 such as wire. The opposite end of the at least one connector is coupled to a generating device such as a pair of electrical coils 10203. The generating device is constructed to have electrical properties that optimize generation of electromagnetic signals from waveforms configured to satisfy at least one of a SNR model, a Power SNR model, and any other mathematical model used for waveform configuration. The miniature control circuit 10201 is constructed in a manner that applies a mathematical model that is used to configure waveforms. The configured waveforms have to satisfy a SNR or Power SNR model so that for a given and known target pathway structure, it is possible to choose waveform parameters that satisfy SNR or Power SNR so that a waveform is detectable in the target pathway structure above its background activity. A preferred embodiment according to the present invention applies a mathematical model to induce a time-varying magnetic field and a time-varying electric field in a target pathway structure such as molecules, cells, tissues, and organs, comprising about 10 to about 100 msec bursts of about 1 to about 100 microsecond rectangular pulses repeating at about 0.1 to about 10 pulses per second. Peak amplitude of the induced electric field is between about 1 μV/cm and about 100 mV/cm, varied according to a modified 1/f function where f=frequency. A waveform configured using a preferred embodiment according to the present invention may be applied to a target pathway structure such as molecules, cells, tissues, and organs for a preferred total exposure time of under 1 minute to 240 minutes daily. However other exposure times can be used. Waveforms configured by the miniature control circuit 10201 are directed to a generating device 10203 such as electrical coils via connector 10202. The generating device 10203 delivers a pulsing magnetic field configured according to a mathematical model, that can be used to provide treatment to a target pathway structure such as a heart in a chest 10204. The miniature control circuit applies a pulsing magnetic field for a prescribed time and can automatically repeat applying the pulsing magnetic field for as many applications as are needed in a given time period, for example 10 times a day. A preferred embodiment according to the present invention can be positioned to treat the heart in a chest 10204 by a positioning device. Coupling a pulsing magnetic field to a angiogenesis and neovascularization target pathway structure such as ions and ligands, therapeutically and prophylactically reduces inflammation thereby reducing pain and promotes healing. When electrical coils are used as the generating device 10203, the electrical coils can be powered with a time varying magnetic field that induces a time varying electric field in a target pathway structure according to Faraday's law. An electromagnetic signal generated by the generating device 10203 can also be applied using electrochemical coupling, wherein electrodes are in direct contact with skin or another outer electrically conductive boundary of a target pathway structure. In yet another embodiment according to the present invention, the electromagnetic signal generated by the generating device 10203 can also be applied using electrostatic coupling wherein an air gap exists between a generating device 10203 such as an electrode and a target pathway structure such as molecules, cells, tissues, and organs. An advantage of the preferred embodiment according to the present invention is that its ultra-lightweight coils and miniaturized circuitry allow for use with common physical therapy treatment modalities and at any body location for which pain relief and healing is desired. An advantageous result of application of the preferred embodiment according to the present invention is that a living organism's angiogenesis and neovascularization can be maintained and enhanced.

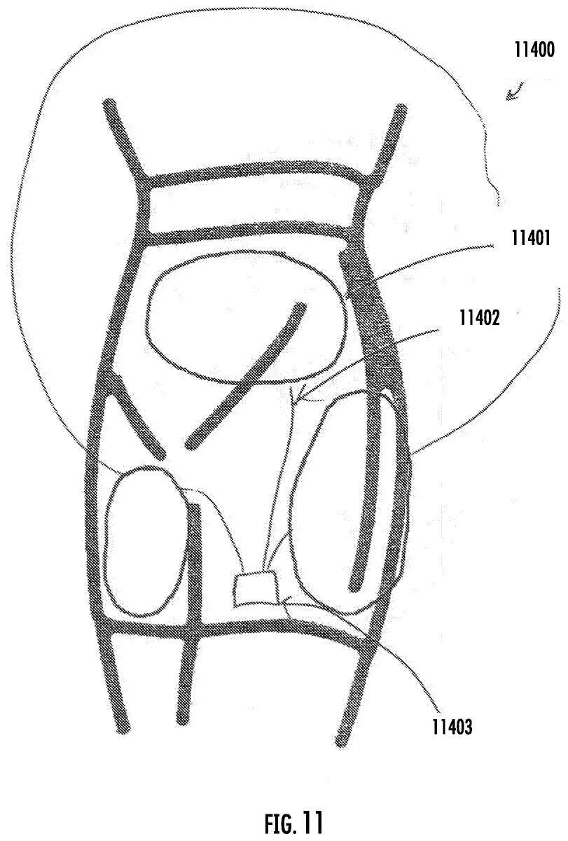

Referring to FIG. 11, an embodiment according to the present invention of an electromagnetic treatment inductive apparatus integrated into hip, thigh, and lower back support garment 12400 is illustrated. Several lightweight flexible coils 12401 are integrated into the support garment. The lightweight flexible coils can be constructed from fine flexible conductive wire, conductive thread, and any other flexible conductive material. The flexible coils are connected to at least one end of at least one wire 12402. However, the flexible coils can also be configured to be directly connected to circuitry 12403 or wireless. Lightweight miniaturized circuitry 12403 that configures waveforms according to an embodiment of the present invention, is attached to at least one other end of said at least on wire. When activated the lightweight miniaturized circuitry 12403 configures waveforms that are directed to the flexible coils (12401) to create PEMF signals that are coupled to a target pathway structure.

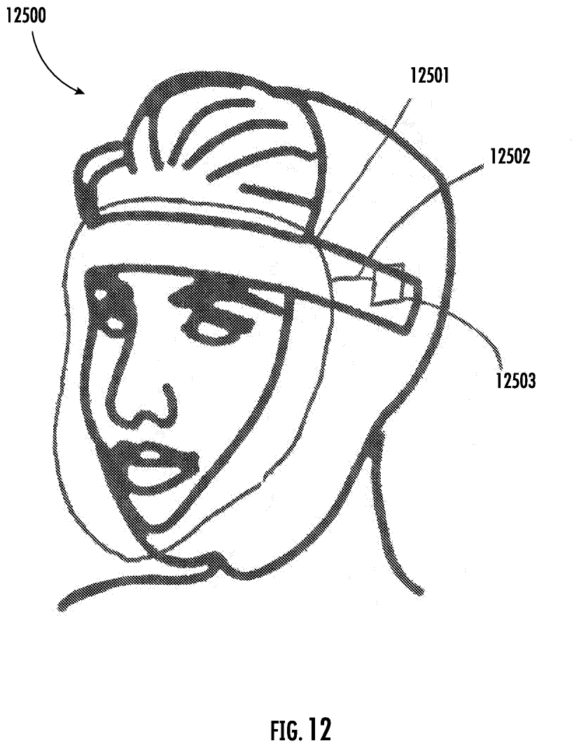

Referring to FIG. 12, an embodiment according to the present invention of an electromagnetic treatment inductive apparatus integrated into a head and face support garment 12500 is illustrated. Several lightweight flexible coils 12501 are integrated into the support garment. The lightweight flexible coils can be constructed from fine flexible conductive wire, conductive thread, and any other flexible conductive material. The flexible coils are connected to at least one end of at least one wire 12502. However, the flexible coils can also be configured to be directly connected to circuitry 12503 or wireless. Lightweight miniaturized circuitry 12503 that configures waveforms, according to an embodiment of the present invention, is attached to at least one other end of said at least on wire. When activated the lightweight miniaturized circuitry 12503 configures waveforms that are directed to the flexible coils 12350) to create PEMF signals that are coupled to a target pathway structure.

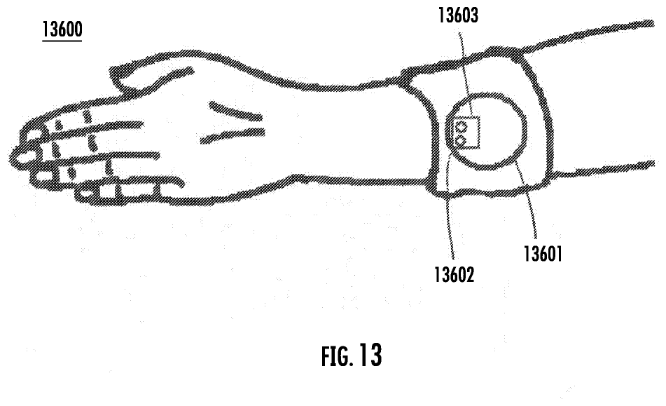

Referring to FIG. 13, an embodiment according to the present invention of an electromagnetic treatment inductive apparatus integrated into surgical dressing applied to a human forearm 14600 is illustrated. Several lightweight flexible coils 14601 are integrated into the dressing. The lightweight flexible coils can be constructed from fine flexible conductive wire, conductive thread, and any other flexible conductive material. The flexible coils are connected to at least one end of at least one wire 14602. However, the flexible coils can also be configured to be directly connected to circuitry 14603 or wireless. Lightweight miniaturized circuitry 14603 that configures waveforms according to an embodiment of the present invention, is attached to at least one other end of said at least one wire. When activated the lightweight miniaturized circuitry 14603 configures waveforms that are directed to the flexible coils (14601) to create PEMF signals that are coupled to a target pathway structure.

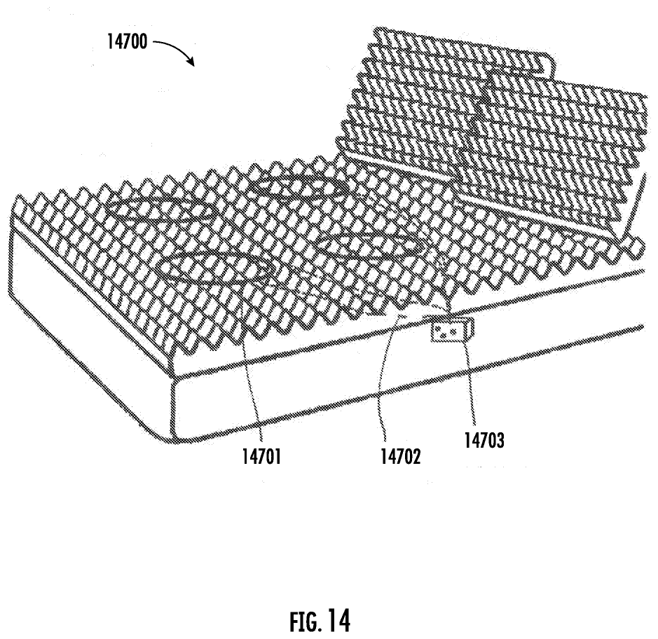

Referring to FIG. 14, an embodiment according to the present invention of an electromagnetic treatment inductive apparatus integrated into a mattress pad 14700 is illustrated. Several lightweight flexible coils 14701 are integrated into the mattress pad. The lightweight flexible coils can be constructed from fine flexible conductive wire, conductive thread, and any other flexible conductive material. The flexible coils are connected to at least one end of at least one wire 14702. However, the flexible coils can also be configured to be directly connected to circuitry 14703 or wireless. Lightweight miniaturized circuitry 15403 that configures waveforms according to an embodiment of the present invention, is attached to at least one other end of said at least on wire. When activated the lightweight miniaturized circuitry 14703 configures waveforms that are directed to the flexible coils (14701) to create PEMF signals that are coupled to a target pathway structure.

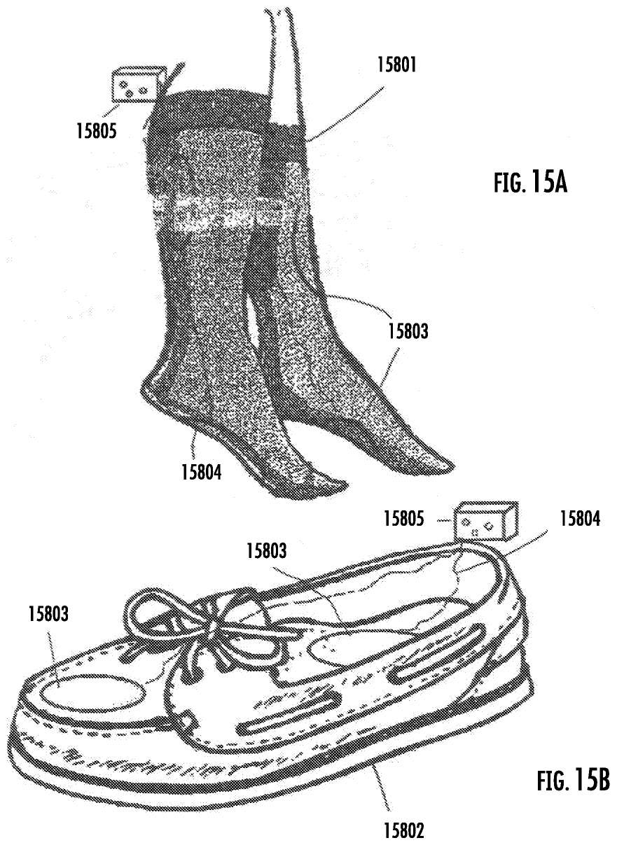

Referring to FIGS. 15A and 15B, an embodiment according to the present invention of an electromagnetic treatment inductive apparatus integrated into a sock 15801 and a shoe 15802 are illustrated. Several lightweight flexible coils 15803 are integrated into the dressing. The lightweight flexible coils can be constructed from fine flexible conductive wire, conductive thread, and any other flexible conductive material. The flexible coils are connected to at least one end of at least one wire 15804. However, the flexible coils can also be configured to be directly connected to circuitry 15805 or wireless. Lightweight miniaturized circuitry 15805 that configures waveforms according to an embodiment of the present invention, is attached to at least one other end of said at least on wire. When activated the lightweight miniaturized circuitry 15805 configures waveforms that are directed to the flexible coils 15806 to create PEMF signals that are coupled to a target pathway structure.

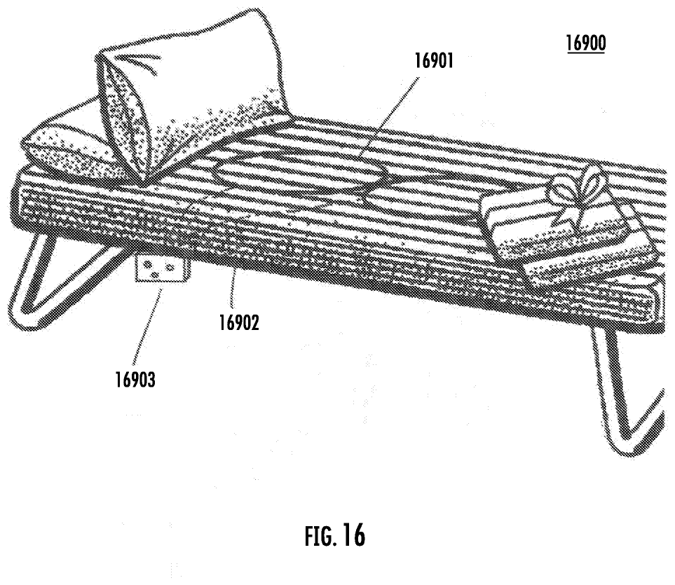

Referring to FIG. 16, an embodiment according to the present invention of an electromagnetic treatment inductive apparatus integrated into a therapeutic bed 16900 is illustrated. Several lightweight flexible coils 16901 are integrated into the bed. The lightweight flexible coils can be constructed from fine flexible conductive wire, conductive thread, and any other flexible conductive material. The flexible coils are connected to at least one end of at least one wire 16902. However, the flexible coils can also be configured to be directly connected to circuitry 16903 or wireless. Lightweight miniaturized circuitry 16903 that configures waveforms according to an embodiment of the present invention, is attached to at least one other end of said at least on wire. When activated the lightweight miniaturized circuitry 16903 configures waveforms that are directed to the flexible coils 16901 to create PEMF signals that are coupled to a target pathway structure.

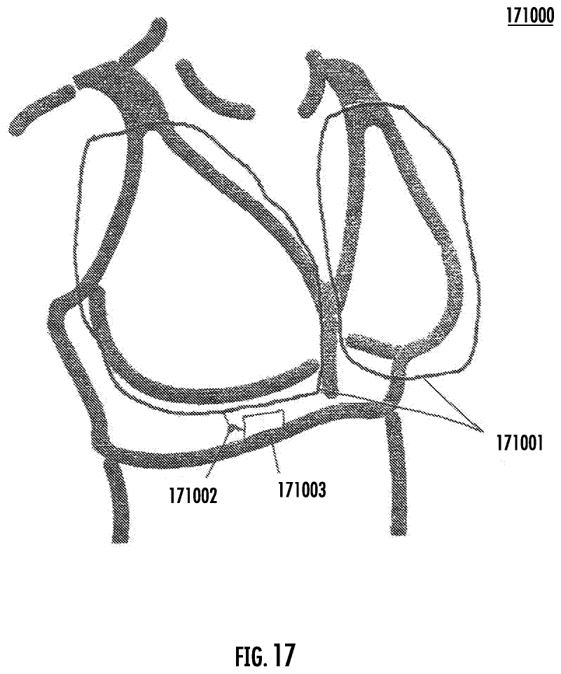

Referring to FIG. 17, an embodiment according to the present invention of an electromagnetic treatment inductive apparatus integrated into a chest garment 171000, such as a bra is illustrated. Several lightweight flexible coils 171001 are integrated into a bra. The lightweight flexible coils can be constructed from fine flexible conductive wire, conductive thread, and any other flexible conductive material. The flexible coils are connected to at least one end of at least one wire 171002. However, the flexible coils can also be configured to be directly connected to circuitry 171003 or wireless. Lightweight miniaturized circuitry 171003 that configures waveforms according to an embodiment of the present invention, is attached to at least one other end of said at least on wire. When activated the lightweight miniaturized circuitry 171003 configures waveforms that are directed to the flexible coils 171001 to create PEMF signals that are coupled to a target pathway structure.

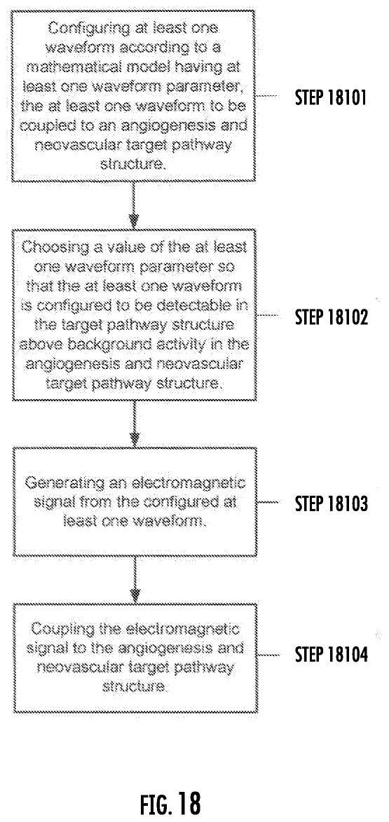

Referring to FIG. 18, wherein FIG. 18 is a flow diagram of a method for delivering electromagnetic signals to angiogenesis and neovascularization target pathway structures such as ions and ligands of plants, animals, and humans for therapeutic and prophylactic purposes according to an embodiment of the present invention. A mathematical model having at least one waveform parameter is applied to configure at least one waveform to be coupled to a angiogenesis and neovascularization target pathway structure such as ions and ligands (Step 18101). The configured waveform satisfies a SNR or Power SNR model so that for a given and known angiogenesis and neovascularization target pathway structure it is possible to choose at least one waveform parameter so that a waveform is detectable in the angiogenesis and neovascularization target pathway structure above its background activity (Step 18102) such as baseline thermal fluctuations in voltage and electrical impedance at a target pathway structure that depend upon a state of a cell and tissue, that is whether the state is at least one of resting, growing, replacing, and responding to injury. A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model (Step 18102). A repetitive electromagnetic signal can be generated for example inductively or capacitively, from said configured at least one waveform (Step 18103). The electromagnetic signal is coupled to an angiogenesis and neovascularization target pathway structure such as ions and ligands by output of a coupling device such as an electrode or an inductor, placed in close proximity to the target pathway structure (Step 18104). The coupling enhances modulation of binding of ions and ligands to regulatory molecule in living tissues and cells.

### EXAMPLE 4

In this example electromagnetic field energy was used to stimulate neovascularization in an in vivo model. Two different signal were employed, one configured according to prior art and a second configured according to an embodiment of the present invention.

One hundred and eight Sprague-Dawley male rats weighing approximately 300 grams each, were equally divided into nine groups. All animals were anesthetized with a mixture of ketamine/acepromazine/Stadol at 0.1 cc/g. Using sterile surgical techniques, each animal had a 12 cm to 14 cm segment of tail artery harvested using microsurgical technique. The artery was flushed with 60 U/ml of heparinized saline to remove any blood or emboli.

These tail vessels, with an average diameter of 0.4 mm to 0.5 mm, were then sutured to the transected proximal and distal segments of the right femoral artery using two end-to-end anastomoses, creating a femoral arterial loop. The resulting loop was then placed in a subcutaneous pocket created over the animal's abdominal wall/groin musculature, and the groin incision was closed with 4-0 Ethilon. Each animal was then randomly placed into one of nine groups: groups 1 to 3 (controls), these rats received no electromagnetic field treatments and were killed at 4, 8, and 12 weeks; groups 4 to 6, 30 min. treatments twice a day using 0.1 gauss electromagnetic fields for 4, 8, and 12 weeks (animals were killed at 4, 8, and 12 weeks, respectively); and groups 7 to 9, 30 min. treatments twice a day using 2.0 gauss electromagnetic fields for 4, 8, and 12 weeks (animals were killed at 4, 8, and 12 weeks, respectively).

Pulsed electromagnetic energy was applied to the treated groups using a device constructed according to an embodiment of the present invention Animals in the experimental groups were treated for 30 minutes twice a day at either 0.1 gauss or 2.0 gauss, using short pulses (2 msec to 20 msec) 27.12 MHz. Animals were positioned on top of the applicator head and confined to ensure that treatment was properly applied. The rats were reanesthetized with ketamine/acepromazine/Stadol intraperitoneally and 100 U/kg of heparin intravenously. Using the previous groin incision, the femoral artery was identified and checked for patency. The femoral/tail artery loop was then isolated proximally and distally from the anastomoses sites, and the vessel was clamped off. Animals were then killed. The loop was injected with saline followed by 0.5 cc to 1.0 cc of colored latex through a 25-gauge cannula and clamped. The overlying abdominal skin was carefully resected, and the arterial loop was exposed. Neovascularization was quantified by measuring the surface area covered by new blood-vessel formation delineated by the intraluminal latex. All results were analyzed using the SPSS statistical analysis package.

The most noticeable difference in neovascularization between treated versus untreated rats occurred at week 4. At that time, no new vessel formation was found among controls, however, each of the treated groups had similar statistically significant evidence of neovascularization at 0 cm2 versus 1.42±0.80 cm2 (p<0.001). These areas appeared as a latex blush segmentally distributed along the sides of the arterial loop. At 8 weeks, controls began to demonstrate neovascularization measured at 0.7±0.82 cm². Both treated groups at 8 weeks again had approximately equal statistically significant (p<0.001) outcroppings of blood vessels of 3.57±1.82 cm2 for the 0.1 gauss group and of 3.77±1.82 cm² for the 2.0 gauss group. At 12 weeks, animals in the control group displayed 1.75±.0.95 cm2 of neovascularization, whereas the 0.1 gauss group demonstrated 5.95±3.25 cm², and the 2.0 gauss group showed 6.20±3.95 cm² of arborizing vessels. Again, both treated groups displayed comparable statistically significant findings (p<0.001) over controls.

These experimental findings demonstrate that electromagnetic field stimulation of an isolated arterial loop according to an embodiment of the present invention increases the amount of quantifiable neovascularization in an in vivo rat model. Increased angiogenesis was demonstrated in each of the treated groups at each of the sacrifice dates. No differences were found between the results of the two gauss levels tested as predicted by the teachings of the present invention.

An embodiment according to the present invention provides a higher spectral density to a pulse burst envelope resulting in enhanced effectiveness of therapy upon relevant dielectric pathways, such as, cellular membrane receptors, ion binding to cellular enzymes and general transmembrane potential changes. An embodiment according to the present invention increases the number of frequency components transmitted to relevant cellular pathways, thereby providing access to a larger range of biophysical phenomena applicable to known healing mechanisms, for example modulation of growth factor and cytokine release, and ion binding at regulatory molecules. By applying a random, or other high spectral density envelope, according to a mathematical model defined by SNR or Power SNR in a transduction pathway, to a pulse burst envelope of mono- or bi-polar rectangular or sinusoidal pulses inducing peak electric fields between 10⁻⁶ and 10 volts per centimeter (V/cm), a greater effect could be accomplished on biological healing processes applicable to both soft and hard tissues.

An advantageous result of the present invention, is that by applying a high spectral density voltage envelope as the modulating or pulse-burst defining parameter, according to a mathematical model defined by SNR or Power SNR in a transduction pathway, the power requirement for such amplitude modulated pulse bursts can be significantly lower than that of an unmodulated pulse burst containing pulses within the same frequency range. Accordingly, the advantages of enhanced transmitted dosimetry to the relevant dielectric target pathways and of decreased power requirement are achieved. Another advantage of the present invention is the acceleration of wound repair.

Known mechanisms of wound repair involve the naturally timed release of the appropriate growth factor or cytokine in each stage of wound repair as applied to humans, animals and plants. Specifically, wound repair involves an inflammatory phase, angiogenesis, cell proliferation, collagen production, and remodeling stages. There are timed releases of specific cytokines and growth factors in each stage. Electromagnetic fields are known to enhance blood flow and to enhance the binding of ions which, in turn, can accelerate each healing phase. It is an object of this invention to provide an improved means to enhance the action and accelerate the intended effects or improve efficacy as well as other effects of the cytokines and growth factors relevant to each stage of wound repair.

An embodiment according to the present invention comprises a pulse burst envelope having a high spectral density, so that the effect of therapy upon the relevant dielectric pathways, such as, cellular membrane receptors, ion binding to cellular enzymes and general transmembrane potential changes, is enhanced. Accordingly, by increasing a number of frequency components transmitted to relevant cellular pathways, a large range of biophysical phenomena, such as modulating growth factor and cytokine release and ion binding at regulatory molecules, applicable to known tissue growth mechanisms is accessible. According to an embodiment of the present invention applying a random, or other high spectral density envelope, to a pulse burst envelope of mono- or bi-polar rectangular or sinusoidal pulses inducing peak electric fields between about 10⁻⁸ and about 100 V/cm, produces a greater effect on biological healing processes applicable to both soft and hard tissues.

According to yet another embodiment of the present invention by applying a high spectral density voltage envelope as a modulating or pulse-burst defining parameter, power requirements for such amplitude modulated pulse bursts can be significantly lower than that of an unmodulated pulse burst containing pulses within a similar frequency range. This is due to a substantial reduction in duty cycle within repetitive burst trains brought about by imposition of an irregular, and preferably random, amplitude onto what would otherwise be a substantially uniform pulse burst envelope. Accordingly, the dual advantages, of enhanced transmitted dosimetry to the relevant dielectric pathways and of decreased power requirement are achieved.

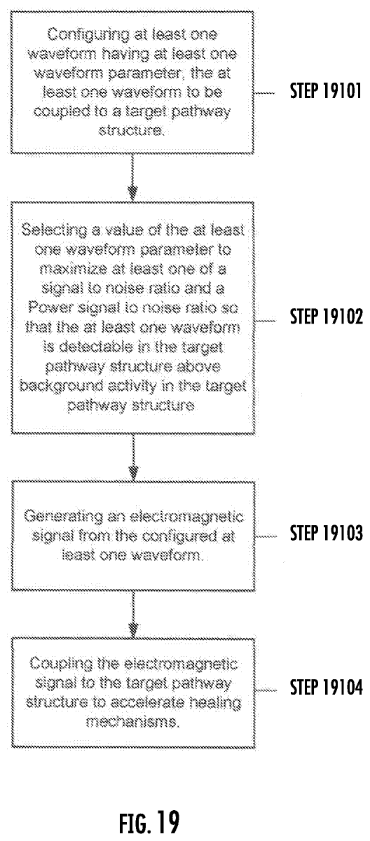

Referring to FIG. 19, wherein FIG. 19 is a flow diagram of a method according to an embodiment of the present invention, for accelerating wound repair by delivering electromagnetic signals that can be pulsed, to target pathway structures such as ions and ligands of animals and humans, for therapeutic and prophylactic purposes. Target pathway structures can also include but are not limited to tissues, cells, organs, and molecules.

Configuring at least one waveform having at least one waveform parameter to be coupled to the target pathway structure such as ions and ligands (Step 23101).

The at least one waveform parameter is selected to maximize at least one of a signal to noise ratio and a Power Signal to Noise ratio in a target pathway structure so that a waveform is detectable in the target pathway structure above its background activity (Step 23102) such as baseline thermal fluctuations in voltage and electrical impedance at a target pathway structure that depend upon a state of a cell and tissue, that is whether the state is at least one of resting, growing, replacing, and responding to injury to produce physiologically beneficial results. To be detectable in the target pathway structure the value of said at least one waveform parameter is chosen by using a constant of said target pathway structure to evaluate at least one of a signal to noise ratio, and a Power signal to noise ratio, to compare voltage induced by said at least one waveform in said target pathway structure to baseline thermal fluctuations in voltage and electrical impedance in said target pathway structure whereby bioeffective modulation occurs in said target pathway structure by said at least one waveform by maximizing said at least one of signal to noise ratio and Power signal to noise ratio, within a bandpass of said target pathway structure.

A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model (Step 23103). A repetitive electromagnetic signal can be generated for example inductively or capacitively, from said configured at least one waveform (Step 23104). The electromagnetic signal can also be non-repetitive. The electromagnetic signal is coupled to a target pathway structure such as ions and ligands by output of a coupling device such as an electrode or an inductor, placed in close proximity to the target pathway structure (Step 23105). The coupling enhances blood flow and modulation of binding of ions and ligands to regulatory molecules in molecules, tissues, cells, and organs thereby accelerating wound repair.

In one embodiment, the method shown in FIG. 19 may be performed using a self-contained, lightweight, and portable apparatus such as that shown in FIG. 2. The configured waveforms have to satisfy Power SNR so that for a given and known target pathway structure, it is possible to choose waveform parameters that satisfy Power SNR so that a waveform produces physiologically beneficial results, for example bioeffective modulation, and is detectable in the target pathway structure above its background activity.

In a preferred embodiment of the method of FIG. 19 according to the present invention, a mathematical model is applied to induce a time-varying magnetic field and a time-varying electric field in a target pathway structure such as ions and ligands, comprising about 0.1 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency. In a preferred embodiment, a waveform configured having the same parameters discussed above in connection with FIG. 5 may be applied to a target pathway structure such as ions and ligands for a preferred total exposure time of under 1 minute to 240 minutes daily. However other exposure times can be used. Waveforms configured by the miniature control circuit 201 which may be similar or identical to the control circuit 300 shown in FIG. 3, are directed to a generating device 203 such as electrical coils via connector 202. The generating device 203 delivers a pulsing magnetic field that can be used to provide treatment to a target pathway structure such as tissue. The miniature control circuit applies a pulsing magnetic field for a prescribed time and can automatically repeat applying the pulsing magnetic field for as many applications as are needed in a given time period, for example 10 times a day. The miniature control circuit can be configured to be programmable applying pulsing magnetic fields for any time repetition sequence.

A preferred embodiment according to the present invention can accelerate wound repair by being incorporated into a positioning device 204. Coupling a pulsing magnetic field to a target pathway structure such as ions and ligands, therapeutically and prophylactically reduces inflammation thereby advantageously reducing pain, promoting healing in targeted areas. When electrical coils are used as the generating device 203, the electrical coils can be powered with a time varying magnetic field that induces a time varying electric field in a target pathway structure according to Faraday's law. An electromagnetic signal generated by the generating device 203 can also be applied using electrochemical coupling, wherein electrodes are in direct contact with skin or another outer electrically conductive boundary of a target pathway structure. In yet another embodiment according to the present invention, the electromagnetic signal generated by the generating device 203 can also be applied using electrostatic coupling wherein an air gap exists between a generating device 203 such as an electrode and a target pathway structure such as ions and ligands. An advantage of the preferred embodiment according to the present invention is that its ultra-lightweight coils and miniaturized circuitry allow for use with common physical therapy treatment modalities and at any for which growth, pain relief, and tissue and organ healing is desired. An advantageous result of application of the preferred is that growth, repair, and maintenance of molecules, cells, tissues, and organs can be accomplished and enhanced anywhere and at any time, for example while driving a car or watching television.

In the embodiment associated with FIG. 19, the electromagnetic signal may be coupled to a target pathway structure by a coupling device having the configuration shown in FIGS. 4A and B. In this embodiment, the coupling device 400 can be an electric coil 401 wound with single or multistrand flexible wire 402; however solid wire can also be used. In a preferred embodiment according to the present invention, the wire is made of copper but other materials can be used. The multistrand flexible magnetic wire 402 enables the electric coil 401 to conform to specific anatomical configurations such as a limb or joint of a human or animal. In an apparatus for wound treatment, the electric coil 401 may comprise about 1 to about 1000 turns of about 0.01 mm to about 0.1 mm diameter at least one of single magnet wire and multistrand magnet wire, wound on an initially circular form having an outer diameter between about 2.5 cm and about 50 cm but other numbers of turns and wire diameters can be used. The electric coil 26401 can be encased with a non-toxic PVC mould 403 but other non-toxic moulds can also be used. The electric coil can also be incorporated in dressings, bandages, garments, and other structures typically used for wound treatment.

In other embodiments, an apparatus for performing the method of FIG. 19 may also incorporated into a positioning device such as the wrist support 600 shown in FIG. 6, or other devices such as knee, elbow, lower back, shoulder, other anatomical wraps, and apparel such as garments, fashion accessories, and footwear. Alternatively, the apparatus may be incorporated into the mattress pad of FIG. 14, or into a chest garment such as the bra of FIG. 17.

### EXAMPLE 5

This study demonstrated the effect of electromagnetic fields configured according an embodiment of the present invention accelerate tendon repair in an in-vivo model.

Young adult male Sprague-Dawley rats, with a mean weight of 350 g, were anesthetized with an intraperitoneal injection of a ketamine/medetomidine 75 mg/kg/0.5 mg/kg mixture. The Achilles tendon was disrupted and repaired. Using sterile surgical technique, a 2-cm midline longitudinal incision was made over the right Achilles tendon while it was stretched by flexing the right foot. Blunt dissection was used to separate the tendon from the surrounding tissue, which was then transected at the middle using a scalpel. The Achilles tendon was then immediately repaired with 6-0 nylon suture using a modified Kessler stitch. The plantaris tendon was divided and not repaired. The skin was sutured over the repaired tendon using interrupted 5-0 Ethilon. The Achilles tendon was not immobilized. Postoperatively, the animals received Ketoprofen for pain control.

On the first postoperative day, all animals were randomly assigned to four treatment groups with 10 animals in each group. Randomization followed the parallel group protocol wherein each animal was randomly assigned to one treatment group until there were ten in each group. Animals remained in their assigned group. There were three active groups that received specific EMF treatments for two 30-min sessions per day over a period of 3 weeks, and one identically treated sham group. The EMF employed in this study was a pulsed radio frequency waveform comprising a repetitive burst of 27.12 MHz sinusoidal waves emitted by a PMF-generating coil. Two configurations were employed. The first, assigned to Group 1, comprised a burst duration of 65 μsec, repeating at 600 bursts/sec with an amplitude at the tendon target of 1 gauss (“G”). The second PRF waveform comprised a burst duration of 2000 μsec, repeating at 5 bursts/sec with an amplitude at the tendon target of 0.05 G, assigned to Group 2, and 0.1 G, assigned to Group 3. Sham animals, no signal, were assigned to Group 4.

The PRF signal was delivered with a single loop coil, mounted to enable a standard rat plastic cage, with all metal portions removed, to be positioned within it. The coil was located 3.5 inches above, and horizontal to, the floor of the cage. Five freely roaming animals were treated with each coil. EMF signal amplitude was checked. Signal amplitude within the rat treatment cage over the normal range of rat movement was uniform to ±10%. Signal consistency was verified weekly. There were two cages each for the sham and active groups, and each cage had its individual coded EMF exposure system. EMF treatment was carried out twice daily for 30-min sessions until sacrifice. Sham animals were treated in identical cages equipped with identical coils.

At the end of the 3-week treatment period, the Achilles tendon was harvested by proximally severing the muscle bellies arising from the tendon and distally disarticulating the ankle, keeping the calcaneous and foot attached. All extraneous soft and hard tissues were removed from the calcaneous-Achilles tendon complex. Tensile strength testing was done immediately after harvest. The tendon, in continuity with the calcaneal bone, was fixed between two metal clamps so as to maintain a physiologically appropriate foot dorsiflexion, compared to the vertically oriented Achilles tendon. The tendons were then pulled apart at a constant speed of 0.45 mm/sec until failure, and the peak tensile strength was recorded. All analyzable tendons failed at the original transection. The tensile strengths from a total of 38 tendons were available for analysis.

Mean tensile strength was compared for each group at 3 weeks post tendon transection and data were analyzed. Tensile strength was calculated as the maximum breaking strength in kilograms per cross-sectional area in square centimeters. Tendons treated with the 65 μsec signal in Group 1 had a mean breaking strength of 99.4±14.6 kg/cm² compared to 80.6±16.6 kg/cm² for the sham-treated group in Group 4. This represented a 24% increase in breaking strength vs. the sham group at 21 days, which was not statistically significant (p=0.055). Tendons from Groups 2 and 3, treated with the 2000 μsec signals, had significantly higher mean breaking strengths of 129.4±27.8 kg/cm² and 136.4±31.6 kg/cm² for the 0.05 G and 0.1 G signals, respectively, vs. the sham exposure group 80.6±16.6 kg/cm². The mean strengths for both Groups 2 and 3 were 60% and 69% higher, respectively, at the end of 3 weeks of treatment, compared to the sham group. This increase in strength was statistically significant (p<0.001); however, the difference in mean tensile strength between Groups 2 and 3 was not statistically significant (p=0.541). The differences in mean tensile strength between Group 1 (65 μsec burst) and Groups 2 and 3 (2000 μsec burst) was statistically significant (p<0.05).

The results presented here demonstrate that non-invasive pulsed electromagnetic fields can produce up to a 69% increase in rat Achilles tendon breaking strength vs. sham-treated tendons at 21 days post transection. All signals utilized in this study accelerated tendon repair, however greatest acceleration was obtained with waveforms configured according to a transduction mechanism involving Ca²⁺ binding.

In a manner similar to bone and wound repair, tendon repair for both epitenon and synovial-sheathed tendons begins with an inflammatory stage that generally involves infiltration of inflammatory cells such as macrophages, neutrophils, and T-lymphocytes. This is followed by angiogenesis, fibroblast proliferation, and collagen mainly type III, production. Finally, cells and collagen fibrils orient to achieve maximum mechanical strength. These phases all occur in bone and wound repair, in which EMF has demonstrated effects, particularly in inflammatory, angiogenesis, and cell proliferation stages.

An EMF transduction pathway involves ion binding in regulatory pathways involving growth factor release. Production of many of the growth factors and cytokines involved in tissue growth and repair is dependent on Ca/CaM calmodulin. EMF has been shown to accelerate Ca²⁺ binding to calmodulin. The 0.05 and 0.1 G signals utilized in this study were configured using a Ca/CaM transduction pathway. The objective was to produce sufficient electric field amplitude that is dose, within the frequency response of Ca²⁺ binding. This would result in a lower power, more effective signal. The model demonstrated that microsecond range burst durations satisfy these objectives at amplitudes in the 0.05 G range. The 0.1 G signal was added to assure that the small size of the rat tendon target did not limit the induced current pathway and reduce the expected dose.

EMF accelerates bone repair by accelerating return to intact breaking strength. The sham-treated fractures eventually reach the same biomechanical end point, but with increased morbidity. Biomechanical acceleration in a linear full-thickness cutaneous wound in the rat was observed. EMF accelerated wound repair by approximately 60% at 21 days, with intact breaking strength achieved about 50% sooner than the untreated wounds.

An embodiment according to the present invention provides a higher spectral density to a pulse burst envelope resulting in enhanced effectiveness of therapy upon relevant dielectric pathways, such as, cellular membrane receptors, ion binding to cellular enzymes and general transmembrane potential changes. An embodiment according to the present invention increases the number of frequency components transmitted to relevant cellular pathways, thereby providing access to a larger range of biophysical phenomena applicable to known healing mechanisms, for example modulation of growth factor and cytokine release, and ion binding at regulatory molecules. By applying a random, or other high spectral density envelope, according to a mathematical model defined by SNR or Power SNR in a transduction pathway, to a pulse burst envelope of mono- or bi-polar rectangular or sinusoidal pulses inducing peak electric fields between 10⁻⁶ and 10 volts per centimeter (V/cm), a greater effect could be accomplished on biological healing processes applicable to both soft and hard tissues thereby enhancing effectiveness of pharmacological, chemical, cosmetic and topical agents.

An advantageous result of the present invention, is that by applying a high spectral density voltage envelope as the modulating or pulse-burst defining parameter, according to a mathematical model defined by SNR or Power SNR in a transduction pathway, the power requirement for such amplitude modulated pulse bursts can be significantly lower than that of an unmodulated pulse burst containing pulses within the same frequency range. Accordingly, the advantages of enhanced transmitted dosimetry to the relevant dielectric target pathways and of decreased power requirement are achieved.

An additional advantage of the present invention relates to enhanced effectiveness of pharmacological, chemical, cosmetic and topical agents as applied to, upon or on human, animal and plant cells, organs, tissues and molecules by accelerating the agents intended effects and improving efficacy.

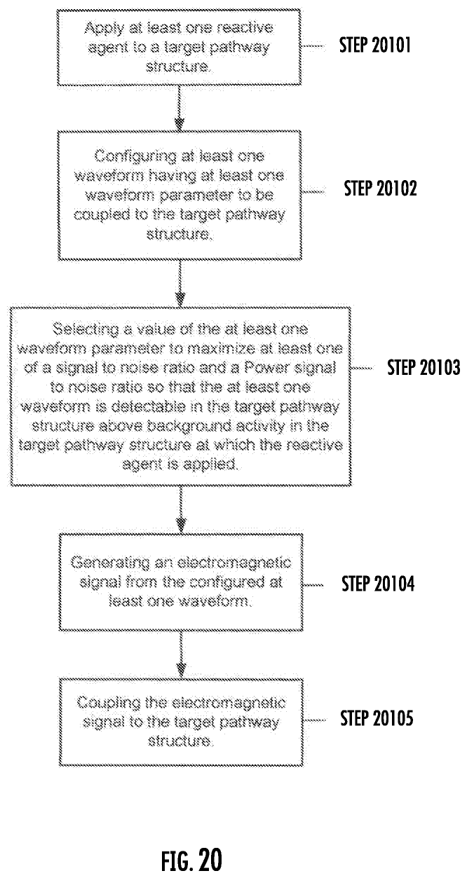

Referring to FIG. 20, wherein FIG. 20 is a flow diagram of a method according to an embodiment of the present invention, for enhancing effectiveness of pharmacological, chemical, cosmetic and topical agents used to treat stem cells, tissues, cells, organs, and molecules by delivering electromagnetic signals that can be pulsed, to target pathway structures such as ions and ligands of animals and humans, for therapeutic and prophylactic purposes. Target pathway structures can also include but are not limited to stem cells, tissues, cells, organs, and molecules Enhancing effectiveness of pharmacological, chemical, cosmetic and topical agents includes but is not limited to increased absorption rate, decreased effective dosages, faster delivery rates at an organism level; and increased binding kinetics and transport kinetics level at a molecular and cellular level. At least one reactive agent is applied to a target pathway structure (Step 20101). Reactive agents include but are not limited to pharmacological agents, chemical agents, cosmetic agents, topical agents, and genetic agents. Reactive agents can be ingested, applied topically, applied intravenously, intramuscularly, or by any other manner known within the medical community that causes interaction of substances with a target pathway structure, such as iontophoresis, X and light radiation, and heat. Pharmacological agents include but are not limited to antibiotics, growth factors, chemotherapeutic agents, antihistamines, Angiotensin inhibitors, beta blockers, statins, and anti-inflammatory drugs. Chemical agents include but are not limited to hydrogen peroxide, betadine, and alcohol. Topical agents include but are not limited to antibiotics, creams, retinol, benzoyl peroxide, tolnaftate, menthol, emollients, oils, lanolin, squalene, aloe vera, anti-oxidants, fatty acid, fatty acid ester, cod liver oil, alpha-tocopherol, petroleum, hydrogenated polybutene, vitamin A, vitamin E, topical proteins, and collagens. Cosmetic agents include but are not limited to make-up, eye-liner, and blush. Genetic agents include but are not limited to genes, DNA, and chromosomes.

Configuring at least one waveform having at least one waveform parameter to be coupled to the target pathway structure such as ions and ligands (Step 20102).

The at least one waveform parameter is selected to maximize at least one of a signal to noise ratio and a Power Signal to Noise ratio in a target pathway structure so that a waveform is detectable in the target pathway structure above its background activity (Step 20102) such as baseline thermal fluctuations in voltage and electrical impedance at a target pathway structure that depend upon a state of a cell and tissue, that is whether the state is at least one of resting, growing, replacing, and responding to injury to produce physiologically beneficial results. To be detectable in the target pathway structure the value of said at least one waveform parameter is chosen by using a constant of said target pathway structure to evaluate at least one of a signal to noise ratio, and a Power signal to noise ratio, to compare voltage induced by said at least one waveform in said target pathway structure to baseline thermal fluctuations in voltage and electrical impedance in said target pathway structure whereby bioeffective modulation occurs in said target pathway structure by said at least one waveform by maximizing said at least one of signal to noise ratio and Power signal to noise ratio, within a bandpass of said target pathway structure.

A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model (Step 20103). A repetitive electromagnetic signal can be generated for example inductively or capacitively, from said configured at least one waveform (Step 20104). The electromagnetic signal can also be non-repetitive. The electromagnetic signal is coupled to a target pathway structure such as ions and ligands by output of a coupling device such as an electrode or an inductor, placed in close proximity to the target pathway structure (Step 20105). Coupling of the electromagnetic signal to a target pathway structure can occur adjunctively, for example at any time prior to applying a reactive agent, at the same time a reactive agent is being applied, or after the time a reactive agent has been applied. The coupling enhances blood flow and modulation of binding of ions and ligands to regulatory molecules in molecules, tissues, cells, and organs thereby enhancing the reactive agents' bioeffectiveness.

In one embodiment, the method shown in FIG. 20 may be performed using a self-contained, lightweight, and portable apparatus such as that shown in FIG. 2. The apparatus is self-contained, lightweight, and portable. A miniature control circuit 33201 is coupled to an end of at least one connector 202 such as wire however the control circuit can also operate wirelessly. The opposite end of the at least one connector is coupled to a generating device such as an electrical coil 203. The miniature control circuit 201 is constructed in a manner that applies a mathematical model that is used to configure waveforms. The configured waveforms have to satisfy Power SNR so that for a given and known target pathway structure, it is possible to choose waveform parameters that satisfy Power SNR so that a waveform produces physiologically beneficial results, for example bioeffective modulation, and is detectable in the target pathway structure above its background activity.

In a preferred embodiment of the method of FIG. 20, a mathematical model is applied to induce a time-varying magnetic field and a time-varying electric field in a target pathway structure such as ions and ligands, comprising about 0.1 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency. A waveform configured using a preferred embodiment according to the present invention may be applied to a target pathway structure such as ions and ligands for a preferred total exposure time of under 1 minute to 240 minutes daily. However other exposure times can be used. Waveforms configured by the miniature control circuit 201 are directed to a generating device 203 such as electrical coils via connector 202. The generating device 203 delivers a pulsing magnetic field that can be used to provide treatment to a target pathway structure such as tissue. The miniature control circuit applies a pulsing magnetic field for a prescribed time and can automatically repeat applying the pulsing magnetic field for as many applications as are needed in a given time period, for example 10 times a day. The miniature control circuit can be configured to be programmable applying pulsing magnetic fields for any time repetition sequence. A preferred embodiment according to the present invention can enhance the pharmacological, chemical, cosmetic and topical agents' effectiveness by being incorporated into a positioning device 204, for example a bed. Coupling a pulsing magnetic field to a target pathway structure such as ions and ligands, therapeutically and prophylactically reduces inflammation thereby advantageously reducing pain, promoting healing in targeted areas, and enhancing interactions of pharmacological, chemical, cosmetic and topical agents with a target pathway structure. When electrical coils are used as the generating device 203, the electrical coils can be powered with a time varying magnetic field that induces a time varying electric field in a target pathway structure according to Faraday's law. An electromagnetic signal generated by the generating device 203 can also be applied using electrochemical coupling, wherein electrodes are in direct contact with skin or another outer electrically conductive boundary of a target pathway structure. In yet another embodiment according to the present invention, the electromagnetic signal generated by the generating device 203 can also be applied using electrostatic coupling wherein an air gap exists between a generating device 203 such as an electrode and a target pathway structure such as ions and ligands. An advantage of the preferred embodiment according to the present invention is that its ultra-lightweight coils and miniaturized circuitry allow for use with common physical therapy treatment modalities and at any for which growth, pain relief, and tissue and organ healing is desired. An advantageous result of application of the preferred embodiment according to the present invention is that tissue growth, repair, and maintenance can be accomplished and enhanced anywhere and at any time, for example while driving a car or watching television. Yet another advantageous result of application of the preferred embodiment is that growth, repair, and maintenance of molecules, cells, tissues, and organs can be accomplished and enhanced anywhere and at any time, for example while driving a car or watching television.

In the embodiment associated with FIG. 20, the electromagnetic signal may be coupled to a target pathway structure by a coupling device having the configuration shown in FIGS. 4A and B. In this embodiment, the coupling device 400 can be an electric coil 401 wound with single or multistrand flexible wire 402; however solid wire can also be used. In a preferred embodiment according to the present invention, the wire is made of copper but other materials can be used. The multistrand flexible magnetic wire 402 enables the electric coil 401 to conform to specific anatomical configurations such as a limb or joint of a human or animal. In an apparatus for wound treatment, the electric coil 401 may comprise about 1 to about 1000 turns of about 0.01 mm to about 0.1 mm diameter at least one of single magnet wire and multistrand magnet wire, wound on an initially circular form having an outer diameter between about 2.5 cm and about 50 cm but other numbers of turns and wire diameters can be used. The electric coil 26401 can be encased with a non-toxic PVC mould 403 but other non-toxic moulds can also be used. The electric coil can also be incorporated in dressings, bandages, garments, and other structures typically used for wound treatment.

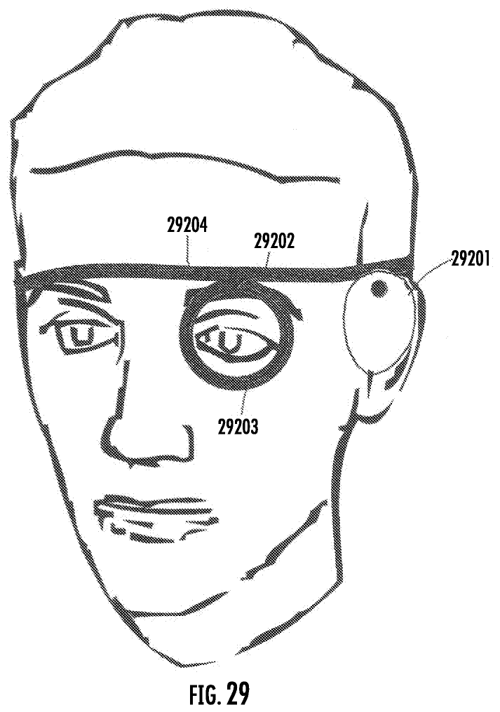

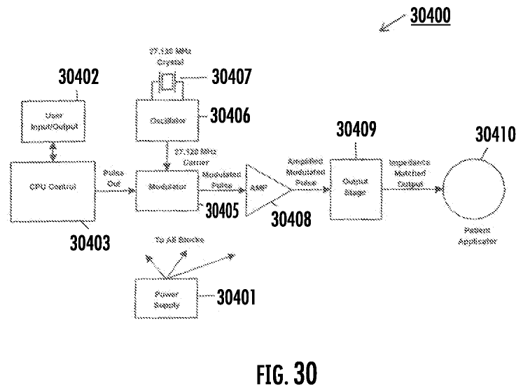

An embodiment of the method associated with FIG. 30 may employ a waveform having the parameters described above in connection with FIG. 5, but other waveforms, envelopes, and burst periods that follow a mathematical model such as SNR and Power SNR, may be used.

In other embodiments, an apparatus for performing the method of FIG. 19 may also incorporated into a positioning device such as the wrist support 600 shown in FIG. 6, or other devices such as knee, elbow, lower back, shoulder, other anatomical wraps, and apparel such as garments, fashion accessories, and footwear. Alternatively, the apparatus may be incorporated into the mattress pad of FIG. 14.

### EXAMPLE 6

This study determined to what extent treatment with pulsed electromagnetic frequency (“PEMF”) waveforms affects blood perfusion in a treated region. All testing was done in a temperature controlled room (23 to 24° C.) with the subject seated on a comfortable easy chair. On each arm a non-metallic laser Doppler probe was affixed with double-sided tape to a medial forearm site approximately 5 cm distal to the antecubital space. A temperature sensing thermistor for surface temperature measurements was placed approximately 1 cm distal to the outer edge of the probes and secured with tape. A towel was draped over each forearm to diminish the direct effects of any circulating air currents. With the subject resting comfortably, the skin temperature of each arm was monitored. During this monitoring interval the excitation coil for producing the PEMF waveform according to the instant invention was positioned directly above the Laser Doppler probe of the right forearm at a vertical distance of approximately 2 cm from the skin surface. When the monitored skin temperature reached a steady state value, the data acquisition phase was begun. This consisted of a 20 minute baseline interval followed by a 45 minute interval in which the PEMF waveform was applied.

Skin temperature was recorded at five minute intervals during the entire protocol. Blood perfusion signals as determined with the Laser Doppler Flowmeter (“LDF”) were continuously displayed on a chart recorder and simultaneously acquired by a computer following analog to digital conversion. The LDF signals were time averaged by the computer during each contiguous five minute interval of measurement to produce a single averaged perfusion value for each interval. At the end of the procedure the relative magnetic field strength at the skin site was measured with a 1 cm diameter loop which was coupled to a specially designed and calibrated metering system.

For each subject the baseline perfusion for the treated arm and the control arm was determined as the average during the 20 minute baseline interval. Subsequent perfusion values, following the start of PEMF treatment, was expressed as a percentage of this baseline. Comparison between the treated and control arms were done using analysis of variance with arm (treated vs. control) as the grouping variables and with time as a repeated measure.

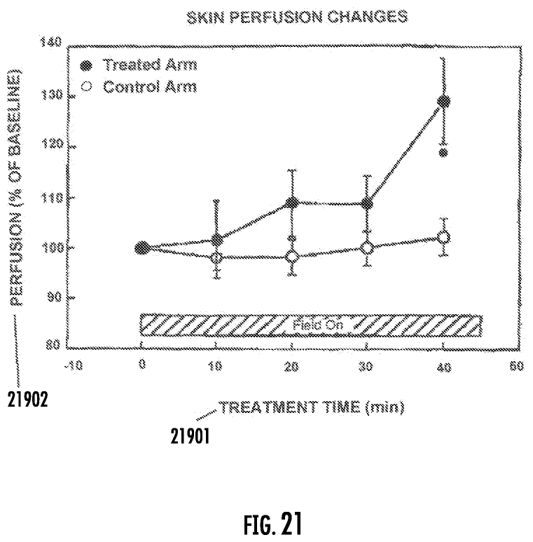

FIG. 21 summarizes the time course of the perfusion change found during treatment for the nine subjects studied with time being plotted on the x-axis 40901 and perfusion on the y-axis 40902. Analysis shows significant treatment-time interaction (p=0.03) with a significantly (p<0.01) elevated blood perfusion in the treated arm after 40 minutes of PEMF treatment. The absolute values of baseline perfusion (my) did not differ between control and treated arms. Analysis of covariance with the baseline perfusion in absolute units (my) as the covariate also shows an overall difference between treated and control arms (p<0.01).

A main finding of the present investigational study is that PEMF treatment, when applied in the manner described, is associated with a significant augmentation in their resting forearm skin microvascular perfusion. This augmentation, which averages about 30% as compared with resting pre-treatment levels, occurs after about 40 minutes of treatment whereas no such augmentation is evident in the contralateral non-treated arm. This allows the increased flow of pharmacological, chemical, topical, cosmetic, and genetic agents to the intended tissue target.

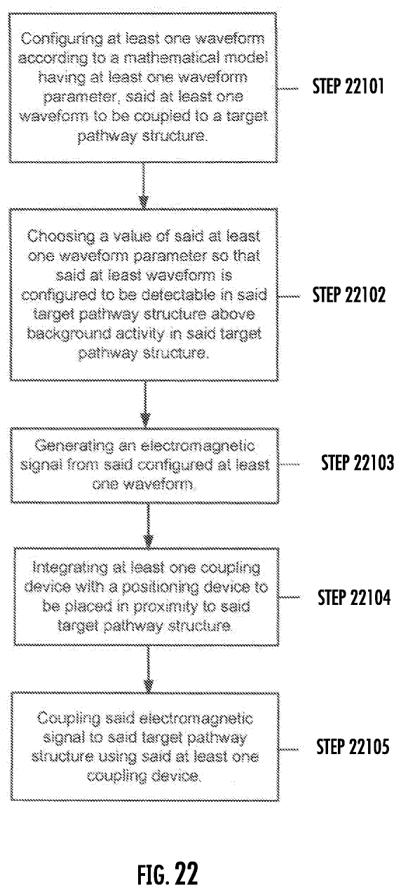

Referring to FIG. 22, wherein FIG. 2 is a flow diagram of a method for delivering electromagnetic signals to tissue target pathway structures such as ions and ligands of animals, and humans for therapeutic and prophylactic purposes according to an embodiment of the present invention. A mathematical model having at least one waveform parameter is applied to configure at least one waveform to be coupled to target pathway structures such as ions and ligands (Step 2201). The configured waveform satisfies a Power SNR model so that for a given and known target pathway structure it is possible to choose at least one waveform parameter so that a waveform is detectable in the target pathway structure above its background activity (Step 22102) such as baseline thermal fluctuations in voltage and electrical impedance at a target pathway structure that depend upon a state of a cell and tissue, that is whether the state is at least one of resting, growing, replacing, and responding to injury.

A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model (Step 22102). A repetitive electromagnetic signal can be generated for example inductively or capacitively, from said configured at least one waveform (Step 22103). The electromagnetic signal is coupled to a target pathway structure such as ions and ligands by output of a coupling device such as an electrode or an inductor, placed in close proximity to the target pathway structure (Step 2104) using a positioning device by integrating the coupling device with the positioning device (Step 2105). The coupling enhances modulation of binding of ions and ligands to regulatory molecules tissues, cells, and organs. The coupling device can be integrated into the structure of the positioning device. The positioning device can be surgical dressings, wound dressings, pads, seat cushions, mattress pads, shoes, wheelchairs, chairs, and any other garment and structure that can be juxtaposed to living tissue and cells. In addition, the positioning device can include at least one of a therapeutic surface, a therapeutic structure, and a therapeutic device, such as diathermy, ultrasound, TENS, massage, heat compress, cold compress, anatomical support surfaces, structures, and devices. An advantage of integrating the coupling device with a positioning device is that therapeutic treatment can be administered in an unnoticeable fashion and can be administered anywhere and at any time.

In one embodiment, the method shown in FIG. 22 may be performed using a self-contained, lightweight, and portable apparatus such as that shown in FIG. 2. The configured waveforms have to satisfy Power SNR so that for a given and known target pathway structure, it is possible to choose waveform parameters that satisfy Power SNR so that a waveform produces physiologically beneficial results, for example bioeffective modulation, and is detectable in the target pathway structure above its background activity.

In a preferred embodiment of the method of FIG. 22 according to the present invention, a mathematical model is applied to induce a time-varying magnetic field and a time-varying electric field in a target pathway structure such as ions and ligands, comprising about 0.1 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency. In a preferred embodiment, a waveform configured having the same parameters discussed above in connection with FIG. 5 may be applied to a target pathway structure such as ions and ligands for a preferred total exposure time of under 1 minute to 240 minutes daily. However other exposure times can be used. Waveforms configured by the miniature control circuit 201, which may be similar or identical to the control circuit 300 shown in FIG. 3, are directed to a generating device 203 such as electrical coils via connector 202. The generating device 203 delivers a pulsing magnetic field that can be used to provide treatment to a target pathway structure such as tissue. The miniature control circuit applies a pulsing magnetic field for a prescribed time and can automatically repeat applying the pulsing magnetic field for as many applications as are needed in a given time period, for example 10 times a day. The miniature control circuit can be configured to be programmable applying pulsing magnetic fields for any time repetition sequence.

A preferred embodiment according to the present invention can be positioned to treat hair by being incorporated with a positioning device thereby making the unit self-contained. Coupling a pulsing magnetic field to a target pathway structure such as ions and ligands, therapeutically and prophylactically reduces inflammation thereby reducing pain and promotes healing in treatment areas. When electrical coils are used as the generating device 203, the electrical coils can be powered with a time varying magnetic field that induces a time varying electric field in a target pathway structure according to Faraday's law. An electromagnetic signal generated by the generating device 203 can also be applied using electrochemical coupling, wherein electrodes are in direct contact with skin or another outer electrically conductive boundary of a target pathway structure.

In yet another embodiment according to the present invention, the electromagnetic signal generated by the generating device 203 can also be applied using electrostatic coupling wherein an air gap exists between a generating device 203 such as an electrode and a target pathway structure such as ions and ligands. An advantage of the preferred embodiment according to the present invention is that its ultra-lightweight coils and miniaturized circuitry allow for use with common physical therapy treatment modalities and at any location for which tissue growth, pain relief, and tissue and organ healing is desired. An advantageous result of application of the preferred embodiment according to the present invention is that tissue growth, repair, and maintenance can be accomplished and enhanced anywhere and at any time. Yet another advantageous result of application of the preferred embodiment is that growth, repair, and maintenance of molecules, cells, tissues, and organs can be accomplished and enhanced anywhere and at any time.

### EXAMPLE 7

This example illustrates the effects of PRF electromagnetic fields chosen via the Power SNR method on neurons in culture.

Primary cultures were established from embryonic days 15-16 rodent mesencephalon. This area is dissected, dissociated into single cells by mechanical trituration, and cells are plated in either defined medium or medium with serum. Cells are typically treated after 6 days of culture, when neurons have matured and developed mechanisms that render them vulnerable to biologically relevant toxins. After treatment, conditioned media is collected.

Enzyme linked immunosorbent assays (“ELISAs”) for growth factors such as Fibroblast Growth Factor beta (“FGFb”) are used to quantify their release into the medium. Dopaminergic neurons are identified with an antibody to tyrosine hydroxylase (“TH”), an enzyme that converts the amino acid tyrosine to L-dopa, the precursor of dopamine, since dopaminergic neurons are the only cells that produce this enzyme in this system. Cells are quantified by counting TH+ cells in perpendicular strips across the culture dish under 100× magnification.

Serum contains nutrients and growth factors that support neuronal survival. Elimination of serum induces neuronal cell death. Culture media was changed and cells were exposed to PMF (power level 6, burst width 3000 μsec, and frequency 1 Hz). Four groups were utilized. Group 1 used No PMF exposure (null group). Group 2 used Pre-treatment (PMF treatment 2 hours before medium change). Group 3 used Post-treatment (PMF treatment 2 hours after medium change). Group 4 used Immediate treatment (PMF treatment simultaneous to medium change).

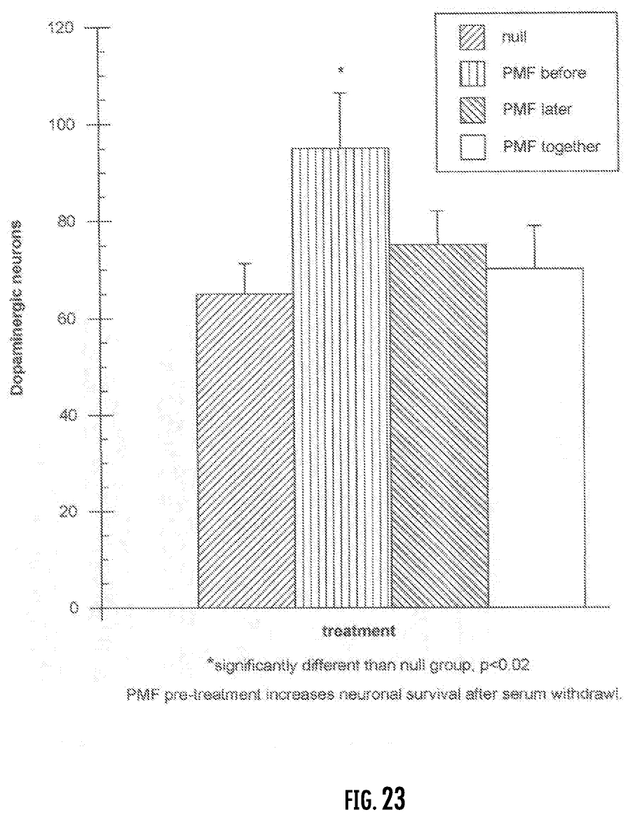

Results demonstrate a 46% increase in the numbers of surviving dopaminergic neurons after 2 days when cultures were exposed to PMF prior to serum withdrawal. Other treatment regimes had no significant effects on numbers of surviving neurons. The results are shown in FIG. 23 where type of treatment is shown on the x-axis and number of neurons is shown on the y-axis.

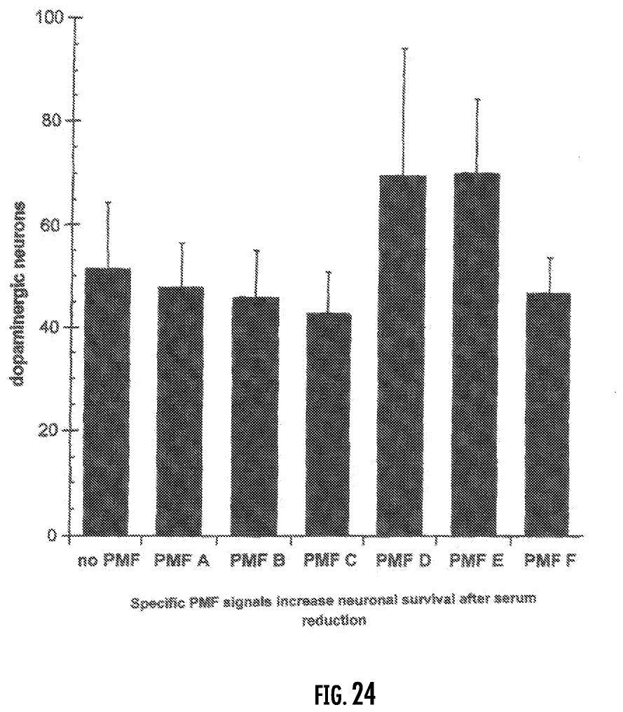

FIG. 24, where treatment is shown on the x-axis and number of neurons is shown on the y-axis, illustrates that PMF signals D and E increase numbers of dopaminergic neurons after reducing serum concentrations in the medium by 46% and 48% respectively. Both signals were configured with a burst width of 3000 μsec, and the repetition rates are 5/sec and 1/sec, respectively. Notably, signal D was administered in a chronic paradigm in this experiment, but signal E was administered only once: 2 hours prior to serum withdrawal, identical to experiment 1 (see above), producing effects of the same magnitude (46% vs. 48%). Since the reduction of serum in the medium reduces the availability of nutrients and growth factors, PMF induces the synthesis or release of these factors by the cultures themselves.

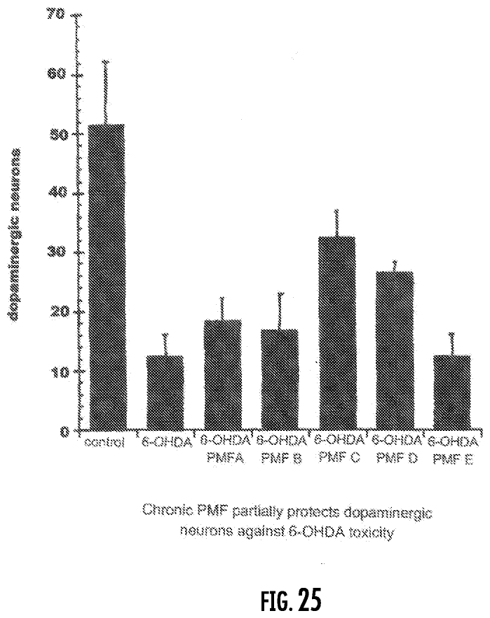

This portion of the experiment was performed to illustrate the effects of PMF toxicity induced by 6-OHDA, producing a well-characterized mechanism of dopaminergic cell death. This molecule enters cells via high affinity dopamine transporters and inhibits mitochondrial enzyme complex I, thus killing these neurons by oxidative stress. Cultures were treated with 25 μM 6-OHDA after chronic, or acute PMF exposure paradigms. FIG. 25 illustrates these results, where treatment is shown on the x-axis and number of neurons is shown on the y-axis. The toxin killed approximately 80% of the dopaminergic neurons in the absence of PMF treatment. One dose of PMF (power=6; burst width=3000 μsec; frequency=1/sec) significantly increased neuronal survival over 6-OHDA alone (2.6-fold; p<0.02). This result has particular relevance to developing neuroprotection strategies for Parkinson's disease, because 6-OHDA is used to lesion dopaminergic neurons in the standard rodent model of Parkinson's disease, and the mechanism of toxicity is similar in some ways to the mechanism of neurodegeneration in Parkinson's disease itself.

### EXAMPLE 8

In this example electromagnetic field energy was used to stimulate neovascularization in an in vivo model. Two different signals were employed, one configured according to prior art and a second configured according to an embodiment of the present invention.

One hundred and eight Sprague-Dawley male rats weighing approximately 300 grams each, were equally divided into nine groups. All animals were anesthetized with a mixture of ketamine/acepromazine/Stadol at 0.1 cc/g. Using sterile surgical techniques, each animal had a 12 cm to 14 cm segment of tail artery harvested using microsurgical technique. The artery was flushed with 60 U/ml of heparinized saline to remove any blood or emboli.

These tail vessels, with an average diameter of 0.4 mm to 0.5 mm, were then sutured to the transected proximal and distal segments of the right femoral artery using two end-to-end anastomoses, creating a femoral arterial loop. The resulting loop was then placed in a subcutaneous pocket created over the animal's abdominal wall/groin musculature, and the groin incision was closed with 4-0 Ethilon. Each animal was then randomly placed into one of nine groups: groups 1 to 3 (controls), these rats received no electromagnetic field treatments and were killed at 4, 8, and 12 weeks; groups 4 to 6, 30 min. treatments twice a day using 0.1 gauss electromagnetic fields for 4, 8, and 12 weeks (animals were killed at 4, 8, and 12 weeks, respectively); and groups 7 to 9, 30 min. treatments twice a day using 2.0 gauss electromagnetic fields for 4, 8, and 12 weeks (animals were killed at 4, 8, and 12 weeks, respectively).

Pulsed electromagnetic energy was applied to the treated groups using a device constructed according to an embodiment of the present invention Animals in the experimental groups were treated for 30 minutes twice a day at either 0.1 gauss or 2.0 gauss, using short pulses (2 msec to 20 msec) 27.12 MHz. Animals were positioned on top of the applicator head and confined to ensure that treatment was properly applied. The rats were reanesthetized with ketamine/acepromazine/Stadol intraperitoneally and 100 U/kg of heparin intravenously. Using the previous groin incision, the femoral artery was identified and checked for patency. The femoral/tail artery loop was then isolated proximally and distally from the anastomoses sites, and the vessel was clamped off. Animals were then killed. The loop was injected with saline followed by 0.5 cc to 1.0 cc of colored latex through a 25-gauge cannula and clamped. The overlying abdominal skin was carefully resected, and the arterial loop was exposed. Neovascularization was quantified by measuring the surface area covered by new blood-vessel formation delineated by the intraluminal latex. All results were analyzed using the SPSS statistical analysis package.

The most noticeable difference in neovascularization between treated versus untreated rats occurred at week 4. At that time, no new vessel formation was found among controls, however, each of the treated groups had similar statistically significant evidence of neovascularization at 0 cm2 versus 1.42±0.80 cm² (p<0.001). These areas appeared as a latex blush segmentally distributed along the sides of the arterial loop. At 8 weeks, controls began to demonstrate neovascularization measured at 0.7±0.82 cm2. Both treated groups at 8 weeks again had approximately equal statistically significant (p<0.001) outcroppings of blood vessels of 3.57±1.82 cm² for the 0.1 gauss group and of 3.77±1.82 cm² for the 2.0 gauss group. At 12 weeks, animals in the control group displayed 1.75±0.95 cm² of neovascularization, whereas the 0.1 gauss group demonstrated 5.95±3.25 cm², and the 2.0 gauss group showed 6.20±3.95 cm² of arborizing vessels. Again, both treated groups displayed comparable statistically significant findings (p<0.001) over controls.

These experimental findings demonstrate that electromagnetic field stimulation of an isolated arterial loop according to an embodiment of the present invention increases the amount of quantifiable neovascularization in an in vivo rat model. Increased angiogenesis was demonstrated in each of the treated groups at each of the sacrifice dates. No differences were found between the results of the two gauss levels tested as predicted by the teachings of the present invention.

Induced time-varying currents from PEMF or PRF devices flow in a hair and cerebrofacial target pathway structure such as a molecule, cell, tissue, and organ, and it is these currents that are a stimulus to which cells and tissues can react in a physiologically meaningful manner The electrical properties of a hair and cerebrofacial target pathway structure affect levels and distributions of induced current. Molecules, cells, tissue, and organs are all in an induced current pathway such as cells in a gap junction contact. Ion or ligand interactions at binding sites on macromolecules that may reside on a membrane surface are voltage dependent processes, that is electrochemical, that can respond to an induced electromagnetic field (“E”). Induced current arrives at these sites via a surrounding ionic medium. The presence of cells in a current pathway causes an induced current (“J”) to decay more rapidly with time (“J(t)”). This is due to an added electrical impedance of cells from membrane capacitance and time constants of binding and other voltage sensitive membrane processes such as membrane transport.

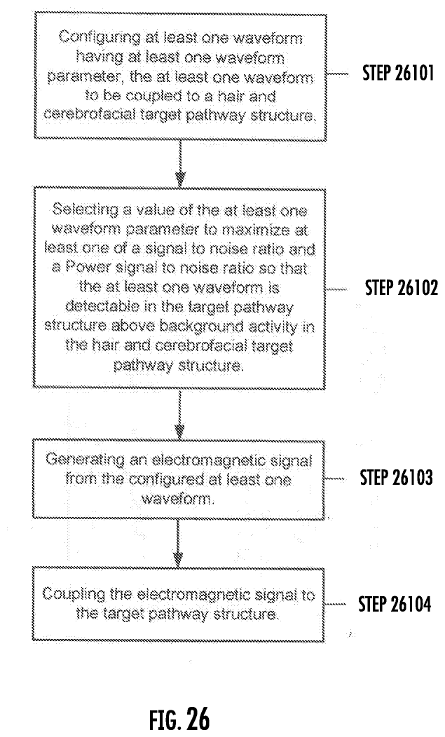

Referring to FIG. 26, wherein FIG. 26 is a flow diagram of a method for delivering electromagnetic signals that can be pulsed, to hair and cerebrofacial tissue target pathway structures such as ions and ligands of animals, and humans for therapeutic and prophylactic purposes according to an embodiment of the present invention.

At least one waveform having at least one waveform parameter is configured to be coupled to hair and cerebrofacial target pathway structures such as ions and ligands (Step 26101). Hair and cerebrofacial target pathway structures are located in a cerebrofacial treatment area. Examples of a cerebrofacial treatment area include but are not limited to, hair, a brain, sinuses, adenoids, tonsils, eyes, a nose, ears, teeth, and a tongue.

The at least one waveform parameter is selected to maximize at least one of a signal to noise ratio and a Power Signal to Noise ratio in a hair and cerebrofacial target pathway structure so that a waveform is detectable in the hair and cerebofacial target pathway structure above its background activity (Step 26102) such as baseline thermal fluctuations in voltage and electrical impedance at a target pathway structure that depend upon a state of a cell and tissue, that is whether the state is at least one of resting, growing, replacing, and responding to injury to produce physiologically beneficial results. To be detectable in the hair and cerebrofacial target pathway structure the value of said at least one waveform parameter is chosen by using a constant of said target pathway structure to evaluate at least one of a signal to noise ratio, and a Power signal to noise ratio, to compare voltage induced by said at least one waveform in said target pathway structure to baseline thermal fluctuations in voltage and electrical impedance in said target pathway structure whereby bioeffective modulation occurs in said target pathway structure by said at least one waveform by maximizing said at least one of signal to noise ratio and Power signal to noise ratio, within a bandpass of said target pathway structure.

A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model (Step 26102). A repetitive electromagnetic signal can be generated for example inductively or capacitively, from said configured at least one waveform (Step 26103). The electromagnetic signal can also be non-repetitive. The electromagnetic signal is coupled to a hair and cerebrofacial target pathway structure such as ions and ligands by output of a coupling device such as an electrode or an inductor, placed in close proximity to the target pathway structure (Step 26104). The coupling enhances modulation of binding of ions and ligands to regulatory molecules in hair and other cerebrofacial molecules, tissues, cells, and organs.

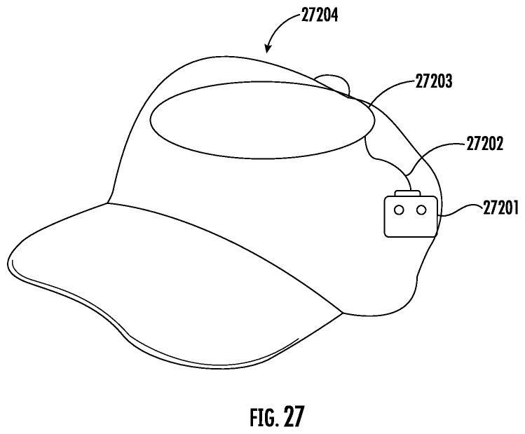

FIG. 27 illustrates a preferred embodiment of an apparatus according to the present invention. The apparatus is self-contained, lightweight, and portable. A miniature control circuit 27201 is coupled to an end of at least one connector 27202 such as wire however the control circuit can also operate wirelessly. The opposite end of the at least one connector is coupled to a generating device such as an electrical coil 27203. The miniature control circuit 27201 is constructed in a manner that applies a mathematical model that is used to configure waveforms. The configured waveforms have to satisfy Power SNR so that for a given and known hair and cerebrofacial target pathway structure, it is possible to choose waveform parameters that satisfy Power SNR so that a waveform produces physiologically beneficial results, for example bioeffective modulation, and is detectable in the hair and cerebrofacial target pathway structure above its background activity.

A preferred embodiment according to the present invention applies a mathematical model to induce a time-varying magnetic field and a time-varying electric field in a hair and cerebrofacial target pathway structure such as ions and ligands, comprising about 0.1 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency. A waveform having the parameters shown in FIG. 5 may be applied to a hair and cerebrofacial target pathway structure such as ions and ligands for a preferred total exposure time of under 1 minute to 240 minutes daily. However other exposure times can be used. Waveforms configured by the miniature control circuit 27201, which may be similar or identical to the control circuit 300 shown in FIG. 3, are directed to a generating device 27203 such as electrical coils via connector 27202. The generating device 49203 delivers a pulsing magnetic field that can be used to provide treatment to a hair and cerebrofacial target pathway structure such as hair tissue. The miniature control circuit applies a pulsing magnetic field for a prescribed time and can automatically repeat applying the pulsing magnetic field for as many applications as are needed in a given time period, for example 10 times a day. The miniature control circuit 300 can be constructed to be programmable and apply a pulsing magnetic field for a prescribed time and to automatically repeat applying the pulsing magnetic field for as many applications as are needed in a given time period, for example 10 times a day. A preferred embodiment according to the present invention uses treatments times of about 10 minutes to about 30 minutes.

A preferred embodiment according to the present invention can be positioned to treat hair by being incorporated into a positioning device such as a hat 27204, thereby making the unit self-contained. Coupling a pulsing magnetic field to a hair and cerebrofacial target pathway structure such as ions and ligands, therapeutically and prophylactically reduces inflammation thereby advantageously reducing pain and promoting healing in cerebrofacial areas. When electrical coils are used as the generating device 27203, the electrical coils can be powered with a time varying magnetic field that induces a time varying electric field in a target pathway structure according to Faraday's law. An electromagnetic signal generated by the generating device 27203 can also be applied using electrochemical coupling, wherein electrodes are in direct contact with skin or another outer electrically conductive boundary of a hair and cerebrofacial target pathway structure.

In yet another embodiment according to the present invention, the electromagnetic signal generated by the generating device 27203 can also be applied using electrostatic coupling wherein an air gap exists between a generating device 27203 such as an electrode and a hair and cerebrofacial target pathway structure such as ions and ligands. An advantage of the preferred embodiment according to the present invention is that its ultra-lightweight coils and miniaturized circuitry allow for use with common physical therapy treatment modalities and at any cerebrofacial location for which hair growth, pain relief, and tissue and organ healing is desired. An advantageous result of application of the preferred embodiment according to the present invention is that hair growth, repair, and maintenance can be accomplished and enhanced anywhere and at any time, for example while driving a car or watching television. Yet another advantageous result of application of the preferred embodiment is that growth, repair, and maintenance of cerebrofacial molecules, cells, tissues, and organs can be accomplished and enhanced anywhere and at any time, for example while driving a car or watching television.

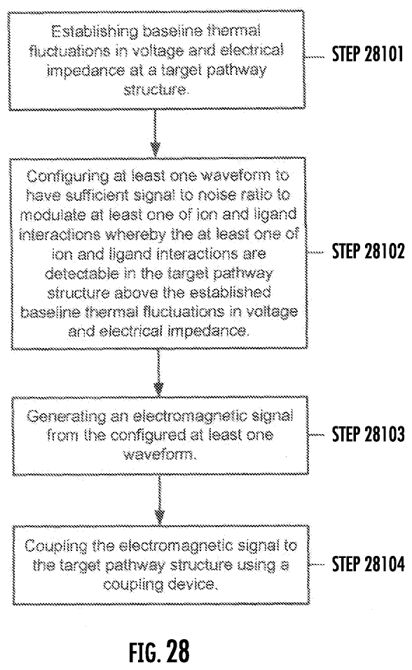

Referring to FIG. 28 wherein FIG. 28 is a flow diagram of a method for generating electromagnetic signals to be coupled to an eye according to an embodiment of the present invention, a target pathway structure such as ions and ligands, is identified. Establishing a baseline background activity such as baseline thermal fluctuations in voltage and electrical impedance, at the target pathway structure by determining a state of at least one of a cell and a tissue at the target pathway structure, wherein the state is at least one of resting, growing, replacing, and responding to injury. (STEP 28101) The state of the at least one of a cell and a tissue is determined by its response to injury or insult. Configuring at least one waveform to have sufficient signal to noise ratio to modulate at least one of ion and ligand interactions whereby the at least one of ion and ligand interactions are detectable in the target pathway structure above the established baseline thermal fluctuations in voltage and electrical impedance. (STEP 28102) Generating an electromagnetic signal from the configured at least one waveform. (STEP 28103) The electromagnetic signal can be generated by using at least one waveform configured by applying a mathematical model such as an equation, formula, or function having at least one waveform parameter that satisfies an SNR or Power SNR mathematical model such that ion and ligand interactions are modulated and the at least one configured waveform is detectable at the target pathway structure above its established background activity. Coupling the electromagnetic signal to the target pathway structure using a coupling device. (STEP 28104) The generated electromagnetic signals can be coupled for therapeutic and prophylactic purposes. Since ophthalmic tissue is very delicate, application of electromagnetic signals using an embodiment according to the present invention is extremely safe and efficient since the application of electromagnetic signals is non-invasive.

FIG. 29 illustrates a preferred embodiment of an apparatus according to the present invention. The apparatus is self-contained, lightweight, and portable. A miniature control circuit 29201 is coupled to an end of at least one connector 29202 such as wire however the control circuit can also operate wirelessly. The opposite end of the at least one connector is coupled to a generating device such as an electrical coil 29203. The miniature control circuit 29201 is constructed in a manner that applies a mathematical model that is used to configure waveforms. The configured waveforms have to satisfy a Power SNR model so that for a given and known target pathway structure, it is possible to choose waveform parameters that satisfy Power SNR so that a waveform is detectable in the target pathway structure above its background activity.

A preferred embodiment according to the present invention applies a mathematical model to induce a time-varying magnetic field and a time-varying electric field in a target pathway structure such as ions and ligands, comprising about 0.001 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified function where f=frequency. A waveform having the parameters described earlier in connection with FIG. 5 may be applied to a target pathway structure such as ions and ligands for a preferred total exposure time of under 1 minute to 240 minutes daily. However other exposure times can be used. Waveforms configured by the miniature control circuit 29201 which may be similar or identical to the control circuit 300 shown in FIG. 3 are directed to a generating device 29203 such as electrical coils via connector 29202. The generating device 29203 delivers a pulsing magnetic field configured according to a mathematical model that can be used to provide treatment to a target pathway structure such as eye tissue. The miniature control circuit applies a pulsing magnetic field for a prescribed time and can automatically repeat applying the pulsing magnetic field for as many applications as are needed in a given time period, for example 10 times a day. The miniature control circuit can be configured to be programmable applying pulsing magnetic fields for any time repetition sequence. A preferred embodiment according to the present invention uses treatments times of about 1 minute to about 30 minutes.

A preferred embodiment according to the present invention can be positioned to treat ophthalmic tissue by being incorporated with a positioning device 29204 such as an eye-patch, eyeglasses, goggles, and monocles thereby making the unit self-contained. Coupling a pulsing magnetic field to a target pathway structure such as ions and ligands, therapeutically and prophylactically reduces inflammation thereby reducing pain and promotes healing in treatment areas. When electrical coils are used as the generating device 29203, the electrical coils can be powered with a time varying magnetic field that induces a time varying electric field in a target pathway structure according to Faraday's law. An electromagnetic signal generated by the generating device 2903 can also be applied using electrochemical coupling, wherein electrodes are in direct contact with skin or another outer electrically conductive boundary of a target pathway structure.

In yet another embodiment according to the present invention, the electromagnetic signal generated by the generating device 29203 can also be applied using electrostatic coupling wherein an air gap exists between a generating device 56203 such as an electrode and a target pathway structure such as ions and ligands. An advantage of the preferred embodiment according to the present invention is that its ultra-lightweight coils and miniaturized circuitry allow for use with common physical therapy treatment modalities and at any location for which tissue growth, pain relief, and tissue and organ healing is desired. An advantageous result of application of the preferred embodiment according to the present invention is that tissue growth, repair, and maintenance can be accomplished and enhanced anywhere and at any time. Yet another advantageous result of application of the preferred embodiment is that growth, repair, and maintenance of molecules, cells, tissues, and organs can be accomplished and enhanced anywhere and at anytime. A preferred embodiment according to the present invention delivers PEMF for application to ophthalmic tissue that is infected with diseases as macular degeneration, glaucoma, retinosa pigmentosa, repair and regeneration of optic nerve prophylaxis, and other related diseases.

FIG. 30 depicts a block diagram of an embodiment according to the present invention of a miniature control circuit 30400. The miniature control circuit 30400 produces waveforms that drive a generating device such as wire coils described above in FIG. 29 The miniature control circuit can be activated by any activation means such as an on/off switch. The miniature control circuit 30400 has a power source such as a lithium battery 30401. In another embodiment according to the present invention the power source can be an external power source such as an electric current outlet such as an AC/DC outlet, coupled to the present invention for example by a plug and wire. A user input/output means 30402 such as an on/off switch controls voltage to the miniature control circuit and is connected to a cpu-control 30403. The cpu-control 30403 creates a SNR EMF waveform by processing information provided to it via flash memory programmed having SNR EMF signal parameters such as pulse shape, burst width, burst envelope shape, and burst repetition rate. The waveform is pulse modulated by a modulator 30405 interfacing with an oscillator 30406 having a crystal 30407 controlled by the cpu-control 30403 according to the SNR EMF signal parameters programmed into the flash memory of the cpu-control 30403. The oscillator 30406 having a crystal 30406 provides a carrier frequency. A preferred embodiment of the crystal is a 27.120 MHz crystal but other MHz crystals can be used. The modulated waveform, which may have the same parameters of the waveform described in connection with FIG. 5, is then amplified by an amp 30408 and sent to an output stage means 30409 where the amplified modulated waveform is matched to impedance via an RC circuit across a patient applicator 30410 such as a coil. The patient applicator generates a SNR EMF signal to be delivered to a patient.

According to yet another embodiment of the present invention by applying a high spectral density voltage envelope as a modulating or pulse-burst defining parameter, power requirements for such amplitude modulated pulse bursts can be significantly lower than that of an unmodulated pulse burst containing pulses within a similar frequency range. This is due to a substantial reduction in duty cycle within repetitive burst trains brought about by imposition of an irregular amplitude and preferably a random amplitude onto what would otherwise be a substantially uniform pulse burst envelope. Accordingly, the dual advantages, of enhanced transmitted dosimetry to the relevant dielectric pathways and of decreased power requirement are achieved.

Referring to FIG. 31 wherein FIG. 31 is a flow diagram of a method for generating electromagnetic signals to be coupled to a respiratory target pathway structure according to an embodiment of the present invention, a target pathway structure such as ions and ligands, is identified. Establishing a baseline background activity such as baseline thermal fluctuations in voltage and electrical impedance, at the target pathway structure by determining a state of at least one of a cell and a tissue at the target pathway structure, wherein the state is at least one of resting, growing, replacing, and responding to injury. (STEP 31101) The state of the at least one of a cell and a tissue is determined by its response to injury or insult. Configuring at least one waveform to have sufficient signal to noise ratio to modulate at least one of ion and ligand interactions whereby the at least one of ion and ligand interactions are detectable in the target pathway structure above the established baseline thermal fluctuations in voltage and electrical impedance. (STEP 31102) Repetitively generating an electromagnetic signal from the configured at least one waveform. (STEP 31103) The electromagnetic signal can be generated by using at least one waveform configured by applying a mathematical model such as an equation, formula, or function having at least one waveform parameter that satisfies an SNR or Power SNR mathematical model such that ion and ligand interactions are modulated and the at least one configured waveform is detectable at the target pathway structure above its established background activity. Coupling the electromagnetic signal to the target pathway structure using a coupling device. (STEP 31104) The generated electromagnetic signals can be coupled for therapeutic and prophylactic purposes. The coupling enhances a stimulus that cells and tissues react to in a physiological meaningful manner for example, treatment of lung diseases resulting from inflammatory processes caused by inhalation of foreign material into lung tissue. Since lung tissue is very delicate, application of electromagnetic signals using an embodiment according to the present invention is extremely safe and efficient since the application of electromagnetic signals is non-invasive.

In an aspect of the present invention, a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model. A repetitive electromagnetic signal can be generated for example inductively or capacitively, from the configured at least one waveform. The electromagnetic signal is coupled to a target pathway structure such as ions and ligands by output of a coupling device such as an electrode or an inductor, placed in close proximity to the target pathway structure using a positioning device. The coupling enhances modulation of binding of ions and ligands to regulatory molecules, tissues, cells, and organs. According to an embodiment of the present invention EMF signals configured using SNR analysis to match the bandpass of a second messenger whereby the EMF signals can act as a first messenger to modulate biochemical cascades such as production of cytokines, Nitric Oxide, Nitric Oxide Synthase and growth factors that are related to tissue growth and repair. A detectable E field amplitude is produced within a frequency response of Ca2+ binding.

FIG. 32 illustrates an embodiment of an apparatus according to the present invention. The apparatus is self-contained, lightweight, and portable. A miniature control circuit 32201, which may be similar or identical to the control circuit 300 shown in FIG. 3, is connected to a generating device such as an electrical coil 32202. The miniature control circuit 32201 is constructed in a manner that applies a mathematical model that is used to configure waveforms. The configured waveforms have to satisfy a Power SNR model so that for a given and known target pathway structure, it is possible to choose waveform parameters that satisfy Power SNR so that a waveform is detectable in the target pathway structure above its background activity. An embodiment according to the present invention applies a mathematical model to induce a time-varying magnetic field and a time-varying electric field in a target pathway structure such as ions and ligands, comprising about 0.001 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency. A waveform configured using an embodiment according to the present invention may be applied to a target pathway structure such as ions and ligands, preferably for a total exposure time of under 1 minute to 240 minutes daily. However other exposure times can be used. Waveforms configured by the miniature control circuit 32201, which may be similar or identical to the control circuit 300 shown in FIG. 3, are directed to a generating device 32202 such as electrical coils. Preferably, the generating device 32202 is a conformable coil for example pliable, comprising one or more turns of electrically conducting wire in a generally circular or oval shape however other shapes can be used. The generating device 32202 delivers a pulsing magnetic field configured according to a mathematical model that can be used to provide treatment to a target pathway structure such as lung tissue. The miniature control circuit applies a pulsing magnetic field for a prescribed time and can automatically repeat applying the pulsing magnetic field for as many applications as are needed in a given time period, for example 12 times a day. The miniature control circuit can be configured to be programmable applying pulsing magnetic fields for any time repetition sequence. An embodiment according to the present invention can be positioned to treat respiratory tissue by being incorporated with a positioning device such as a bandage or a vest thereby making the unit self-contained. Coupling a pulsing magnetic field to a target pathway structure such as ions and ligands, therapeutically and prophylactically reduces inflammation thereby reducing pain and promotes healing in treatment areas. When electrical coils are used as the generating device 32202, the electrical coils can be powered with a time varying magnetic field that induces a time varying electric field in a target pathway structure according to Faraday's law. An electromagnetic signal generated by the generating device 32202 can also be applied using electrochemical coupling, wherein electrodes are in direct contact with skin or another outer electrically conductive boundary of a target pathway structure. Yet in another embodiment according to the present invention, the electromagnetic signal generated by the generating device 32202 can also be applied using electrostatic coupling wherein an air gap exists between a generating device 32202 such as an electrode and a target pathway structure such as ions and ligands. An advantage of the present invention is that its ultra-lightweight coils and miniaturized circuitry allow for use with common physical therapy treatment modalities, and at any location for which tissue growth, pain relief, and tissue and organ healing is desired. An advantageous result of application of the present invention is that tissue growth, repair, and maintenance can be accomplished and enhanced anywhere and at any time. Yet another advantageous result of application of the present invention is that growth, repair, and maintenance of molecules, cells, tissues, and organs can be accomplished and enhanced anywhere and at any time. Another embodiment according to the present invention delivers PEMF for application to respiratory tissue that is infected with diseases such as sarcoidosis, granulomatous pneumonitis, pulmonary fibrosis, and “World Trade Center Cough.”

Induced time-varying currents from PEMF or PRF devices flow in a fibrous capsule formation and capsular contracture target pathway structure such as a molecule, cell, tissue, and organ, and it is these currents that are a stimulus to which cells and tissues can react in a physiologically meaningful manner The electrical properties of a fibrous capsule formation and capsular contracture target pathway structure affect levels and distributions of induced current. Molecules, cells, tissue, and organs are all in an induced current pathway such as cells in a gap junction contact. Ion or ligand interactions at binding sites on macromolecules that may reside on a membrane surface are voltage dependent chemical processes, that is electrochemical, that can respond to an induced electromagnetic field (“E”). Induced current arrives at these sites via a surrounding ionic medium. The presence of cells in a current pathway causes an induced current (“J”) to decay more rapidly with time (“J(t)”). This is due to an added electrical impedance of cells from membrane capacitance and ion binding time constants of binding and other voltage sensitive membrane processes such as membrane transport. Knowledge of ion binding time constants allows SNR to be evaluated for any EMF signal configuration. Preferably ion binding time constants in the range of about 1 to about 100 msec are used.

Another embodiment according to the present invention comprises known cellular responses to weak external stimuli such as heat, light, sound, ultrasound and electromagnetic fields. Cellular responses to such stimuli result in the production of protective proteins, for example, heat shock proteins, which enhance the ability of the cell, tissue, organ to withstand and respond to such external stimuli. Electromagnetic fields configured according to an embodiment of the present invention enhance the release of such compounds thus advantageously providing an improved means to enhance prophylactic protection and wellness of living organisms. After implant surgery there can be physiological deficiencies such as capsular contraction and excessive fibrous capsule formation states that can have a lasting and deleterious effect on an individual's well being and on the proper functioning of an implanted device. Those physiological deficiencies and states can be positively affected on a non-invasive basis by the therapeutic application of waveforms configured according to an embodiment of the present invention. In addition, electromagnetic waveforms configured according to an embodiment of the present invention can have a prophylactic effect on an implant area whereby formation of excessive fibrous tissue may be prevented.

One embodiment of the present invention relates to a therapeutically beneficial method of and apparatus for non-invasive pulsed electromagnetic treatment for enhanced condition, repair and growth of living tissue in animals, humans and plants. This beneficial method operates to selectively change the bioelectromagnetic environment associated with the cellular and tissue environment through the use of electromagnetic means such as PRF generators and applicator heads. More particularly use of electromagnetic means includes the provision of a flux path to a selectable body region, of a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter. Further, the repetition rate of such pulse bursts may vary from 0.01 to 10,000 Hz. Additionally a mathematically-definable parameter can be employed in lieu of said random amplitude envelope of the pulse bursts.

According to an embodiment of the present invention, by applying a random, or other high spectral density envelope, to a pulse burst envelope of mono-polar or bi-polar rectangular or sinusoidal pulses which induce peak electric fields between 10-8 and 100 millivolts per centimeter (mV/cm), a more efficient and greater effect can be achieved on biological healing processes applicable to both soft and hard tissues in humans, animals and plants. A pulse burst envelope of higher spectral density can advantageously and efficiently couple to physiologically relevant dielectric pathways, such as, cellular membrane receptors, ion binding to cellular enzymes, and general transmembrane potential changes thereby modulating angiogenesis and neovascularization.

Specifically, broad spectral density bursts of electromagnetic waveforms, configured to achieve maximum signal power within a bandpass of a biological target, are selectively applied to fibrous capsule formation and capsular contracture target pathway structures such as living organs, tissues, cells and molecules that are associated with excessive fibrous capsule formation and capsular contracture. Waveforms are selected using a novel amplitude/power comparison with that of thermal noise in a fibrous capsule formation and capsular contracture target pathway structure. Signals comprise bursts of at least one of sinusoidal, rectangular, chaotic and random wave shapes have frequency content in a range of 0.01 Hz to 100 MHz at 1 to 100,000 bursts per second, with a burst duration from 0.01 to 100 milliseconds, and a burst repetition rate from 0.01 to 1000 bursts/second. Peak signal amplitude at a fibrous capsule formation and capsular contracture target pathway structure such as tissue, lies in a range of 1 μV/cm to 100 mV/cm. Each signal burst envelope may be a random function providing a means to accommodate different electromagnetic characteristics of healing tissue. Preferably the present invention comprises a 20 millisecond pulse burst, repeating at 1 to 10 burst/second and comprising 0.5 to 200 microsecond symmetrical or asymmetrical pulses repeating at 10-5 to 100 kilohertz within the burst. The burst envelope can be modified 1/f function or any arbitrary function and can be applied at random repetition rates. Fixed repetition rates can also be used between about 0.1 Hz and about 1000 Hz. An induced electric field from about 10-8 mV/cm to about 100 mV/cm is generated. Another embodiment according to the present invention comprises a 4 millisecond of high frequency sinusoidal waves, such as 27.12 MHz, repeating at 1 to 100 bursts per second. An induced electric field from about 10-8 mV/cm to about 100 mV/cm is generated. Resulting waveforms can be delivered via inductive or capacitive coupling for 1 to 30 minute treatment sessions delivered according to predefined regimes by which PEMF treatment may be applied for 1 to 12 daily sessions, repeated daily. The treatment regimens for any waveform configured according to the instant invention may be fully automated. The number of daily treatments may be programmed to vary on a daily basis according to any predefined protocol.

Specifically, broad spectral density bursts of electromagnetic waveforms, configured to achieve maximum signal power within a bandpass of a biological target, are selectively applied to fibrous capsule formation and capsular contracture target pathway structures such as living organs, tissues, cells and molecules that are associated with excessive fibrous capsule formation and capsular contracture. Waveforms are selected using a novel amplitude/power comparison with that of thermal noise in a fibrous capsule formation and capsular contracture target pathway structure. Signals comprise bursts of at least one of sinusoidal, rectangular, chaotic and random wave shapes have frequency content in a range of 0.01 Hz to 100 MHz at 1 to 100,000 bursts per second, with a burst duration from 0.01 to 100 milliseconds, and a burst repetition rate from 0.01 to 1000 bursts/second. Peak signal amplitude at a fibrous capsule formation and capsular contracture target pathway structure such as tissue, lies in a range of 1 μV/cm to 100 mV/cm. Each signal burst envelope may be a random function providing a means to accommodate different electromagnetic characteristics of healing tissue. Preferably the present invention comprises a 20 millisecond pulse burst, repeating at 1 to 10 burst/second and comprising 0.5 to 200 microsecond symmetrical or asymmetrical pulses repeating at 10⁻⁵ to 100 kilohertz within the burst. The burst envelope can be modified 1/f function or any arbitrary function and can be applied at random repetition rates. Fixed repetition rates can also be used between about 0.1 Hz and about 1000 Hz. An induced electric field from about 10⁻⁸ mV/cm to about 100 mV/cm is generated. Another embodiment according to the present invention comprises a 4 millisecond of high frequency sinusoidal waves, such as 27.12 MHz, repeating at 1 to 100 bursts per second. An induced electric field from about 10⁻⁸ mV/cm to about 100 mV/cm is generated. Resulting waveforms can be delivered via inductive or capacitive coupling for 1 to 30 minute treatment sessions delivered according to predefined regimes by which PEMF treatment may be applied for 1 to 12 daily sessions, repeated daily. The treatment regimens for any waveform configured according to the instant invention may be fully automated. The number of daily treatments may be programmed to vary on a daily basis according to any predefined protocol.

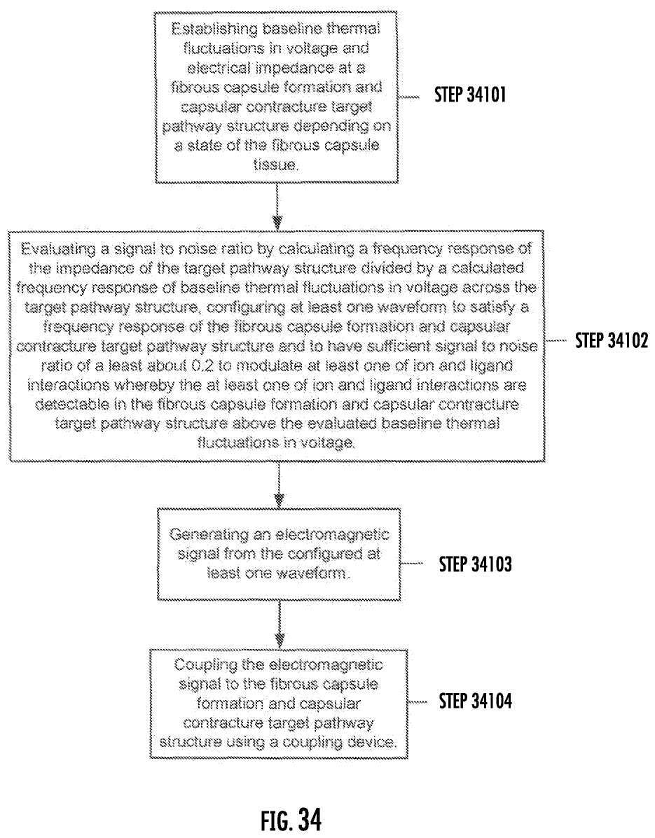

Referring to FIG. 34 wherein FIG. 34 is a flow diagram of a method for generating electromagnetic signals to be coupled to a fibrous capsule formation and capsular contracture target pathway structure according to an embodiment of the present invention, a fibrous capsule formation and capsular contracture target pathway structure such as ions and ligands, is identified. Establishing a baseline background activity such as baseline thermal fluctuations in voltage and electrical impedance, at the fibrous capsule formation and capsular contracture target pathway structure by determining a state of at least one of a cell and a tissue at the fibrous capsule formation and capsular contracture target pathway structure, wherein the state is at least one of resting, growing, replacing, and responding to injury. (STEP 34101) The state of the at least one of a cell and a tissue is determined by its response to injury or insult. Configuring at least one waveform to have sufficient signal to noise ratio to modulate at least one of ion and ligand interactions whereby the at least one of ion and ligand interactions are detectable in the fibrous capsule formation and capsular contracture target pathway structure above the established baseline thermal fluctuations in voltage and electrical impedance. The EMF signal can be generated by using at least one waveform configured by applying a mathematical model such as an equation, formula, or function having at least one waveform parameter that satisfies an SNR or Power SNR mathematical model of at least about 0.2, to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure (STEP 34102). Repetitively generating an electromagnetic signal from the configured at least one waveform (STEP 34103). Coupling the electromagnetic signal to the fibrous capsule formation and capsular contracture target pathway structure using a coupling device (STEP 34104). The generated electromagnetic signals can be coupled for therapeutic and prophylactic purposes. The coupling enhances a stimulus that cells and tissues react to in a physiological meaningful manner for example, an increase in angiogenesis, neovascularization and vascularogenesis or other physiological effects related to the improvement of excessive fibrous tissue or capsular contracture. Application of electromagnetic signals using an embodiment according to the present invention is extremely safe and efficient since the application of electromagnetic signals configured according to the present invention is non-invasive and athermal.

In the present invention, a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model. A repetitive electromagnetic signal can be generated for example inductively or capacitively, from the configured at least one waveform. The electromagnetic signal is coupled to a fibrous capsule formation and capsular contracture target pathway structure such as ions and ligands by output of a coupling device such as an electrode or an inductor, placed in close proximity to the fibrous capsule formation and capsular contracture target pathway structure using a positioning device. The coupling enhances modulation of binding of ions and ligands to regulatory molecules, tissues, cells, and organs. According to an embodiment of the present invention EMF signals configured using SNR analysis to match the bandpass of a second messenger whereby the EMF signals can act as a first messenger to modulate biochemical cascades such as production of cytokines, Nitric Oxide, Nitric Oxide Synthase and growth factors that are related to tissue growth and repair. A detectable E field amplitude is produced within a frequency response of Ca²⁺ binding.

FIG. 32 illustrates an embodiment of an apparatus according to the present invention. The apparatus is constructed to be self-contained, lightweight, and portable. A miniature control circuit 33201 is connected to a generating device such as an electrical coil 33202. The miniature control circuit 33201 is constructed in a manner that applies a mathematical model that is used to configure waveforms. The configured waveforms have to satisfy a Power SNR model so that for a given and known fibrous capsule formation and capsular contracture target pathway structure, it is possible to choose waveform parameters that satisfy a frequency response of the fibrous capsule formation and capsular contracture target pathway structure and Power SNR of at least about 0.2 to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure. An embodiment according to the present invention applies a mathematical model to induce a time-varying magnetic field and a time-varying electric field in a fibrous capsule formation and capsular contracture target pathway structure such as ions and ligands, comprising about 0.001 to about 100 msec bursts of about 1 to about 100 microsecond rectangular pulses, having a burst duration of about 0.01 to 100,000 microseconds and repeating at about 0.1 to about 100 pulses per second. Peak amplitude of the induced electric field is between about 1 uV/cm and about 100 mV/cm, that can be constant or varied according to a mathematical function, for example a modified 1/f function where f=frequency. A waveform having the parameters described earlier in connection with FIG. 5 may be applied to a fibrous capsule formation and capsular contracture target pathway structure such as ions and ligands, preferably for a total exposure time of under 1 minute to 240 minutes daily. However other exposure times can be used. Waveforms configured by the miniature control circuit 33201 are directed to a generating device 33202 such as electrical coils. Preferably, the generating device 33202 is a conformable coil for example pliable, comprising one or more turns of electrically conducting wire in a generally circular or oval shape however other shapes can be used. The generating device 33202 delivers a pulsing magnetic field configured according to a mathematical model that can be used to provide treatment to a fibrous capsule formation and capsular contracture target pathway structure such as mammary tissue. The miniature control circuit applies a pulsing magnetic field for a prescribed time and can automatically repeat applying the pulsing magnetic field for as many applications as are needed in a given time period, for example 12 times a day. The miniature control circuit can be configured to be programmable applying pulsing magnetic fields for any time repetition sequence. An embodiment according to the present invention can be positioned to treat fibrous capsule tissue by being incorporated with a positioning device such as a bandage, a vest, a brassiere, or an anatomical support thereby making the unit self-contained. Coupling a pulsing magnetic field to a fibrous capsule formation and capsular contracture target pathway structure such as ions and ligands, therapeutically and prophylactically reduces inflammation thereby reducing pain and promotes healing in treatment areas. When electrical coils are used as the generating device 33202, the electrical coils can be powered with a time varying magnetic field that induces a time varying electric field in a fibrous capsule formation and capsular contracture target pathway structure according to Faraday's law. An electromagnetic signal generated by the generating device 33202 can also be applied using electrochemical coupling, wherein electrodes are in direct contact with skin or another outer electrically conductive boundary of a fibrous capsule formation and capsular contracture target pathway structure. Yet in another embodiment according to the present invention, the electromagnetic signal generated by the generating device 33202 can also be applied using electrostatic coupling wherein an air gap exists between a generating device 35202 such as an electrode and a fibrous capsule formation and capsular contracture target pathway structure such as ions and ligands. An advantage of the present invention is that its ultra-lightweight coils and miniaturized circuitry allow for use with common physical therapy treatment modalities, and at any location for which tissue growth, pain relief, and tissue and organ healing is desired. An advantageous result of application of the present invention is that tissue growth, repair, and maintenance can be accomplished and enhanced anywhere and at any time. Yet another advantageous result of application of the present invention is that growth, repair, and maintenance of molecules, cells, tissues, and organs can be accomplished and enhanced anywhere and at any time. Another embodiment according to the present invention delivers PEMF for application to capsular contracture and excessive fibrous capsule tissue that resulted from implant surgery such as breast augmentation.

The miniature control circuit 33201 may be similar or identical to the miniature control circuit 300 shown in FIG. 3. The miniature control circuit 300 produces waveforms that drive a generating device such as wire coils described above in FIG. 32. The miniature control circuit can be activated by any activation means such as an on/off switch. The miniature control circuit 300 has a power source such as a lithium battery 301. Preferably the power source has an output voltage of 3.3 V but other voltages can be used. In another embodiment according to the present invention the power source can be an external power source such as an electric current outlet such as an AC/DC outlet, coupled to the present invention for example by a plug and wire. A switching power supply 302 controls voltage to a micro-controller 303. Preferably the micro-controller 303 uses an 8 bit 4 MHz micro-controller 303 but other bit MHz combination micro-controllers may be used. The switching power supply 302 also delivers current to storage capacitors 304. Preferably the storage capacitors 304 having a 220 μF output but other outputs can be used. The storage capacitors 304 allow high frequency pulses to be delivered to a coupling device such as inductors (Not Shown). The micro-controller 303 also controls a pulse shaper 305 and a pulse phase timing control 306. The pulse shaper 305 and pulse phase timing control 306 determine pulse shape, burst width, burst envelope shape, and burst repetition rate. In an embodiment according to the present invention the pulse shaper 305 and phase timing control 306 are configured such that the waveforms configured are detectable above background activity at a fibrous capsule formation and capsular contracture target pathway structure by satisfying at least one of a SNR and Power SNR mathematical model. An integral waveform generator, such as a sine wave or arbitrary number generator can also be incorporated to provide specific waveforms. A voltage level conversion sub-circuit 307 controls an induced field delivered to a fibrous capsule formation and capsular contracture target pathway structure. A switching Hexfet 308 allows pulses of randomized amplitude to be delivered to output 72309 that routes a waveform to at least one coupling device such as an inductor. The micro-controller 303 can also control total exposure time of a single treatment of a fibrous capsule formation and capsular contracture target pathway structure such as a molecule, cell, tissue, and organ. The miniature control circuit 300 can be constructed to be programmable and apply a pulsing magnetic field for a prescribed time and to automatically repeat applying the pulsing magnetic field for as many applications as are needed in a given time period, for example 10 times a day. In a preferred embodiment, the pulsing magnetic field comprises waveforms having the same parameters as those described previously in connection with FIG. 5. In addition, treatment times of about 1 minutes to about 30 minutes are preferred.

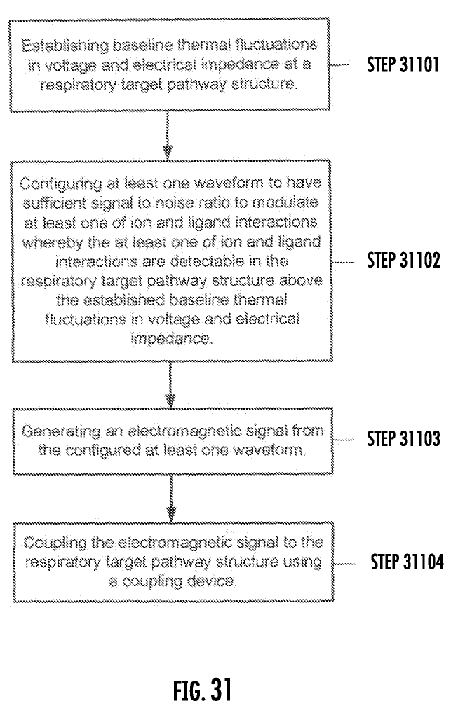

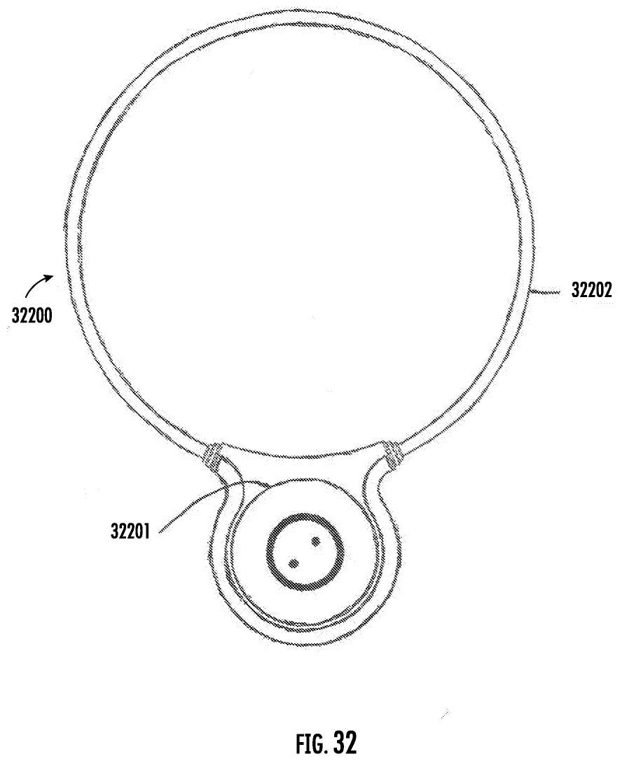

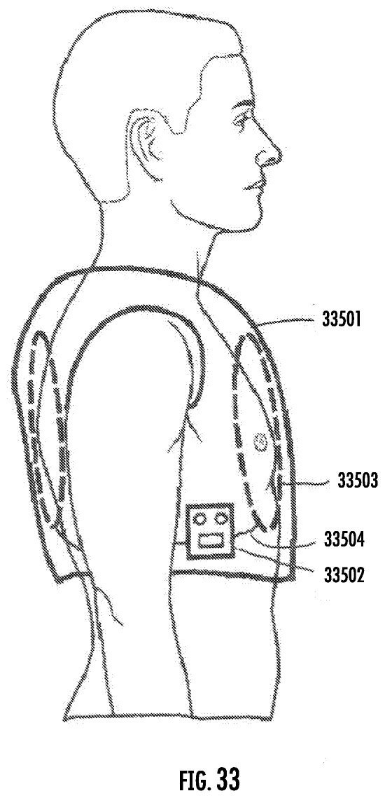

FIG. 33 illustrates an embodiment of an apparatus according to the present invention. A garment 33501 such as a vest is constructed out of materials that are lightweight and portable such as nylon but other materials can be used. A miniature control circuit 33502 is coupled to a generating device such as an electrical coil 33503. Preferably the miniature control circuit 33502 and the electrical coil 33503 are constructed in a manner as described above in reference to FIG. 64. The miniature control circuit and the electrical coil can be connected with a connecting means such as a wire 33504. The connection can also be direct or wireless. The electrical coil 33503 is integrated into the garment 33501 such that when a user wears the garment 33501, the electrical coil is positioned near a lung or both lungs of the user. An advantage of the present invention is that its ultra-lightweight coils and miniaturized circuitry allow for the garment 33501 to be completely self-contained, portable, and lightweight. An additionally advantageous result of the present invention is that the garment 33501 can be constructed to be inconspicuous when worn and can be worn as an outer garment such as a shirt or under other garments, so that only the user will know that the garment 33501 is being worn and treatment is being applied. Use with common physical therapy treatment modalities, and at any respiratory location for which tissue growth, pain relief, and tissue and organ healing is easily obtained. An advantageous result of application of the present invention is that tissue growth, repair, and maintenance can be accomplished and enhanced anywhere and at any time. Yet another advantageous result of application of the present invention is that growth, repair, and maintenance of molecules, cells, tissues, and organs can be accomplished and enhanced anywhere and at any time. Another embodiment according to the present invention delivers PEMF for application to respiratory tissue that is infected with diseases such as sarcoidosis, granulomatous pneumonitis, pulmonary fibrosis, and “World Trade Center Cough.”

Another embodiment according to the present invention comprises known cellular responses to weak external stimuli such as heat, light, sound, ultrasound and electromagnetic fields. Cellular responses to such stimuli result in the production of protective proteins, for example, heat shock proteins, which enhance the ability of the cell, tissue, organ to withstand and respond to such external stimuli. Electromagnetic fields configured according to an embodiment of the present invention enhance the release of such compounds thus advantageously providing an improved means to enhance prophylactic protection and wellness of living organisms. After implant surgery there can be physiological deficiencies such as capsular contraction and excessive fibrous capsule formation states that can have a lasting and deleterious effect on an individual's well being and on the proper functioning of an implanted device. Those physiological deficiencies and states can be positively affected on a non-invasive basis by the therapeutic application of waveforms configured according to an embodiment of the present invention. In addition, electromagnetic waveforms configured according to an embodiment of the present invention can have a prophylactic effect on an implant area whereby formation of excessive fibrous tissue may be prevented.

Specifically, broad spectral density bursts of electromagnetic waveforms, configured to achieve maximum signal power within a bandpass of a biological target, are selectively applied to fibrous capsule formation and capsular contracture target pathway structures such as living organs, tissues, cells and molecules that are associated with excessive fibrous capsule formation and capsular contracture. Waveforms are selected using a novel amplitude/power comparison with that of thermal noise in a fibrous capsule formation and capsular contracture target pathway structure. Signals comprise bursts of at least one of sinusoidal, rectangular, chaotic and random wave shapes have frequency content in a range of 0.01 Hz to 100 MHz at 1 to 100,000 bursts per second, with a burst duration from 0.01 to 100 milliseconds, and a burst repetition rate from 0.01 to 1000 bursts/second. Peak signal amplitude at a fibrous capsule formation and capsular contracture target pathway structure such as tissue, lies in a range of 1 μV/cm to 100 mV/cm. Each signal burst envelope may be a random function providing a means to accommodate different electromagnetic characteristics of healing tissue. Preferably the present invention comprises a 20 millisecond pulse burst, repeating at 1 to 10 burst/second and comprising 0.5 to 200 microsecond symmetrical or asymmetrical pulses repeating at 10-5 to 100 kilohertz within the burst. The burst envelope can be modified 1/f function or any arbitrary function and can be applied at random repetition rates. Fixed repetition rates can also be used between about 0.1 Hz and about 1000 Hz. An induced electric field from about 10-8 mV/cm to about 100 mV/cm is generated. Another embodiment according to the present invention comprises a 4 millisecond of high frequency sinusoidal waves, such as 27.12 MHz, repeating at 1 to 100 bursts per second. An induced electric field from about 10-8 mV/cm to about 100 mV/cm is generated. Resulting waveforms can be delivered via inductive or capacitive coupling for 1 to 30 minute treatment sessions delivered according to predefined regimes by which PEMF treatment may be applied for 1 to 12 daily sessions, repeated daily. The treatment regimens for any waveform configured according to the instant invention may be fully automated. The number of daily treatments may be programmed to vary on a daily basis according to any predefined protocol.

Referring to FIG. 34 wherein FIG. 34 is a flow diagram of a method for generating electromagnetic signals to be coupled to a fibrous capsule formation and capsular contracture target pathway structure according to an embodiment of the present invention, a fibrous capsule formation and capsular contracture target pathway structure such as ions and ligands, is identified. Establishing a baseline background activity such as baseline thermal fluctuations in voltage and electrical impedance, at the fibrous capsule formation and capsular contracture target pathway structure by determining a state of at least one of a cell and a tissue at the fibrous capsule formation and capsular contracture target pathway structure, wherein the state is at least one of resting, growing, replacing, and responding to injury. (STEP 34101) The state of the at least one of a cell and a tissue is determined by its response to injury or insult. Configuring at least one waveform to have sufficient signal to noise ratio to modulate at least one of ion and ligand interactions whereby the at least one of ion and ligand interactions are detectable in the fibrous capsule formation and capsular contracture target pathway structure above the established baseline thermal fluctuations in voltage and electrical impedance. The EMF signal can be generated by using at least one waveform configured by applying a mathematical model such as an equation, formula, or function having at least one waveform parameter that satisfies an SNR or Power SNR mathematical model of at least about 0.2, to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure (STEP 34102). Repetitively generating an electromagnetic signal from the configured at least one waveform (STEP 34103). Coupling the electromagnetic signal to the fibrous capsule formation and capsular contracture target pathway structure using a coupling device (STEP 34104). The generated electromagnetic signals can be coupled for therapeutic and prophylactic purposes. The coupling enhances a stimulus that cells and tissues react to in a physiological meaningful manner for example, an increase in angiogenesis, neovascularization and vascularogenesis or other physiological effects related to the improvement of excessive fibrous tissue or capsular contracture. Application of electromagnetic signals using an embodiment according to the present invention is extremely safe and efficient since the application of electromagnetic signals configured according to the present invention is non-invasive and athermal.

In the present invention, a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model. A repetitive electromagnetic signal can be generated for example inductively or capacitively, from the configured at least one waveform. The electromagnetic signal is coupled to a fibrous capsule formation and capsular contracture target pathway structure such as ions and ligands by output of a coupling device such as an electrode or an inductor, placed in close proximity to the fibrous capsule formation and capsular contracture target pathway structure using a positioning device. The coupling enhances modulation of binding of ions and ligands to regulatory molecules, tissues, cells, and organs. According to an embodiment of the present invention EMF signals configured using SNR analysis to match the bandpass of a second messenger whereby the EMF signals can act as a first messenger to modulate biochemical cascades such as production of cytokines, Nitric Oxide, Nitric Oxide Synthase and growth factors that are related to tissue growth and repair. A detectable E field amplitude is produced within a frequency response of Ca2+ binding.

An apparatus described in connection with FIG. 32 may be used to treat fibrous capsule formation and capsular contraction. The apparatus is constructed to be self-contained, lightweight, and portable. A miniature control circuit 32201 is connected to a generating device such as an electrical coil 32202. The miniature control circuit 32201 is constructed in a manner that applies a mathematical model that is used to configure waveforms. The configured waveforms have to satisfy a Power SNR model so that for a given and known fibrous capsule formation and capsular contracture target pathway structure, it is possible to choose waveform parameters that satisfy a frequency response of the fibrous capsule formation and capsular contracture target pathway structure and Power SNR of at least about 0.2 to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure. An embodiment according to the present invention applies a mathematical model to induce a time-varying magnetic field and a time-varying electric field in a fibrous capsule formation and capsular contracture target pathway structure such as ions and ligands, comprising about 0.001 to about 100 msec bursts of about 1 to about 100 microsecond rectangular pulses, having a burst duration of about 0.01 to 100,000 microseconds and repeating at about 0.1 to about 100 pulses per second. Peak amplitude of the induced electric field is between about 1 uV/cm and about 100 mV/cm, that can be constant or varied according to a mathematical function, for example a modified 1/f function where f=frequency. A waveform configured using an embodiment according to the present invention may be applied to a fibrous capsule formation and capsular contracture target pathway structure such as ions and ligands, preferably for a total exposure time of under 1 minute to 240 minutes daily. However other exposure times can be used. Waveforms configured by the miniature control circuit 32201 are directed to a generating device 32202 such as electrical coils. Preferably, the generating device 32202 is a conformable coil for example pliable, comprising one or more turns of electrically conducting wire in a generally circular or oval shape however other shapes can be used. The generating device 32202 delivers a pulsing magnetic field configured according to a mathematical model that can be used to provide treatment to a fibrous capsule formation and capsular contracture target pathway structure such as mammary tissue. The miniature control circuit applies a pulsing magnetic field for a prescribed time and can automatically repeat applying the pulsing magnetic field for as many applications as are needed in a given time period, for example 12 times a day. The miniature control circuit can be configured to be programmable applying pulsing magnetic fields for any time repetition sequence. An embodiment according to the present invention can be positioned to treat fibrous capsule tissue by being incorporated with a positioning device such as a bandage, a vest, a brassiere, or an anatomical support thereby making the unit self-contained. Coupling a pulsing magnetic field to a fibrous capsule formation and capsular contracture target pathway structure such as ions and ligands, therapeutically and prophylactically reduces inflammation thereby reducing pain and promotes healing in treatment areas. When electrical coils are used as the generating device 32202, the electrical coils can be powered with a time varying magnetic field that induces a time varying electric field in a fibrous capsule formation and capsular contracture target pathway structure according to Faraday's law. An electromagnetic signal generated by the generating device 32202 can also be applied using electrochemical coupling, wherein electrodes are in direct contact with skin or another outer electrically conductive boundary of a fibrous capsule formation and capsular contracture target pathway structure. Yet in another embodiment according to the present invention, the electromagnetic signal generated by the generating device 32202 can also be applied using electrostatic coupling wherein an air gap exists between a generating device 32202 such as an electrode and a fibrous capsule formation and capsular contracture target pathway structure such as ions and ligands. An advantage of the present invention is that its ultra-lightweight coils and miniaturized circuitry allow for use with common physical therapy treatment modalities, and at any location for which tissue growth, pain relief, and tissue and organ healing is desired. An advantageous result of application of the present invention is that tissue growth, repair, and maintenance can be accomplished and enhanced anywhere and at anytime. Yet another advantageous result of application of the present invention is that growth, repair, and maintenance of molecules, cells, tissues, and organs can be accomplished and enhanced anywhere and at anytime. Another embodiment according to the present invention delivers PEMF for application to capsular contracture and excessive fibrous capsule tissue that resulted from implant surgery such as breast augmentation.

The miniature control circuit 32201 may be similar or identical in structure to miniature control circuit 300 shown in FIG. 3. The miniature control circuit 300 produces waveforms that drive a generating device such as wire coils described above in FIG. 32. The miniature control circuit can be activated by any activation means such as an on/off switch. The miniature control circuit 300 has a power source such as a lithium battery 301. Preferably the power source has an output voltage of 3.3 V but other voltages can be used. In another embodiment according to the present invention the power source can be an external power source such as an electric current outlet such as an AC/DC outlet, coupled to the present invention for example by a plug and wire. A switching power supply 302 controls voltage to a micro-controller 303. Preferably the micro-controller 303 uses an 8 bit 4 MHz micro-controller 72303 but other bit MHz combination micro-controllers may be used. The switching power supply 302 also delivers current to storage capacitors 304. Preferably the storage capacitors 304 having a 220 uF output but other outputs can be used. The storage capacitors 304 allow high frequency pulses to be delivered to a coupling device such as inductors (Not Shown). The micro-controller 303 also controls a pulse shaper 305 and a pulse phase timing control 306. The pulse shaper 305 and pulse phase timing control 306 determine pulse shape, burst width, burst envelope shape, and burst repetition rate. In an embodiment according to the present invention the pulse shaper 305 and phase timing control 306 are configured such that the waveforms configured are detectable above background activity at a fibrous capsule formation and capsular contracture target pathway structure by satisfying at least one of a SNR and Power SNR mathematical model. An integral waveform generator, such as a sine wave or arbitrary number generator can also be incorporated to provide specific waveforms. A voltage level conversion sub-circuit 307 controls an induced field delivered to a fibrous capsule formation and capsular contracture target pathway structure.

A switching Hexfet 308 allows pulses of randomized amplitude to be delivered to output 309 that routes a waveform to at least one coupling device such as an inductor. The micro-controller 303 can also control total exposure time of a single treatment of a fibrous capsule formation and capsular contracture target pathway structure such as a molecule, cell, tissue, and organ. The miniature control circuit 300 can be constructed to be programmable and apply a pulsing magnetic field for a prescribed time and to automatically repeat applying the pulsing magnetic field for as many applications as are needed in a given time period, for example 10 times a day. In a preferred embodiment, the pulsing magnetic field comprises waveforms having the same parameters as those described previously in connection with FIG. 5. In addition, treatment times of about 1 minutes to about 30 minutes are preferred.

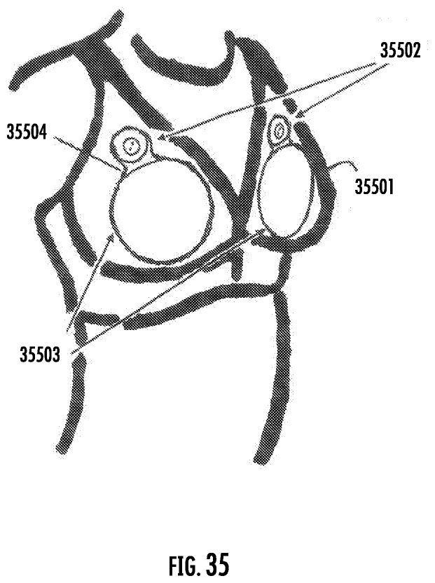

FIG. 35 illustrates an embodiment of an apparatus according to the present invention. A garment 35501 such as a brassiere is constructed out of materials that are lightweight and portable such as nylon but other materials can be used. A miniature control circuit 35502 is coupled to a generating device such as an electrical coil 35503. Preferably the miniature control circuit 35502 and the electrical coil 35503 are constructed in a manner as described above in reference to FIG. 32. The miniature control circuit and the electrical coil can be connected with a connecting means such as a wire 35504. The connection can also be direct or wireless. The electrical coil 35503 is integrated into the garment 35501 such that when a user wears the garment 35501, the electrical coil is positioned near an excessive fibrous capsule formation location or capsular contracture location of the user. An advantage of the present invention is that its ultra-lightweight coils and miniaturized circuitry allow for the garment 35501 to be completely self-contained, portable, and lightweight. An additionally advantageous result of the present invention is that the garment 3501 can be constructed to be inconspicuous when worn and can be worn as an outer garment such as a shirt or under other garments, so that only the user will know that the garment 35501 is being worn and treatment is being applied. Use with common physical therapy treatment modalities, and at any excessive fibrous capsule location or capsular contracture location for which pain relief, and tissue and organ healing is easily obtained. An advantageous result of application of the present invention is that tissue growth, repair, and maintenance can be accomplished and enhanced anywhere and at anytime. Yet another advantageous result of application of the present invention is that growth, repair, and maintenance of molecules, cells, tissues, and organs can be accomplished and enhanced anywhere and at any time. Another embodiment according to the present invention delivers PEMF for application to fibrous capsules.

### EXAMPLE 9

In this example six patients who had developed capsular contracture after receiving bilateral breast implants were treated with a special support brassiere having embedded coils located in each cup and a generator for each coil located in a special pocket in the strap above each cup as described in FIG. 35 above. PEMF signals generated by the apparatus configured according to an embodiment of the present invention comprised a repetitive burst of radio frequency sinusoidal waves configured according to an embodiment of the present invention. The PEMF signal induced a peak electric field in a range of 1 to 10 mV/cm. All patients were provided a regimen that comprised six thirty minute sessions for days 1 to 3 post implant, four sessions for days 4 to 6 post implant, and two sessions for all subsequent days. Clinical evaluation demonstrated that by day 7 the fibrous capsule was significantly softer and patients reported significantly less pain and discomfort than prior to the treatment. Clinical evaluations at one and three months post PEMF treatment revealed significant resolution of the fibrous capsule and its corresponding symptoms.

Pulsed electromagnetic fields (PEMF) reduce postoperative pain and narcotic requirements in breast augmentation, reduction, and reconstruction patients. PEMF treatment can also be used to reduce post-operative pain in other surgical procedures. PEMF enhances calmodulin-dependent nitric oxide, which enhances cyclic guanosine monophosphate signaling and phosphodiesterase activity, which blocks cyclic guanosine monophosphate. The following embodiment of the invention describes means to configure PEMF dosing to minimize the effect of the competing response of phosphodiesterace activity.

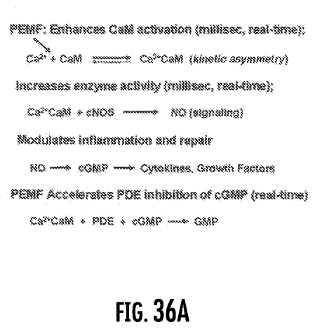

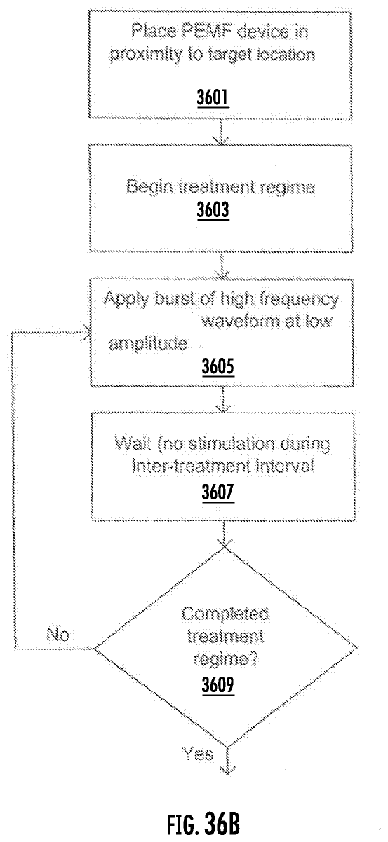

FIG. 36B is a flow diagram of a method for treating a subject with a PEMF. In some variations, before beginning the treatment, one or more (or a range of) waveforms may be determined that target the appropriate pathway for the target tissue. In such embodiments, once this determination is made, electromagnetic fields are applied to the target location.

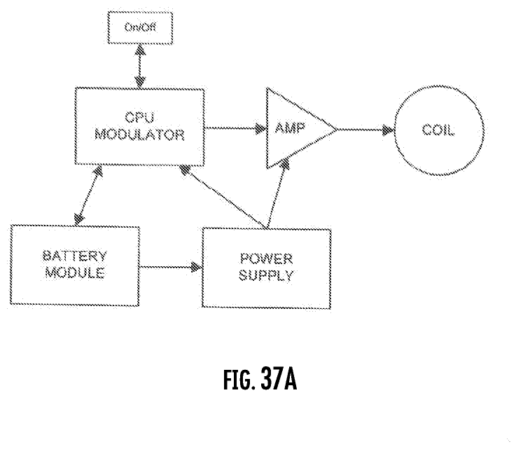

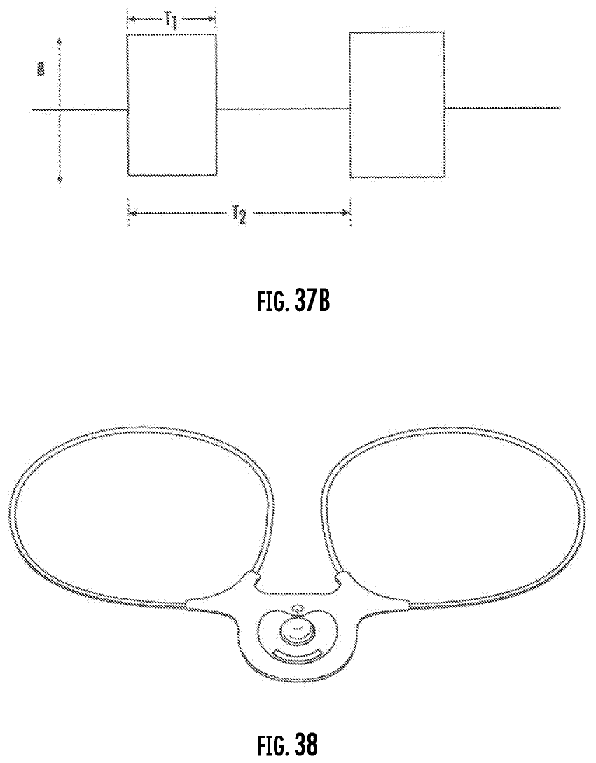

FIG. 37A illustrates a block diagram of an EMF delivery apparatus as described according to some embodiments. As shown in FIG. 37AA, the apparatus may have miniaturized circuitry for use with a coil applicator. In some embodiments, the apparatus may include a CPU MODULATOR, a BATTERY MODULE, a POWER SUPPLY, On/Off switch, and an output amplifier, AMP, as illustrated. In further variations, the CPU MODULATOR may be an 8 bit 4 MHz micro-controller; however, other suitable bit-MHz combination micro-controllers may be used as well. For example, in some embodiments, the CPU MODULATOR may be programmed for a given carrier frequency or pulse duration, such as about 27.12 MHz sinusoidal wave. Moreover, the CPU MODULATOR may be programmed for a given burst duration, for example about 3 msec. In further variations, the CPU MODULATOR may be programmed to provide a given in situ peak electric field, for example 20 V/m; or a given treatment time, for example about 15 minutes; and/or a given treatment regimen, for example about 10 minutes about every hour. The CPU MODULATOR may also be programmed to deliver an EMF waveform to the target ion binding pathway.

Some embodiments combine the signal generation and coil or electrode applicator into one portable or disposable unit such as that illustrated in FIG. 32 for the case of an inductively coupled signal. In some variations, when electrical coils are used as the applicator, the electrical coils can be powered with a time varying magnetic field that induces a time varying electric field in a target pathway structure according to Faraday's law. An electromagnetic field generated by a circuit such as shown in FIG. 37A can also be applied using electrochemical coupling, wherein electrodes are in direct contact with skin or another outer electrochemically conductive boundary of a target pathway structure.

In yet another embodiment, the electromagnetic field generated by the generating circuit of FIG. 37A can also be applied using electrostatic coupling wherein an air gap exists between a generating device such as an electrode and a target pathway structure such as a molecule, cell, tissue, and organ of a plant animal or human Advantageously, the ultra-lightweight coils and miniaturized circuitry, according to some embodiments, allow for use with common physical therapy treatment modalities and at any location on a plant, animal or human for which any therapeutic or prophylactic effect is desired. An advantageous result of application of some embodiments described is that a living organism's wellbeing can be maintained and enhanced.

Referring to FIG. 37B, an embodiment according to the present invention of an induced electric field waveform delivered to a target pathway structure is illustrated. As shown in FIG. 37B, burst duration and period are represented by T1 and T2, respectively. In some embodiments, the signal within the rectangular box designated at T1 can be, rectangular, sinusoidal, chaotic or random, provided that the waveform duration or carrier period is less than one-half of the target ion bound time. The peak induced electric field is related to the peak induced magnetic field, shown as B in FIG. 37B, via Faraday's Law of Induction.

In one embodiment, the method shown in FIG. 36B may be performed using a self-contained, lightweight, and portable apparatus of the type shown in FIG. 32. A circuit control/signal generator 32201 may be held within a (optionally wearable) housing and connected to a generating member such as an electrical coil 32202. In some embodiments, the circuit control/signal generator 32201 is constructed in a manner that given a target pathway within a target tissue, it is possible to choose waveform parameters that satisfy a frequency response of the target pathway within the target tissue. For some embodiments, circuit control/signal generator 32201 applies mathematical models or results of such models that approximate the kinetics of ion binding in biochemical pathways. Waveforms configured by the circuit control/signal generator 32201 are directed to a generating member 32202. In some variations, the generating member 32202 comprises electrical coils that are pliable and comfortable. In further embodiments, the generating member 32202 is made from one or more turns of electrically conducting wire in a generally circular or oval shape, any other suitable shape. In further variations, the electrical coil is a circular wire applicator with a diameter that allows encircling of a subject's cranium. In some embodiments, the diameter is between approximately 6-8 inches. In general, the size of the coil may be fixed or adjustable and the circuit control/signal generator may be matched to the material and the size of the applicator to provide the desired treatment.

The apparatus 32200 may deliver a pulsing magnetic field that can be used to provide treatment. In some embodiments, the device 32200 may apply a pulsing magnetic field for a prescribed time and can automatically repeat applying the pulsing magnetic field for as many applications as are needed in a given time period, e.g. 6-12 times a day. The device 32200 can be configured to apply pulsing magnetic fields for any time repetition sequence. Without being bound to any theory, it is believed that when electrical coils are used as a generating member 32202, the electrical coils can be powered with a time varying magnetic field that induces a biologically and therapeutically effective time varying electric field in a target tissue location.

In other embodiments, an electromagnetic field generated by the generating member 32202 can be applied using electrochemical coupling, wherein electrodes are in direct contact with skin or another outer electrically conductive boundary of the target tissue (e.g. skull or scalp). In other variations, the electromagnetic field generated by the generating member 32202 can also be applied using electrostatic coupling wherein an air gap exists between a generating member 32202 such as an electrode and the target tissue. In further examples, a signal generator and battery are housed in the miniature circuit control/signal generator 32201 and the miniature circuit control/signal generator 32201 may contain an on/off switch and light indicator. In further embodiments, the activation and control of the treatment device may be done via remote control such as by way of a fob that may be programmed to interact with a specific individual device. In other variations, the treatment device further includes a history feature that records the treatment parameters carried out by the device such that the information is recorded in the device itself and/or can be transmitted to another device such as computer, smart phone, printer, or other medical equipment/device.

In other variations, the treatment device 32200 has adjustable dimensions to accommodate fit to a variety of patient sizes and anatomy. For example, the generating member 23202 may comprise modular components which can be added or removed by mated attaching members. Alternatively, the treatment device 32200 may contain a detachable generating member (e.g. detachable circular coil or other configurations) that can be removed and replaced with configurations that are better suited for the particular patient's needs. A circular coil generating member 32202 may be removed and replaced with an elongate generating member such that PEMF treatment can be applied where other medical equipment may obstruct access by a circular generating member 32202. In other variations, the generating member may be made from Litz wire that allows the generating member to flex and fold to accommodate different target areas or sizes.

The PEMF devices disclosed herein can be used to treat patients with post-operative pain. Acute postoperative pain is a significant medical problem. Postoperative pain must be managed effectively to optimize surgical outcomes, reduce morbidity, shorten the duration of hospital stay, and control ever-increasing health-care costs [1]. For the vast majority of surgical procedures, pain mechanisms involve increased sensitivity of nociceptors due to increased presence of proinflammatory cytokines in the wound milieu [2]. Narcotics are most commonly used to treat postoperative pain; however, narcotics do not reduce nociceptor sensitivity and cause undesirable side effects and potential addiction. Alternative approaches to decrease post-operative pain involve slowing the appearance of proinflammatory agents at the surgical site [2].

To this end, a new modality, nonthermal, nonpharmacologic radio frequency pulsed electromagnetic field (PEMF) therapy has been reported to instantaneously enhance calmodulin (CaM)-dependent nitric oxide (NO) release in challenged cells and tissues. This, in turn, enhances the body's primary anti-inflammatory pathway, CaM-dependent nitric oxide/cyclic guanosine monophosphate (NO/cGMP) signaling [3e7]. In the surgical context, NO/cGMP signaling decreases the rate of release of proinflammatory cytokines, such as interleukin-1 beta (IL [interleukin]-1b) [8], and increases the release of growth factors, such as fibroblast growth factor-2 (FGF-2) [9], in the wound milieu. This mechanism is schematically represented in FIG. 36A. PEMF modulation of angiogenesis via effects on FGF-2 has been reported [10-15]. In some studies, the PEMF effect could be blocked with an FGF-2 inhibitor, consistent with a PEMF effect on NO/cGMP signaling [12, 13].

In the clinical setting, PEMF has been reported to accelerate postoperative pain decrease, with a concomitant reduction in narcotic requirements, in double-blinded, randomized clinical studies on breast reduction (BR) [16], breast augmentation [17, 18], and autologous flap breast reconstruction [19]. The BR study also showed that PEMF reduced inflammation by reducing IL-1 beta more than two-fold in the wound exudate, which correlated with the higher rate of pain reduction from PEMF [16]. PEMF can and has been used throughout the body, including after abdominoplasties, major intra-abdominal surgery, extremity procedures, and facial fat grafting [20, 21].

Taken together, preclinical and clinical results support an anti-inflammatory mechanism for PEMF based on modulation of CaM-dependent NO/cGMP signaling. However, the NO/cGMP cascade is dynamic [22] and regulated, in part, by phosphodiesterase (PDE) inhibition of cyclic guanosine monophosphate (cGMP) [23]. This inhibition is particularly important for PEMF therapy because PDE isoenzymes are also CaM-dependent, meaning the timing of PDE activity is modulated by the same PEMF signal that modulates the timing of NO/cGMP signaling [24]. Thus, although the dynamics of NO/cGMP signaling in challenged tissue can be modulated by PEMF, the effect of PEMF dosing on the competing dynamics of CaM-dependent NO/cGMP signaling and PDE inhibition of cGMP on pain outcome must be taken into account.

Example 10

A number of patients with post-operative pain were treated with PEMF and studied. Specifically, two prospective, nonrandomized, active cohorts of breast reduction patients, with 15 min PEMF per 2 h; “Q2 (active)”, and 5 min PEMF per 20 min; “5/20 (active)”, dosing regimens were added to a double-blind clinical study wherein 20 min PEMF per 4 h, “Q4 (active)”, dosing was shown to significantly accelerate postoperative pain reduction compared with Q4 shams. Postoperative visual analog scale pain scores and narcotic use were compared.

Data from 50 patients were available for analysis. The change in VAS scores normalized to 1 h for each cohort is summarized in FIG. 39. The rate of postoperative pain decrease in the first 24 h postoperative for patients in the Q4 (active) and Q2 (active) cohorts was not significantly different (P=0.485), but was nearly 3-fold faster than that for patients in the 5/20 (active) and Q4 (sham) cohorts (P<0.01). In contrast, the rate of pain decrease for patients treated with the 5/20 (active) regimen was not significantly different from those receiving no PEMF treatment in the Q4 (sham) cohort (P=0.271). Specifically, pain at 24 h postoperative was, respectively, 43% and 35% of pain at 1 h postoperative for patients in the Q4 (active) and Q2 (active) cohorts (P<0.01). In contrast, pain at 24 h for patients in the 5/20 (active) cohort was 87% of pain at 1 h, compared with 74% for patients in the Q4 (sham) cohort (P=0.451). These results can be seen in FIG. 4. A similar pattern of results was found in narcotic usage. Postoperative narcotic usage by 24 h postoperative for patients in the 5/20 (active) cohort was not significantly different from that in the Q4 (sham) cohort (P=0.478), and both were approximately 2-fold higher compared with narcotic usage for patients in the Q4 (active) and Q2 (active) cohorts (P<0.02). Narcotic usage for patients in the Q2 (active) and Q4 (active) cohorts was not significantly different (P=0.246). These results can be seen in FIG. 40.

The identical PEMF signal configuration was used for all active cohorts; however, the dosing regimen was different. Entry criteria were identical for all patients. Surgery was performed by the same surgeon on all patients. The results clearly suggest that the effectiveness of PEMF therapy on the rate of postoperative pain decrease and post-operative narcotic requirements in BR patients depends on PEMF dosing regimen. A 5/20 (active) regimen was no different than the Q4 (sham) regimen for pain reduction, whereas a Q2 (active) regimen was as effective as the Q4 (active) regimen.

The findings revealed that the regimen of PEMF can significantly impact its effect on postoperative pain. It was expected that the most frequent dosing at 5 min every 20 min would have the greatest effect on pain reduction, but this was not the case. Mean VAS pain scores for patients in the 5/20 (active) cohort were not significantly different from those for patients in the Q4 (sham) cohort, which were more than two-fold higher at 24 h postoperatively than VAS scores for patients in the Q4 (active) and Q2 (active) cohorts. Similar comparative results were obtained for postoperative narcotic usage for patients in each of the active cohorts.

PEMF signal parameters, including repetition rate, were identical for all patients in active cohorts. The dosing change was treatment regimen. The rate of increase in CaM-dependent NO, and therefore cGMP, from PEMF in tissue for the 5/20 regimen is nearly 2.5-fold higher than that for the Q2 (active) and 4-fold higher than that for the Q4 (active) regimens. The NO/cGMP signaling pathway is a principal anti-inflammatory pathway. CaM-dependent PDE activity regulates this pathway by inhibiting cGMP. The PEMF signal used in this study is known to enhance NO/cGMP signaling, and to enhance CaM-dependent PDE activity [5-7]. It was proposed that the 5/20 regimen caused PDE activity to predominate, thereby inhibiting all the enhanced cGMP produced by PEMF. The result is no effect of PEMF on postoperative pain.

Two recent publications illustrate that PEMF effects depend on signal configuration. The first showed that the PEMF effect on breast cancer cell apoptosis was significant when the same waveform, applied for the same exposure time, repeated at 20 Hz but not at 50 Hz [35]. The second study showed that PEMF significantly reduced the expression of inflammatory markers, tumor necrosis factor, and nuclear factor-kappa beta in challenged macrophages when the same waveform, applied for the same exposure time, was repeated at 5 Hz, but not at 15 or 30 Hz [36]. In both studies, CaM-dependent NO/cGMP signaling modulates the expressions of these inflammatory markers [37,38], suggesting the effect of increased repetition rate is consistent with increased production of NO at a rate high enough for PDE inhibition of cGMP isoforms to predominate, thus blocking the PEMF effect. It is interesting to note that similar dosing effects have been observed in studies using low-level laser therapy, wherein the mechanism of action also involves NO/cGMP signaling [39]. Comparable with our study, low-level laser therapy improvement of neurologic performance in a mouse traumatic brain injury model depended on treatment regimen [40].

This study provides evidence that nonthermal radio frequency PEMF therapy can accelerate pain reduction and decrease pain medication requirements in the immediate postoperative period. The effect of PEMF regimen has been elucidated and effective regimens defined. Every 2 or 4 h dosing significantly decreases postoperative pain, whereas every 20-min dosing has no effect compared with placebo. The results of this study confirm dosing by which a given PEMF signal, configured to enhance the body's primary anti-inflammatory signaling pathway, CaM-dependent NO/cGMP, can accelerate postoperative pain relief.

### EXAMPLE 11

At the cellular level the effect of PEMF signal configuration on PDE inhibition of cGMP was examined in cell cultures. Cells were plated in DMEM containing low concentration (challenge) of fetal calf-serum in 24-well plates. Two cell types were tested; human fibroblasts (HFC) and human chondrocytes (HCC). Cells were grown for 24-hours to allow for attachment and repair after initial plating. Cells were exposed to PEMF signals for 15 minutes Immediately after treatment conditioned media (CM) was collected and assayed for NO levels using the Griess reaction combined with vanadium. The Griess assay tests for nitrite (NO2-). Vanadium was used to reduce nitrate (NO3-) to NO2- since NO immediately reacts with water to form NO3- and NO2- and the Griess assay only measures NO2-.

Two PEMF signals with 27.12 MHz sinusoidal carrier were tested. One PEMF signal had a burst width of 2 msec repeating at 2 Hz (Signal I) while the second signal (Signal II) had a burst width of 3 msec repeating at 5 Hz. Both signals were tested at amplitudes ranging from 0.5 to 8 μT. Exposure time was 15 min for each amplitude condition. Since each waveform was configured to target Ca/CaM binding, Signal II would be expected to produce approximately 4-fold more NO than that produced by Signal I, at all amplitudes studied.

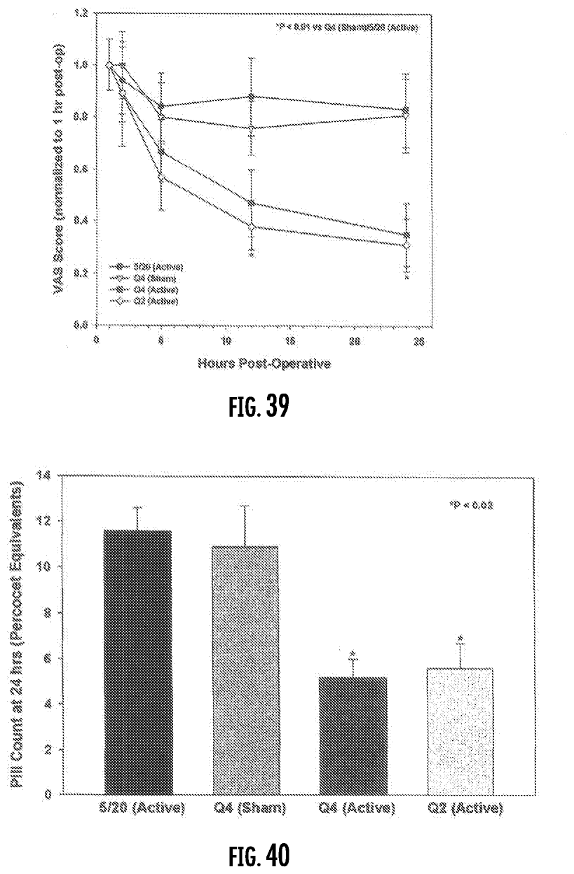

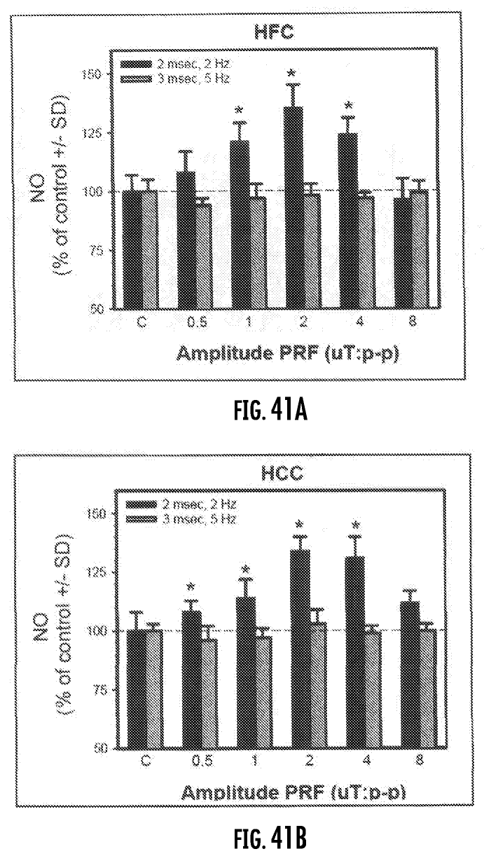

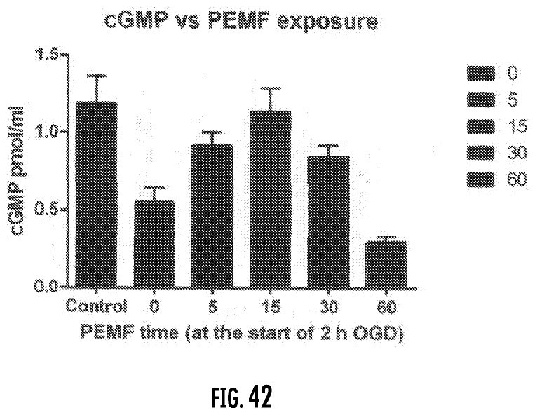

The results are shown in FIGS. 41A and 41B, wherein it may be seen that Signal I produced significant increases in NO over a range of amplitudes. In contrast, Signal II did not produce increased NO at any amplitude tested. As for the clinical example CaM-dependent PDE activity regulates NO/cGMP signaling by inhibiting cGMP. It was proposed that Signal II with increased burst duration and repetition rate compared to Signal I increased NO too rapidly causing the PEMF effect on PDE activity to predominate, thereby inhibiting all the enhanced NO produced by PEMF. The result is no effect of PEMF on NO release in challenged fibroblasts and chondrocytes for Signal II at any amplitude tested.

### EXAMPLE 12

At the cellular level the effect of PEMF signal configuration on PDE inhibition of cGMP was examined in cell cultures. Cells were plated in DMEM containing low concentration (challenge) of fetal calf-serum in 24-well plates. Two cell types were tested; human fibroblasts (HFC) and human chondrocytes (HCC). Cells were grown for 24-hours to allow for attachment and repair after initial plating. Cells were exposed to PEMF signals for 15 minutes Immediately after treatment conditioned media (CM) was collected and assayed for NO levels using the Griess reaction combined with vanadium. The Griess assay tests for nitrite (NO2-). Vanadium was used to reduce nitrate (NO3-) to NO2- since NO immediately reacts with water to form NO3- and NO2- and the Griess assay only measures NO2-.

Two PEMF signals with 27.12 MHz sinusoidal carrier were tested. One PEMF signal had a burst width of 2 msec repeating at 2 Hz (Signal I) while the second signal (Signal II) had a burst width of 3 msec repeating at 5 Hz. Both signals were tested at amplitudes ranging from 0.5 to 8 μT. Exposure time was 15 min for each amplitude condition. Since each waveform was configured to target Ca/CaM binding, Signal II would be expected to produce approximately 4-fold more NO than that produced by Signal I, at all amplitudes studied.

The results are shown in FIGS. 41A and 41B, wherein it may be seen that Signal I produced significant increases in NO over a range of amplitudes. In contrast, Signal II did not produce increased NO at any amplitude tested. As for the clinical example CaM-dependent PDE activity regulates NO/cGMP signaling by inhibiting cGMP. It was proposed that Signal II with increased burst duration and repetition rate compared to Signal I increased NO too rapidly causing the PEMF effect on PDE activity to predominate, thereby inhibiting all the enhanced NO produced by PEMF. The result is no effect of PEMF on NO release in challenged fibroblasts and chondrocytes for Signal II at any amplitude tested.

### EXAMPLE 13

In another cellular study the effect of exposure time of a PEMF signal consisting of a 2 msec burst of a 27.12 MHz carrier repeating at 2 Hz and delivering 4 μT amplitude was tested. Primary neuronal cells were subjected to oxygen glucose deprivation (OGD) which subjects the cells to ischemic conditions such as those which exist in cardiac and brain ischemia. OGD is expected to reduce NO and therefore cGMP. The parameters of the PEMF signal were chosen to modulate CaM/NO/cGMP signaling so that exposure to PEMF during OGD would be expected to produce increased cGMP. However, as exposure time increases, the PEMF effect on PDE activity also increases. Referring to the results given in the previous clinical and cellular examples, an exposure time of 60 minutes would be expected to produce about 4-fold more NO than the standard effective 15 minute exposure. The results are shown in FIG. 7 wherein it may be seen that 15 minute PEMF exposure maximally restored cGMP production. In contrast, an exposure time of 60 minutes was not effective.

Therefore, in some embodiments any of the PEMF parameters can be selected to minimize the PDE inhibition of cGMP and/or maximize the production of cGMP. In some embodiments the length of the inter-treatment period and the length of the treatment interval are selected to minimize the PDE inhibition of cGMP. Any of the PEMF waveform parameters can be optimized in conjunction with the length of the treatment interval and inter-treatment period to achieve a desired change to the production of PDE and/or to decrease the inhibition of cGMP by PDE.

In the example shown in FIG. 36B, once treatment begins 36103, the device, in some variations, applies an envelope of high-frequency waveforms at low amplitude (e.g. less than 50 milliGauss, less than 100 milliGauss, less than 200 milliGauss, etc.) 36105. This envelope of high-frequency pulses is then repeated at a particular frequency (repetition rate) after an appropriate delay. The repetition rate may be varied to minimize PDE inhibition of PDE. The amplitude may be varied to minimize PDE inhibition of PDE. The burst duration may be varied to minimize PDE inhibition of PDE.

The initial signal configuration (burst duration, burst repetition and amplitude) can be repeated for a first treatment time and then followed by a delay during which the treatment is “off” 107. This waiting interval (inter-treatment interval) may last for minutes or hours and then the treatment interval may be repeated again until the treatment regime is complete 36109.

In some embodiments the length of the inter-treatment period can be selected to minimize the PDE inhibition of cGMP. In some embodiments the inter-treatment period is greater than about 15 minutes. In some embodiments the inter-treatment period is greater than about 30 minutes. In some embodiments the inter-treatment period is greater than about 60 minutes. In some embodiments the inter-treatment period is greater than about 90 minutes. In some embodiments the inter-treatment period is greater than about 120 minutes. In some embodiments the inter-treatment period is greater than about 180 minutes. In some embodiments the inter-treatment period is greater than about 240 minutes

In some embodiments the inter-treatment period can be expressed as a multiple of the PEMF treatment interval. In some embodiments the inter-treatment period is at least three times longer than the treatment interval. In some embodiments the inter-treatment period is at least four times longer than the treatment interval. In some embodiments the inter-treatment period is at least five times longer than the treatment interval. In some embodiments the inter-treatment period is at least six times longer than the treatment interval. In some embodiments the inter-treatment period is at least seven times longer than the treatment interval. In some embodiments the inter-treatment period is at least eight times longer than the treatment interval. In some embodiments the inter-treatment period is at least ten times longer than the treatment interval. In some embodiments the inter-treatment period is at least fifteen times longer than the treatment interval. In some embodiments the inter-treatment period is at least twenty times longer than the treatment interval.

Any of the PEMF treatment intervals disclosed herein can be used with any of the inter-treatment intervals disclosed herein. In some embodiments the treatment interval is about 5 minutes or longer. In some embodiments the treatment interval is about 10 minutes or longer. In some embodiments the treatment interval is about 15 minutes or longer. In some embodiments the treatment interval is about 20 minutes or longer.

In some embodiments the PEMF treatment period is five minutes with a 15 minute inter-treatment period. In some embodiments the PEMF treatment period is 15 minutes with a 105 minute inter-treatment period (e.g. 15 minutes of PEMF treatment per two hours). In some embodiments the PEMF treatment period is 20 minutes with a 160 minute inter-treatment period (e.g. 20 minutes of PEMF treatment per three hours).

In some variations, the treatment device is pre-programmed (or configured to receive pre-programming) to execute the entire treatment regime (including multiple on-periods and/or intra-treatment intervals) punctuated by predetermined off-periods (inter-treatment intervals) when no treatment is applied. In further variations, the device is pre-programmed to emit a PEMF signal at 27.12 MHz at 2 msec bursts repeating at 2 bursts/sec. In other embodiments, the device is pre-programed to emit a PEMF signal at 27.12 MHz (at about amplitude 250-400 mV/cm) pulsed in 4 msec bursts at 2 Hz.

In further variations, the method may include a pulsed electromagnetic field comprising a 2 msec burst of 27.12 MHz sinusoidal waves repeating at 2 Hz. In other variations, the method may include a pulsed electromagnetic field comprising a 3 msec burst of 27.12 MHz sinusoidal waves repeating at 2 Hz. In further embodiments, the pulsed electromagnetic field may comprise a 4 msec burst of 27.12 MHz sinusoidal waves repeating at 2 Hz.

The patient can be monitored during the PEMF treatment regime to determine the physiological response to the PEMF treatment regime. The treatment cycle (e.g. treatment period and inter-treatment period) can be repeated until a desired physiological response is achieved. Depending on the patient's response to the treatment, the subsequent treatment cycle parameters can be adjusted by a health professional to achieve a desired physiological response in the patient.

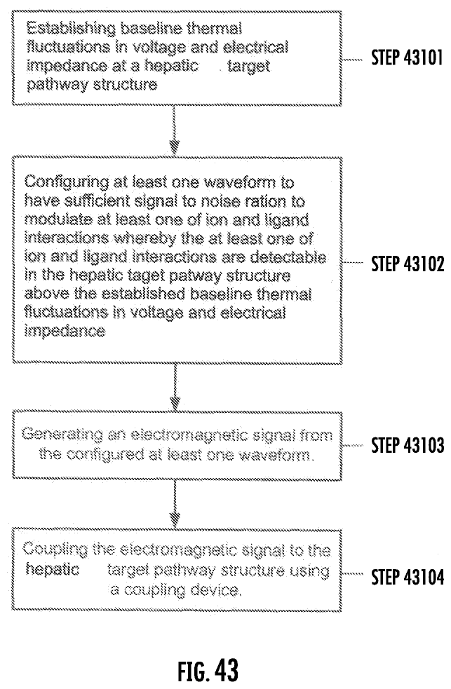

Referring to FIG. 43 wherein FIG. 43 is a flow diagram of a method for generating electromagnetic signals to be coupled to a hepatic target pathway structure according to an embodiment of the present invention, a target pathway structure such as ions and ligands, is identified. Establishing a baseline background activity such as baseline thermal fluctuations in voltage and electrical impedance, at the target pathway structure by determining a state of at least one of a cell and a tissue at the target pathway structure, wherein the state is at least one of resting, growing, replacing, and responding to injury. (STEP 43101) The state of the at least one of a cell and a tissue is determined by its response to injury or insult. Configuring at least one waveform to have sufficient signal to noise ratio to modulate at least one of ion and ligand interactions whereby the at least one of ion and ligand interactions are detectable in the target pathway structure above the established baseline thermal fluctuations in voltage and electrical impedance. (STEP 43102) Repetitively generating an electromagnetic signal from the configured at least one waveform. (STEP 43103) The electromagnetic signal can be generated by using at least one waveform configured by applying a mathematical model such as an equation, formula, or function having at least one waveform parameter that satisfies an SNR or Power SNR mathematical model such that ion and ligand interactions are modulated and the at least one configured waveform is detectable at the target pathway structure above its established background activity. Coupling the electromagnetic signal to the target pathway structure using a coupling device. (STEP 43104) The generated electromagnetic signals can be coupled for therapeutic and prophylactic purposes. The coupling enhances a stimulus that cells and tissues react to in a physiological meaningful manner for example, treatment of non-alcoholic steatohepatitis.

In an aspect of the present invention, a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model. A repetitive electromagnetic signal can be generated for example inductively or capacitively, from the configured at least one waveform. The electromagnetic signal is coupled to a target pathway structure such as ions and ligands by output of a coupling device such as an electrode or an inductor, placed in close proximity to the target pathway structure using a positioning device. The coupling enhances modulation of binding of ions and ligands to regulatory molecules, tissues, cells, and organs. According to an embodiment of the present invention EMF signals configured using SNR analysis to match the bandpass of a second messenger whereby the EMF signals can act as a first messenger to modulate biochemical cascades such as production of cytokines, Nitric Oxide, Nitric Oxide Synthase and growth factors that are related to tissue growth and repair. A detectable E field amplitude is produced within a frequency response of Ca2+ binding.

An embodiment according to the present invention applies a mathematical model to induce a time-varying magnetic field and a time-varying electric field in a target pathway structure such as ions and ligands, comprising about 0.001 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency.

A waveform configured using an embodiment according to the present invention may be applied to a target pathway structure such as ions and ligands, preferably for a total exposure time of under 1 minute to 240 minutes daily. However other exposure times can be used. Waveforms configured by a miniature control circuit, which may be similar or identical to the control circuit 300 shown in FIG. 3, are directed to a generating device such as electrical coils.

Preferably, the generating device is a conformable coil for example pliable, comprising one or more turns of electrically conducting wire in a generally circular or oval shape however other shapes can be used. The generating device 32202 delivers a pulsing magnetic field configured according to a mathematical model that can be used to provide treatment to a target pathway structure such as hepatic tissue. The miniature control circuit applies a pulsing magnetic field for a prescribed time and can automatically repeat applying the pulsing magnetic field for as many applications as are needed in a given time period, for example 12 times a day. The miniature control circuit can be configured to be programmable applying pulsing magnetic fields for any time repetition sequence. An embodiment according to the present invention can be positioned to treat liver disease tissue by being incorporated with a positioning device that can be wrapped around a patient's torso. Coupling a pulsing magnetic field to a target pathway structure such as ions and ligands, therapeutically and prophylactically reduces inflammation thereby reducing pain and promotes healing in treatment areas. When electrical coils are used as the generating device, the electrical coils can be powered with a time varying magnetic field that induces a time varying electric field in a target pathway structure according to Faraday's law. An electromagnetic signal generated by the generating device can also be applied using electrochemical coupling, wherein electrodes are in direct contact with skin or another outer electrically conductive boundary of a target pathway structure.

Yet in another embodiment according to the present invention, the electromagnetic signal generated by the generating device can also be applied using electrostatic coupling wherein an air gap exists between a generating device such as an electrode and a target pathway structure such as ions and ligands. An advantage of the present invention is that its ultra-lightweight coils and miniaturized circuitry allow for use with common physical therapy treatment modalities, and at any location for which tissue growth, pain relief, and tissue and organ healing is desired. An advantageous result of application of the present invention is that tissue growth, repair, and maintenance can be accomplished and enhanced anywhere and at any time. Yet another advantageous result of application of the present invention is that growth, repair, and maintenance of molecules, cells, tissues, and organs can be accomplished and enhanced anywhere and at any time.

### EXAMPLE 14

FIG. 44 shows a test apparatus 4410 used to study the efficacy of electromagnetic pulse therapy (PEMF) in a mouse model of nonalcoholic steathohepatitis (NASH). The apparatus 4410 comprises a plastic base pan 4412 including a floor 4414 and a plurality of side walls 4416. An applicator coil 4418 extends along the entire perimeter of the base pan 4412, parallel to and 3.5″ above the floor 4414. A BNC cable 4417 couples the applicator coil 4418 to an electromagnetic field source that generates a pulsed magnetic field. A radiofrequency probe 4420 mounted in the center of the base pan 4412 is electrically coupled to an oscilloscope that displays the signal voltages.

The study was conducted using 31 male C57LB/6 mice from Charles River Laboratories, all approximately 8 weeks old. The mouse model of NASH was created by feeding the mice a diet deficient in methionine and choline (MCD diet), the ingredients of which are listed under the product name A2082002BR in the table below:

TABLE 1
Methionine and Choline Deficient L-
Amino Acid Diet and Control Diet

Rt

| | Product # | | | |
|---|---|---|---|---|
| | A02082002BR | | A02082003BY | |
| | gm | kcal | gm | kcal |
| Protein | 17 | 16 | 17 | 16 |
| Carbohydrate | 66 | 63 | 65 | 62 |
| Fat | 10 | 21 | 10 | 21 |
| | | 100 | | 100 |
| | 4.2 | | 4.2 | |
| Ingredient (gm) | | | | |
| L-Alanine | 3.5 | 14 | 3.5 | 14 |
| L-Arginine | 12.1 | 48.4 | 12.1 | 48.4 |
| L-Asparagine-H2O | 6 | 24 | 6 | 24 |
| L-Aspartate | 3.5 | 14 | 3.5 | 14 |
| L-Cystine | 3.5 | 14 | 3.5 | 14 |
| L-Glutamine | 40 | 160 | 40 | 160 |
| Glycine | 23.3 | 93.2 | 23.3 | 93.2 |
| L-Histidine-HCl-H2O | 4.5 | 18 | 4.5 | 18 |
| L-Isoleucine | 8.2 | 32.8 | 8.2 | 32.8 |
| L-Leucine | 11.1 | 44.4 | 11.1 | 44.4 |
| L-Lysine-HCl | 18 | 72 | 18 | 72 |
| L-Phenylalanine | 7.5 | 30 | 7.5 | 30 |
| L-Proline | 3.5 | 14 | 3.5 | 14 |
| L-Serine | 3.5 | 14 | 3.5 | 14 |
| L-Threonine | 8.2 | 32.8 | 8.2 | 32.8 |
| L-Tryptophan | 1.8 | 7.2 | 1.8 | 7.2 |
| L-Tyrosine | 5 | 20 | 5 | 20 |
| L-Valine | 8.2 | 32.8 | 8.2 | 32.8 |
| Total L-Amino Acids | 171.4 | 685.6 | 171.4 | 685.6 |
| Sucrose | 455.3 | 1821.2 | 452.3 | 1809.2 |
| Corn Starch | 150 | 600 | 150 | 600 |
| Maltodextrin 10 | 50 | 200 | 50 | 200 |
| Cellulose | 30 | 0 | 30 | 0 |
| Corn Oil | 100 | 900 | 100 | 900 |
| Mineral Mix S10001 | 35 | 0 | 35 | 0 |
| Sodium Bicarbonate | 7.5 | 0 | 7.5 | 0 |
| Vitamin Mix V10001 | 10 | 40 | 10 | 40 |
| L-Methionine | 0 | 0 | 3 | 12 |
| Choline Bitrartrate | 0 | 0 | 2 | 0 |
| FD&C Red Dye #40 | 0.05 | 0 | 0 | 0 |
| FD&C Yellow Dye #5 | 0 | 0 | 0.05 | 0 |
| Total | 1009.25 | 4247 | 1011.25 | 4247 |

The mice were divided into a naïve group (Group 1) consisting of 3 animals, 2 control groups (Groups 2 and 4) consisting of 7 animals each, and 2 active treatment groups (Groups 3 and 5) consisting of 7 animals each. Group 1 was fed a normal diet (identified as product A2082003BY in Table 1) and did not receive any treatment. Groups 2-5 were fed the MCD diet. Groups 2 and 4 received a sham treatment for 7 and 14 days, respectively, while Groups 3 and 5 received PEMF therapy for 7 and 14 days, respectively. The treatments were given to two groups simultaneously, one group would be contained in a first all-plastic cage centered within the base pan 4412 of a first treatment apparatus 4410 by foam blocks 44 22, and the second group in a second all-plastic cage centered within the base pan of a second treatment apparatus. The treatments were then be repeated using the other two groups.

The PEMF therapy comprised placing the mice in the treatment apparatus and generating a PEMF signal in the applicator coil. The PEMF signal was a sinusoidal wave at 27.12 MHz delivered in 2 msec bursts at a frequency of 2 bursts per second, with a field strength of 5 μT. Each treatment was given for 15 minutes at 3 hour intervals, three times per day. The sham treatment comprised placing the mice in the treatment apparatus and sending a null signal through the applicator coil.

Animals fed on the MCD diet but subjected to sham treatment showed (compared to “naïve” animal fed with standard chow) symptoms consistent with liver dysfunction, including:
Increased liver inflammation and liver cell ballooning
Body weight loss (up to 25% by the second week)
Decreased blood glucose levels (up to 2-fold)
Increased blood levels of liver enzymes alanine transaminase (ALT) and aspartate transaminase (AST) (22- and 8-fold, respectively)
Decreased blood lipids, including cholesterol, triglycerides, and high-density lipoprotein (HDL) (2- to 4-fold).

These results demonstrate the validity of the model in causing liver damage. Notably, the effects on liver cell inflammation and hepatocyte ballooning are consistent with the early stages of NASH.

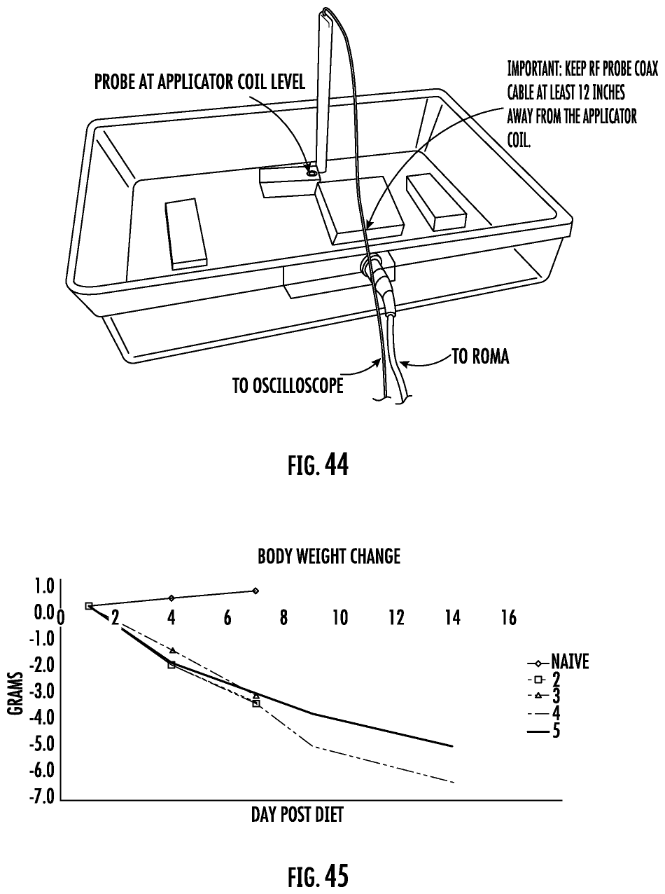

Changes in weight according to different treatment regimes are shown graphically in FIG. 45. The animals in Groups 1-5 were weighed on the 1^{st}, 4^{th} and 7^{th} days of treatment, and the animals in Groups in 4 and 5 were also weighed on the 9^{th} and 14^{th} days. As seen in the graph of FIG. 45 and in Table 2 below, the animals given the control diet (A2082003BY) gained weight over a 7-day period, while all the animals given the MCD diet lost weight. The animals receiving PEMF treatment lost less weight than those receiving the sham treatment, with the 14-day PEMG treatment resulting in less weight loss than the 7-day PEMG treatment.

TABLE 2

| | Day-1 | Day-4 | Day-7 | Day-9 | Day-14 |
|---|---|---|---|---|---|
| Mouse# | BW | BW | BW | BW | BW |
| 1-1 | 25.8 | 26 | 26 | | |
| 1-2 | 27.3 | 27.5 | 28.2 | | |
| 1-3 | 27.4 | 27.9 | 28.1 | | |
| 2-1 | 22.7 | 20.8 | 19 | | |
| 2-2 | 25.7 | 22.8 | 22 | | |
| 2-3 | 25.5 | 23.2 | 21.4 | | |
| 2-4 | 26.5 | 24.7 | 23.1 | | |
| 2-5 | 27.1 | 24.8 | 23.3 | | |
| 2-6 | 26.1 | 25.1 | 23.8 | | |
| 2-7 | 27.8 | 25.3 | 23.9 | | |
| 3-1 | 23.1 | 21 | 19.7 | | |
| 3-2 | 27.2 | 25.4 | 23.5 | | |
| 3-3 | 26.2 | 24 | 21.8 | | |
| 3-4 | 22.3 | 21.1 | 19.8 | | |
| 3-5 | 26.3 | 25.1 | 22.5 | | |
| 3-6 | 25.4 | 24.1 | 22.7 | | |
| 3-7 | 22.4 | 21 | 19.6 | | |
| 4-1 | 25.6 | 23.5 | 21.8 | 21 | 19.4 |
| 4-2 | 24 | 21.7 | 20 | 18.9 | 17.8 |
| 4-3 | 24.9 | 23.1 | 21.9 | 20.6 | 19.5 |
| 4-4 | 28.3 | 25.6 | 24.1 | 22.6 | 21.1 |
| 4-5 | 25.7 | 24.3 | 22.8 | 21.4 | 20.3 |
| 4-6 | 28.4 | 26.2 | 24 | 22.3 | 20.7 |
| 4-7 | 26 | 23.9 | 22.7 | 20.4 | 19.1 |
| 5-1 | 26.2 | 24.6 | 22.9 | 21.9 | 21.4 |
| 5-2 | 24.2 | 21.6 | 20.7 | 19.2 | 17.9 |
| 5-3 | 23.9 | 22.3 | 21 | 20.4 | 19 |
| 5-4 | 23.8 | 21.4 | 20 | 19.6 | 19.8 |
| 5-5 | 24.4 | 22.1 | 21.9 | 21.1 | 19 |
| 5-6 | 23.3 | 22.3 | 21 | 20.2 | 18.3 |
| 5-7 | 26.6 | 24.1 | 22.7 | 22.6 | 21.5 |

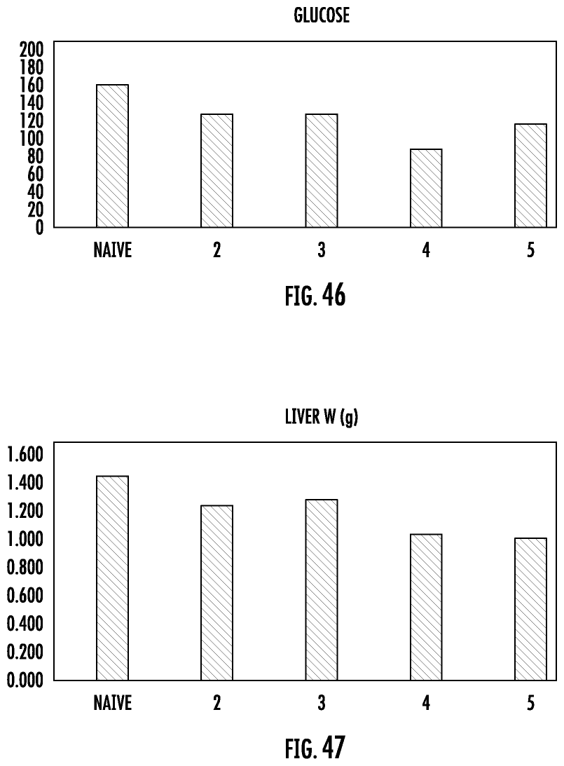

The animals in each group were sacrificed approximately 2 hours after their last treatment, and their levels of glucose, Alanine aminotransferase (ALT), and Aspartate aminotransferase (AST) were measured. Results of the glucose, ALT, and AST readings are shown in Table 3 below. The glucose levels are shown graphically in FIG. 46.

TABLE 3

| Mouse# | Glucose | ALT (GPT) | AST (GOT) |
|---|---|---|---|
| 1-1 | 151 | 23 | 66 |
| 1-2 | 200 | 25 | 51 |
| 1-3 | 166 | 33 | 57 |
| 2-1 | 118 | 216 | 220 |
| 2-2 | 158 | 589 | 584 |
| 2-3 | 138 | 339 | 328 |
| 2-4 | 156 | 915 | 582 |
| 2-5 | 186 | 415 | 364 |
| 2-6 | 93 | 293 | 316 |
| 2-7 | 110 | 680 | 387 |
| 3-1 | 153 | 291 | 198 |
| 3-2 | 153 | **** | **** |
| 3-3 | 209 | 261 | 203 |
| 3-4 | 153 | 266 | 255 |
| 3-5 | 161 | 169 | 183 |
| 3-6 | 21 | 236 | 297 |
| 3-7 | 120 | 378 | 836 |
| 4-1 | 129 | 549 | 330 |
| 4-2 | 105 | 482 | 505 |
| 4-3 | 124 | 672 | 446 |
| 4-4 | 95 | 620 | 758 |
| 4-5 | 76 | 530 | 408 |
| 4-6 | 73 | 637 | 427 |
| 4-7 | 77 | 723 | 419 |
| 5-1 | 141 | 603 | 346 |
| 5-2 | 150 | 1000 | 589 |
| 5-3 | 133 | 669 | 618 |
| 5-4 | 126 | **** | **** |
| 5-5 | 135 | 1000 | 856 |
| 5-6 | 120 | 655 | 868 |
| 5-7 | 114 | 505 | 342 |

Plasma lipid panel readings were also taken, and are shown in in Table 4 below.

TABLE 4

| | | | HDL | LDL |
|---|---|---|---|---|
| | Cholesterol | Triglycerides | Cholesterol | Cholesterol |
| Mouse# | (mg/dL) | (mg/dL) | (mg/dL) | (mg/dL) |
| 1-1 | 115 | 126 | 70 | 8 |
| 1-2 | 113 | 175 | 77 | 7 |
| 1-3 | 145 | 206 | 93 | 8 |
| 2-1 | 74 | 102 | 45 | 8 |
| 2-2 | 65 | 84 | 38 | 8 |
| 2-3 | 77 | 100 | 44 | 11 |
| 2-4 | 65 | 73 | 35 | 9 |
| 2-5 | 80 | 92 | 46 | 11 |
| 2-6 | 67 | 83 | 36 | 9 |
| 2-7 | 78 | 80 | 45 | 11 |
| 3-1 | 79 | 94 | 44 | 11 |
| 3-2 | 86 | 94 | 49 | 11 |
| 3-3 | 90 | 113 | 50 | 12 |
| 3-4 | 80 | 85 | 45 | 10 |
| 3-5 | 95 | 111 | 43 | 11 |
| 3-6 | 86 | 106 | 49 | 11 |
| 3-7 | 75 | 106 | 42 | 10 |
| 4-1 | 51 | 90 | 26 | 7 |
| 4-2 | 39 | 71 | 16 | 7 |
| 4-3 | 41 | 70 | 20 | 7 |
| 4-4 | 45 | 77 | 21 | 7 |
| 4-5 | 52 | 76 | 25 | 7 |
| 4-6 | 40 | 77 | 18 | 7 |
| 4-7 | 40 | 75 | 15 | 7 |
| 5-1 | 39 | 115 | 13 | 7 |
| 5-2 | 59 | 105 | 25 | 12 |
| 5-3 | 50 | 86 | 22 | 8 |
| 5-4 | 53 | 83 | 24 | 8 |
| 5-5 | 55 | 88 | 20 | 13 |
| 5-6 | 44 | 68 | 15 | 7 |
| 5-7 | 35 | 73 | 14 | 7 |

The liver of each animal was weighed, and the total weight was recorded as shown in Table 5 below, and illustrated in the bar graph of FIG. 47. The medial lobe of each liver was then excised, formalin fixed, and stained with Oil red O to evaluate lipid content. The intensity of hepatocyte ballooning was subjectively assessed and reported as shown in Table 6 below. As can be seen from the bar graph of FIG. 5, the livers of animals receiving PEMF showed less ballooning than their counterparts who received sham treatment over the same period of time.

TABLE 5

| Mouse# | Liver W (g) | Takedown day | |
|---|---|---|---|
| 1-1 | 1.289 | 7 | d |
| 1-2 | 1.465 | 7 | d |
| 1-3 | 1.573 | 7 | d |
| 2-1 | 1.029 | 7 | d |
| 2-2 | 1.233 | 7 | d |
| 2-3 | 1.237 | 7 | d |
| 2-4 | 1.31 | 7 | d |
| 2-5 | 1.321 | 7 | d |
| 2-6 | 1.3 | 7 | d |
| 2-7 | 1.278 | 7 | d |
| 3-1 | 1.184 | 7 | d |
| 3-2 | 1.384 | 7 | d |
| 3-3 | 1.292 | 7 | d |
| 3-4 | 1.289 | 7 | d |
| 3-5 | 1.243 | 7 | d |
| 3-6 | 1.479 | 7 | d |
| 3-7 | 1.098 | 7 | d |
| 4-1 | 1.024 | 14 | d |
| 4-2 | 0.917 | 14 | d |
| 4-3 | 0.971 | 14 | d |
| 4-4 | 1.095 | 14 | d |
| 4-5 | 1.1 | 14 | d |
| 4-6 | 1.096 | 14 | d |
| 4-7 | 1.113 | 14 | d |
| 5-1 | 1.128 | 14 | d |
| 5-2 | 0.905 | 14 | d |
| 5-3 | 1.006 | 14 | d |
| 5-4 | 1.336 | 14 | d |
| 5-5 | 1.051 | 14 | d |
| 5-6 | 0.872 | 14 | d |
| 5-7 | 0.918 | 14 | d |

TABLE 6

| | section | section | section | section | section | section | section | section | section | section |
|---|---|---|---|---|---|---|---|---|---|---|
| mouse# | 1 | 2 | 3 | 4 | 5 | 6 | 7 | 8 | 9 | 10 |
| 1-1 | 0 | 0 | 0 | 0 | 0 | 0 | 0 | 0 | 0 | 0 |
| 1-2 | 0 | 0 | 0 | 0 | 0 | 0 | 0 | 0 | 0 | 0 |
| 1-3 | 0 | 0 | 0 | 0 | 0 | 0 | 0 | 0 | 0 | 0 |
| 2-1 | 1 | 1 | 1 | 1 | 1 | 1 | 1 | 1 | 1 | 1 |
| 2-2 | 1 | 1 | 1 | 1 | 1 | 1 | 1 | 1 | 1 | 1 |
| 2-3 | 1.5 | 1.5 | 1.5 | 1.5 | 1.5 | 1.5 | 1.5 | 1.5 | 1.5 | 1.5 |
| 2-4 | 1.5 | 2 | 2 | 1.5 | 1.5 | 1.5 | 1.5 | 1.5 | 2 | 1.5 |
| 2-5 | 1 | 1.5 | 1 | 1.5 | 1.5 | 1 | 1 | 1 | 1 | 1 |
| 2-6 | 2 | 1.5 | 1.5 | 1.5 | 1.5 | 1.5 | 1.5 | 1.5 | 1.5 | 1.5 |
| 2-7 | 1.5 | 1.5 | 1.5 | 1.5 | 1.5 | 1.5 | 1.5 | 1.5 | 1.5 | 1.5 |
| 3-1 | 1 | 1 | 1.5 | 1.5 | 1 | 1 | 1 | 1.5 | 1 | 1.5 |
| 3-2 | 1.5 | 1.5 | 1.5 | 1.5 | 1.5 | 1.5 | 1.5 | 1.5 | 1.5 | 1.5 |
| 3-3 | 1 | 1 | 1 | 1 | 1 | 1 | 1 | 1 | 1 | 1 |
| 3-4 | 1 | 1 | 1 | 1 | 1 | 1 | 1 | 1 | 1 | 1 |
| 3-5 | 1 | 1 | 1 | 1 | 1 | 1 | 1 | 1 | 1 | 1 |
| 3-6 | 1 | 1 | 1 | 1 | 1 | 1 | 1 | 1 | 1 | 1 |
| 3-7 | 1 | 1 | 1 | 1 | 1 | 1 | 1 | 1 | 1 | 1 |
| 4-1 | 3 | 3 | 3 | 3 | 3 | 3 | 2 | 3 | 2 | 2 |
| 4-2 | 2 | 2 | 2.5 | 2 | 2 | 2.5 | 2 | 2 | 2 | 2 |
| 4-3 | 3 | 2 | 3 | 3 | 2 | 2 | 2.5 | 2.5 | 2 | 2 |
| 4-4 | 2.5 | 2.5 | 3 | 3 | 2 | 2.5 | 2 | 2.5 | 3 | 2 |
| 4-5 | 1 | 2 | 2 | 1 | 1 | 2 | 2 | 1 | 1 | 2 |
| 4-6 | 1 | 1 | 2 | 1 | 2 | 1 | 1 | 2 | 1 | 1 |
| 4-7 | 3 | 2 | 3 | 3 | 2 | 3 | 3 | 2 | 2 | 2 |
| 5-1 | 1 | 1 | 1 | 1 | 1 | 1 | 1 | 1 | 1 | 1 |
| 5-2 | 1 | 1 | 1 | 1 | 1 | 1 | 1 | 1 | 1 | 1 |
| 5-3 | 2 | 2 | 3 | 3 | 3 | 2 | 2 | 3 | 2 | 2 |
| 5-4 | 2 | 2 | 3 | 3 | 2 | 3 | 2 | 2 | 3 | 2 |
| 5-5 | 1 | 1 | 1 | 2 | 3 | 2 | 2 | 2 | 2 | 2 |
| 5-6 | 1 | 1 | 1 | 1 | 1 | 1 | 1 | 1 | 1 | 1 |
| 5-7 | 1 | 1 | 1 | 1 | 1 | 1 | 1 | 1 | 1 | 1 |

In summary, animals fed on the MCD diet and subjected to treatment with the Endonovo Therapeutics device showed (compared to sham-treated MCD-fed animals) limited relief of disease symptoms, including:
Nominal decreases in liver inflammation and liver cell ballooning
Attenuation of body weight loss (at 2 weeks)
Restoration of blood glucose levels (at 2 weeks) and blood lipid levels (at 1 week)
Nominal decreases in blood levels of liver enzymes ALT and AST (at 1 week)

Thus, 1 to 2 weeks of treatment with the non-invasive PEMF treatment appeared to counteract several of the disease signs associated with NASH-associated liver damage.

### LIST OF REFERENCES CITED HEREIN

[1] Kehlet H, Wilmore D. Evidence-based surgical care and the evolution of fast-track surgery. Ann Surg 2008; 248:189. [0082] [2] Binshtok A M, Wang H, Zimmermann K, et al. Nociceptors are interleukin-lbeta sensors. J Neurosci 2008; 28:14062. [0083] [3] Pilla A A. Mechanisms and therapeutic applications of time varying and static magnetic fields. In: Barnes F, GreenebaumB, editors. Biological and Medical Aspects of Electromagnetic Fields. Boca Raton Fla.: CRC Press; 2007. p. 351. [0084] [4] Pilla A A, Muehsam D J, Markov M S, Sisken B F. EMF signals and ion/ligand Binding kinetics: prediction of bioeffective waveform parameters. Bioelectrochem bioenerg 1999; 48:27. [0085] [5] Pilla A, Fitzsimmons R, Muehsam D, Wu J, Rohde C, Casper D. Electromagnetic fields as first messenger in biological signaling: application to calmodulin-dependent signaling in tissue repair. Biochim Biophys Acta 2011; 1810:1236. [0086] Pilla A A. Electromagnetic fields instantaneously modulate nitric oxide signaling in challenged biological systems. Biochem Biophys Res Commun 2012; 426:330. [0087] [7] Pilla A A. Nonthermal electromagnetic fields: from first messenger to therapeutic applications. Electromagn Biol Med 2013; 32:123. [0088] [8] Ren K, Torres R. Role of interleukin-lbeta during pain and inflammation. Brain Res Rev 2009; 60:57. [0089] [9] Werner S, Grose R. Regulation of wound healing by growth factors and cytokines. Physiol Rev 2003; 83:835. [0090] [10] Yen-Patton G P, Patton W F, Beer D M, et al. Endothelial cell response to pulsed electromagnetic fields: stimulation of growth rate and angiogenesis in vitro. J Cell Physiol 1988; 134:37. [0091] [11] Tepper O M, Callaghan M J, Chang E I, et al. Electromagnetic fields increase in vitro and in vivo angiogenesis through endothelial release of FGF-2. FASEB J 2004; 18:1231. [0092] [12] Callaghan M J, Chang E I, Seiser N, et al. Pulsed electromagnetic fields accelerate normal and diabetic wound healing by increasing endogenous FGF-2 release. Plast Reconstr Surg 2008; 121:130. [0093] [13] Roland D, Ferder M S, Kothuru R, Faierman T, Strauch B. Effects of pulsed magnetic energy on a microsurgically transferred vessel. Plast Reconstr Surg 2000; 105:1371. [0094] [14] Weber R V, Navarro A, Wu J K, Yu H L, Strauch B. Pulsed magnetic fields applied to a transferred arterial loop support the rat groin composite flap. Plast Reconstr Surg 2004; 114:1185. [0095] [15] Delle Monache S, Alessandro R, Iorio R, Gualtieri G, Colonna R. Extremely low frequency electromagnetic fields (ELF-EMFs) induce in vitro angiogenesis process in human endothelial cells. Bioelectromagnetics 2008; 29:640. [0096] [16] Rohde C, Chiang A, Adipoju O, Casper D, Pilla A A. Effects of pulsed electromagnetic fields on interleukin-1 beta and postoperative pain: a double-blind, placebo-controlled, pilot study in breast reduction patients. Plast Reconstr Surg 2010; 125:1620. [0097] [17] Hede'n P, Pilla A A. Effects of pulsed electromagnetic fields on postoperative pain: a double-blind randomized pilot study in breast augmentation patients. Aesthet Plast Surg 2008; 32:660. [0098] [18] Rawe I M, Lowenstein A, Barcelo C R, Genecov D G. Control of postoperative pain with a wearable continuously operating pulsed radiofrequency energy device: a preliminary study. Aesthet Plast Surg 2012; 36:458. [0099] [19] Rohde C, Hardy K, Asherman J, Taylor E, Pilla A A. PEMF therapy rapidly reduces post-operative pain in TRAM flap patients. Plast Reconstr Surg 2012; 130:91. [0100] [20] Strauch B, Herman C, Dabb R, Ignarro L J, Pilla A A. Evidencebased use of pulsed electromagnetic field therapy in clinical plastic surgery. Aesthet Surg J 2009; 29:135. [0101] [21] Guo L, Kubat N J, Nelson T R, Isenberg R A. Meta-analysis of clinical efficacy of pulsed radio frequency energy treatment. Ann Surg 2012; 255:457. [0102] [22] Batchelor A M, Bartus K, Reynell C, et al. Exquisite sensitivity to subsecond, picomolar nitric oxide transients conferred on cells by guanylyl cyclase-coupled receptors. Proc Natl Acad Sci USA 2010; 107:22060. [0103] [23] Mo E, Amin H, Bianco I H, Garthwaite J. Kinetics of a cellular nitric oxide/cGMP/phosphodiesterase-5 pathway. J Biol Chem 2004; 279:26149. [0104] [24] Miller C L, Oikawa M, Cai Y, et al. Role of Ca21p/calmodulin stimulated cyclic nucleotide phosphodiesterase 1 in mediating cardiomyocyte hypertrophy. Circ Res 2009; 105: 956. [0105] [25] Li Wan Po A, Petersen B. How high should total pain-relief score be to obviate the need for analgesic remedication in acute pain? Estimation using signal detection theory and individual-patient meta-analysis. J Clin Pharm Ther 2006; 31:161. [0106] [26] Wise R J. A preliminary report on a method of planning the mammaplasty. Plast Reconstr Surg 1956; 17:367. [0107] [27] Karp N S. Medial pedicle/vertical breast reduction made easy: the importance of complete inferior glandular resection. Ann Plast Surg 2004; 52:458. [0108] [28] Davison S P, Mesbahi A N, Ducic I, Sarcia M, Dayan J, Spear S L. The versatility of the superomedial pedicle with various skin reduction patterns. Plast Reconstr Surg 2007; 120:1466. [0109] [29] Rasouli J, Lekhraj R, White N M, et al. Attenuation of interleukin-lbeta by pulsed electromagnetic fields after traumatic brain injury. Neurosci Lett 2012; 519:4. [0110] [30] McLeod B R, Pilla A A, Sampsel M W. Electromagnetic fields induced by Helmholtz aiding coils inside saline-filled boundaries. Bioelectromagnetics 1983; 4:357. [0111] [31] Panagopoulos D J, Johansson O, Carlo G L. Evaluation of specific absorption rate as a dosimetric quantity for electromagnetic fields bioeffects. PLoS One 2013; 8: e62663. [0112] [32] Coll A M, Ameen J R, Mead D. Postoperative pain assessment tools in day surgery: literature review. J Adv Nurs 2004; 46:124. [0113] [33 ] Bodian C A, Freedman G, Hossain S, Eisenkraft J B, Beilin Y. The visual analog scale for pain: clinical significance in postoperative patients. Anesthesiology 2001; 95:1356. [0114] [34] Lacy C F, Armstrong L L, Goldman M P, et al. Drug Information Handbook. 15th ed. Hudson, O H: Lexicomp; 2007. [0115] [35] Crocetti S, Beyer C, Schade G, Egli M, Fro\ddot{} hlich J, Franco-Obrego'n A. Low intensity and frequency pulsed electromagnetic fields selectively impair breast cancer cell viability. PLoS One 2013; 8:e72944. [0116 ] [36] Ross C L, Harrison B S. Effect of pulsed electromagnetic field on inflammatory pathway markers in RAW 264.7 murine macrophages. J Inflamm Res 2013; 6:45. [0117] [37] Yurdagul A Jr, Chen J, Funk S D, Albert P, Kevil C G, On A W. Altered nitric oxide production mediates matrix-specific PAK2 and NF-kB activation by flow. Mol Biol Cell 2013; 24:398. [0118] [38] Ha K S, Kim K M, Kwon Y G, et al. Nitric oxide prevents 6-hydroxydopamine-induced apoptosis in PC12 cells through cGMP-dependent PI3 kinase/Akt activation. FASEB J 2003; 17:1036. [0119] [39] Chung H, Dai T, Sharma S K, Huang Y Y, Carroll J D, Hamblin M R. The nuts and bolts of low-level laser (light) therapy. Ann Biomed Eng 2012; 40:516. [0120] [40] Xuan W, Vatansever F, Huang L, et al. Transcranial low-level laser therapy improves neurological performance in traumatic brain injury in mice: effect of treatment repetition regimen. PLoS One 2013; 8:e53454. [0121] [41] Liu S S, Richman J M, Thirlby R C, Wu C L. Efficacy of continuous wound catheters delivering local anesthetic for postoperative analgesia: a quantitative and qualitative systematic review of randomized controlled trials. J Am Coll Surg 2006; 203:914. [0122] [42] Taylor, Hardy, Alonso, Pilla, and Rhode, “Pulsed Electromagnetic Fields Dosing Impacts Postoperative Pain in Breast Reduction Patients”, Journal of Surgical Research (2014) Oct. 9, 2014.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, unless otherwise indicated, a numeric value may have a value that is +/− 0.1% of the stated value (or range of values), +/− -1% of the stated value (or range of values), +/− 2% of the stated value (or range of values), +/− 5% of the stated value (or range of values), +/− 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method for controlling levels of glucose and lipids in the blood of a patient having non-alcoholic steatohepatitis, the method comprising:
generating a pulsed electromagnetic field;
applying the pulsed electromagnetic field in proximity to a target structure in the patient's liver for a predetermined treatment interval.

2. The method of claim 1, wherein the predetermined treatment interval is between 1 and 30 minutes.

3. The method of claim 1, wherein the predetermined treatment interval is between 10 and 20 minutes.

4. The method of claim 1, wherein the predetermined treatment interval is about 15 minutes.

5. The method of claim 1, wherein the treatment interval is repeated 1 to 12 times per day every day for a predetermined number of weeks.

6. The method of claim 1, wherein the treatment interval is repeated 3 times per day for a one to two week period.

7. The method of claim 6, wherein:
a one week treatment period results in the patient having higher levels of triglycerides, HDL cholesterol, and LDL cholesterol in the blood than a similar patient receiving a sham treatment over the same period.

8. The method of claim 6, wherein:
a two week treatment period results in the patient having a heigher level of glucose in the blood than a similar patient receiving a sham treatment over the same period.

9. The method of claim 1, wherein generating a pulsed electromagnetic field comprises emitting a signal comprising bursts of at least one of sinusoidal, rectangular, chaotic, and random waveforms, having a frequency content in a range of about 0.01 Hz to about 100 MHz at about 1 to about 100,000 waveforms per second, having a burst duration from about 1 μsec to about 100 msec, and having a burst repetition rate from about 0.01 to about 1000 bursts/second.

10. The method of claim 9, wherein the signal comprises bursts of a 27.12 MHz sinusoidal wave.

11. The method of claim 9, wherein the burst duration is at least .5 msec.

13. The method of claim 9, wherein the burst duration is about 2 msec.

12. The method of claim 9, wherein the burst repetition rate is between 1 and 20 bursts/sec.

13. The method of claim 9, wherein the burst repetition rate is between 1 and 10 bursts/sec.

14. The method of claim 9, wherein the burst repetition rate is about 2 burst/sec.

15. The method of claim 9, wherein the signal induces a magnetic field having a strength of between 2 and 20 μT in the target structure.

16. The method of claim 9, wherein the signal induces a magnetic field of about 5 μT in the target structure.

17. The method of claim 6, wherein each treatment interval is followed by an inter-treatment interval that is at least 6 times longer than the treatment interval.

18. The method of claim 6 wherein each treatment interval is followed by an inter-treatment interval at least 12 times longer than the treatment interval.

19. A method for controlling levels of glucose and lipids in the blood of a patient having non-alcoholic steatohepatitis, the method comprising:
emitting a pulsed electromagnetic a signal, wherein the signal comprises bursts of at least one of sinusoidal, rectangular, chaotic, and random waveforms, having a frequency content in a range of about 0.01 Hz to about 100 MHz at about 1 to about 100,000 waveforms per second, having a burst duration from about 1 μsec to about 100 msec, and having a burst repetition rate from about 0.01 to about 1000 bursts/second;
applying the pulsed electromagnetic field in proximity to a target structure in the patient's liver for a treatment interval of between 1 and 30 minutes; and
repeating the treatment interval between 1 and 12 times per day every day for a predetermined number of weeks, wherein each treatment interval is followed by an inter-treatment interval that is at least 6 times longer than the treatment interval

20. The method of claim 19, wherein:
the signal comprises bursts of a a 27.12 MHz sinusoidal wave, each burst having a duration of about 2 msec and a burst duration rate of about 2 bursts/sec;
the signal induces a magnetic field of about 5 μT in the target structure;
the treatment interval is about 15 minutes, repeated 3 times a day for one to two weeks; and
the inter-treatment interval is about three hours.
